(19) United States
(12) Patent Application Publication
(10) Pub. No.: US 2019/0122760 A1
(43) Pub. Date: Apr. 25, 2019

(54) METHOD AND SYSTEM FOR CUSTOMIZED SCHEDULING OF HOME HEALTH CARE SERVICES

(71) Applicant: Kevin Sunlin Wang, Flushing, NY (US)

(72) Inventor: Kevin Sunlin Wang, Flushing, NY (US)

(21) Appl. No.: 16/219,927

(22) Filed: Dec. 13, 2018

Related U.S. Application Data
(63) Continuation-in-part of application No. 15/715,012, filed on Sep. 25, 2017.
Application No. 15/715,012 is a continuation-in-part of application No. 15/239,783, filed on Aug. 17, 2016.
(60) Provisional application No. 62/399,129, filed on Sep. 23, 2016.
(60) Provisional application No. 62/505,626, filed on May 12, 2017.
(60) Provisional application No. 62/325,602, filed on Apr. 21, 2016.
(60) Provisional application No. 62/290,778, filed on Feb. 3, 2016.

(51) Int. Cl. G16H 40/20; G16H 10/60; G06Q 40/08

(52) U.S. Cl. CPC G16H 40/20; G06Q 10/1093; G06Q 40/08; G16H 10/60

(57) ABSTRACT

A method and system provides automated and customized prescheduling home health care services based on caregiver availability and caregiver compatibility with individual patients without or with less live coordinator involvement. A caregiver and patient may be matched with one another based on priority rankings of favorite lists and/or preferred lists, and/or based on preferences or limitations. Patients and caregivers may also anonymously create blacklists to exclude one another from being matched together without the excluded party finding out that he or she has been blacklisted. Service requests are received in advance of a designated scheduling date, prescheduled, and/or batched based on caregiver availability and customizable parameters and/or preferences to establish priority and/or compatibility between individual patients and caregivers.

### CROSS-REFERENCE TO RELATED APPLICATIONS

The present application is a continuation-in-part of U.S. Non-Provisional patent application Ser. No. 15/715,012, filed on Sep. 25, 2017 and titled System and Method For Customizable Prescheduled Dispatching For Transportation Services, which claims priority to U.S. Provisional Patent Application No. 62/399,129, filed Sep. 23, 2016, and U.S. Provisional Patent Application No. 62/505,626, filed May 12, 2017, and is a continuation-in-part of U.S. Non-Provisional patent application Ser. No. 15/239,783 (hereafter, the '783 application) filed on Aug. 17, 2016, which claims the benefit of U.S. Provisional Patent Application No. 62/325,602, filed on Apr. 21, 2016 and U.S. Provisional Patent Application No. 62/290,778, filed Feb. 3, 2016, the disclosures of which are all hereby incorporated by reference herein in their entireties.

### FIELD OF THE INVENTION

The present disclosure generally relates to systems and methods for dispatching/scheduling home health care services, and more particularly, to systems and methods for on-demand and prescheduled home health care services in which one or more service requests are received, scheduled on-demand, prescheduled, and/or grouped based on caregiver availability and customizable parameters and/or preferences to establish priority and/or compatibility between individual patients and caregivers.

### BACKGROUND

Scheduling software in the home health care services industry is programmed to deal with different variables relating to caregivers and patients, including processing service requests received from both patients and vendors (e.g., insurance companies) and storing information associated with the service requests, the caregivers, and the patients to assist health care coordinators to assign caregivers to particular patients. However, this software is extremely inefficient in terms of the types of information it collects and stores for later processing by coordinators when creating schedules. Errors or conflicts can slow processing speeds and compound problems that may eventually obstruct service. Organizing such information in a database and comprehensively querying it to preschedule service requests in an efficient manner presents numerous challenges, particularly where coordinators sort through one caregiver after another until a preliminary match is found. The caregiver must then be contacted to see if he/she is actually able to provide the service. Alternatively, if the caregiver receives a list of already-scheduled services, he/she may have to cancel one or more if a conflict arises. Additionally, some caregivers may work for multiple agencies and attempt to collect payment from the multiple agencies for services allegedly performed on the same day at the same time, either individually or in collusion with one another.

In conventional systems, such problems can be extremely confusing and time consuming for a coordinator to preschedule services the day before, or taxing on a computing system. The process is further complicated when cancellations and booking changes are factored in, especially when there are multiple duties per service (e.g., cooking, cleaning, showering, taking a patient from his/her home to an appointment and then back home, etc.), simultaneous service for more than one patient, or multiple services scheduled back to back. Changes or cancellations may necessitate that a service request be sent back for re-scheduling, often manually.

When presented with a service request, a caregiver may assume that he/she will be able to complete it on time, but due to an unforeseen change or miscalculation, he/she may have to work past his/her desired stopping time. Thus, caregivers are often scheduled to service requests for which their schedules are incompatible, and patients and caregivers sometimes have to cancel with little advanced notice. Coordinators are not always familiar with who the patients are, and may not know their preferences. Situations arise where a coordinator sends the caregiver living the closest to the patient to the patient, but the patient may not like that particular caregiver. Although the caregiver is in close proximity and can quickly travel to the patient on short notice, the patient may be dissatisfied with the service. If the patient complains about the caregiver, provides a low rating, requests a new caregiver, or requests termination of a particular caregiver, such actions may result in confrontation, conflict, or at a minimum, awkwardness, between the patient and the caregiver depending on the nature of service, the amount of money involved, and the relationship between them. Additionally, the patient may be significantly older than the caregiver, and have no desire to upset the caregiver or risk any sort of confrontation for giving negative reviews, ratings, etc. Low ratings or reviews may create additional problems for caregivers as this may affect their overall ratings or reviews, resulting in loss of business from prospective patients. As coordinators may know the caregivers personally, patients may feel a lack of control over who is administering care to them, or forced to select someone who does not really feel like a match.

Caregivers who are prescheduled for a service often have to wait extended periods of time for a patient who is not yet ready, even though the caregiver was scheduled to show up at a certain time. Coordinators have no way of knowing or efficiently updating the caregiver as to when the patient may be ready. Dispatching or scheduling home health care services is a very complicated operation and has numerous areas in need of systematic improvement. Traditional scheduling methods allow for little adaptability to new service requests and/or service requests that change unexpectedly.

It will therefore be appreciated that improved systems and methods are needed in the art to address these issues, streamline processes, increase caregiver utilization, improve efficiency, limit manual dispatching/scheduling, address patient and caregiver dissatisfaction, enable patient and caregiver customization for on-demand and prescheduled service request assignments, and utilize technological improvements for on-demand and prescheduled services with automation.

### SUMMARY OF THE INVENTION

This summary is not intended to identify or point to essential features or limit the scope of the subject matter claimed herein. The present invention relates to various systems and methodologies of a prescheduling and on-demand computer-implemented system for scheduling home health care services in which a single service request or a plurality of service requests are received on the day of or in advance of a designated dispatch/service date, scheduled on-demand, prescheduled, and/or possibly grouped based on caregiver availability and customizable parameters and/or preferences to establish priority and/or compatibility between individual patients and caregivers in a manner which solves various deficiencies in known home healthcare scheduling and dispatch service systems, with at least the following objectives:

To enable full home healthcare service scheduling, which includes scheduling service requests on-demand, prescheduling service requests, dispatching service requests, dynamically updating and facilitating changes to service requests, and providing real time information to all parties during service thereof;

To provide a customizable prescheduled home health care service method and system, whereby a caregiver and a patient are most appropriately matched based on a predetermined set of rules that determine the dispatch system's output based on known patient and caregiver preferences and/or limitations;

To provide a customizable scheduling method and system having established a set of predetermined rules that determine matches and assignments of caregivers and patients;

To provide a system configured to dynamically update a database to indicate changes in patient and caregiver presets, settings, preferences, limitations, feedback or other information in order to provide quality services;

To enable patients to customize options within the system based on the types of preferences they have in order to receive the best service from one or more best matching caregivers;

To facilitate the development and nurturing of a relationship between a patient and a caregiver by creating a favorites list and a preferred list based on their selections, which may be preset in the system and/or updated following completion of service requests such that favorite caregivers may be given priority to accept service requests from their favorite patients who are on their favorites list;

To enable patients to customize options within a system based on the day of week or the time of day in order to receive the best service from the best matching caregiver;

To enable customized scheduling of healthcare providers based on a variety of patient needs and/or duties required, such as cooking, cleaning, showering, transportation (e.g., taking a patient from home to a store and then back home), simultaneous service for more than one patient, multiple services scheduled back-to-back, etc., including a caregiver's physical and mental attributes (e.g., size, strength, capabilities, level of experience, etc.);

To enable caregivers to prioritize/rank the list of patients on their favorite patient lists or patients to prioritize/rank the list of caregivers on their favorite caregiver lists, in order to resolve any conflicts arising during the prescheduling process and facilitate automated matching and scheduling;

To enable patients or caregivers to prioritize their categories of preferences or limitations, respectively, in order to further resolve any conflicts arising during the prescheduling process and facilitate automated matching and scheduling;

To create blacklists or block lists whereby caregivers or patients will be skipped over during various matching methodologies;

To provide various sets of indicators to a patient and/or a caregiver to facilitate selecting a best match for a single service request in a convenient and efficient way;

To enable a price negotiation for a single service request between a patient and one or more best matching caregivers;

To facilitate a micro-scheduling system between caregivers and replacement caregivers to substantially reduce the workload of a traditional dispatcher, coordinator, and/or coordination system;

To enable automated prescheduling of a batch of service requests to one or more caregivers who have each preset one or more geographic regions where they prefer to provide service;

To provide a special purpose device that may be used for tracking home health care patients, and caregivers to enable verification of billing information associated with caregivers providing service to patients;

To facilitate connectivity between a coordinator and caregivers by displaying changes to a prescheduled service request in a scheduling web portal for the coordinator, and in a caregiver device associated with a caregiver assigned to the service request, and dynamically updating and storing the changes in a database without any phone call communication;

To enable caregivers to prioritize or rank the list of patients on their favorite patient list, and enable patients to prioritize or rank the list of caregivers on their favorite caregiver list, in order to resolve any conflicts arising during the scheduling process; and

To enable caregivers to prioritize or rank the list of patients on their preferred patient list, and enable patients to prioritize or rank the list of caregivers on their preferred caregiver list, in order to resolve any conflicts arising during the prescheduling process.

In accordance with one aspect of the invention, a computer-implemented method provides customizable automated prescheduling of home health care services. The method comprises receiving, by a server, a plurality of preset caregiver service zones, wherein each preset caregiver service zone is defined by one or more geographic regions; receiving, by the server, a plurality of preset caregiver preferences or limitations of a plurality of caregivers; receiving, by the server, a plurality of optionally preset patient preferences of a plurality of patients; storing, in a database, the plurality of preset caregiver service zones, the plurality of preset caregiver preferences or limitations, and the plurality of optionally preset patient preferences; receiving, by the server, a plurality of service requests for prescheduling, wherein each service request corresponds to one of the plurality of patients and contains time-location data associated with at least one service location; and parsing, by the server, the plurality of service requests into one or more batches, wherein each batch corresponds to a respective preset caregiver service zone and includes one or more of the plurality of service requests having service locations all within the respective preset caregiver service zone. The method further comprises prescheduling the plurality of service requests by, for each of the one or more batches: (i) retrieving, from the database, a set of the plurality of caregivers associated with the respective preset caregiver service zone corresponding to the batch; (ii) automatically establishing, by the server, in accordance with the one or more predetermined rules, for each respective service request of the batch, a weighted priority for each caregiver of the set, wherein the weighted priority is based on assigned weights for at least one service relevant factor associated with the respective service request, and wherein the at least one service relevant factor includes at least one of the plurality of preset caregiver preferences or limitations or at least one of the optionally preset patient preferences of the patient corresponding to the service request; and (iii) applying, by the server, the one or more predetermined rules to assign a caregiver from the set to the respective service request based on the weighted priority of the caregiver; transmitting, by the server to a computing device of a particular caregiver, a dispatch notification which includes one or more assigned service requests; and receiving, by the server from the computing device of the particular caregiver, an acceptance of at least one of the assigned service requests.

In accordance with another aspect of the invention, a computer-implemented method provides customizable automated prescheduling for home health care services. The method comprises receiving, from a patient device associated with a patient: a first input requesting that a particular caregiver be placed on a blacklist of the patient, and a second input corresponding to at least one patient preset preference comprising: a favorite caregiver, a preferred caregiver, or a customizable caregiver attribute; adding the particular caregiver to the blacklist of the patient; receiving, from a plurality of caregiver computing devices associated with a plurality of caregivers: a plurality of caregiver preset preferences corresponding to the plurality of caregivers, wherein the plurality of caregiver preset preferences comprise at least one of: a favorite patient, a preferred patient, or a geographic region where a respective caregiver desires to provide services to patients; receiving at least one preset calendar preference including one or more designated time periods during which the plurality of caregiver preset preferences are applicable, or one or more designated time periods during which the at least one patient preset preference is applicable; storing the first input, the second input, the plurality of caregiver preset preferences, the blacklist, and the at least one preset calendar preference in a database; receiving a service request associated with the patient for scheduling, the service request including at least one of: number of hours, authorization level, certification level, type of care, service start time, service location, or service end time; and automatically scheduling a caregiver to service the service request based on the at least one patient preset preference, the at least one caregiver preset preference, and the at least one calendar preference, wherein the particular caregiver is precluded from being scheduled to the service request based on the first input, and wherein the blacklist of the patient is accessible by the patient.

In accordance with yet another aspect of the invention, a computer-implemented method provides customizable automated prescheduling for home health care services. The method comprises receiving, from a caregiver device associated with a particular caregiver, a first input requesting that a particular patient be placed on a blacklist of the particular caregiver; receiving, from a patient device of the particular patient, a second input corresponding to at least one patient preset preference comprising: a favorite caregiver, a preferred caregiver, or a customizable caregiver attribute; adding the particular patient to the blacklist of the particular caregiver; receiving, from a plurality of caregiver computing devices associated with a plurality of caregivers: a plurality of caregiver preset preferences corresponding to the plurality of caregivers, wherein the plurality of caregiver preset preferences comprise at least one of: a favorite patient, a preferred patient, or a geographic region where a respective caregiver desires to provide services to patients; receiving at least one preset calendar preference including one or more designated time periods during which the plurality of caregiver preset preferences are applicable, or one or more designated time periods during which the at least one patient preset preference is applicable; storing the first input, the second input, the plurality of caregiver preset preferences, the blacklist, and the at least one preset calendar preference in a database; receiving a service request associated with the particular patient for scheduling, the service request including at least one of: number of hours, authorization level, certification level, type of care, service start time, service location, or service end time; and automatically scheduling a caregiver to service the service request based on the at least one patient preset preference, the at least one caregiver preset preference, and the at least one calendar preference, wherein the particular caregiver is precluded from being scheduled to the service request based on the first input, and wherein blacklisting of the particular patient by the particular caregiver is accessible by the particular caregiver.

Other objects, features, and characteristics of the present invention, as well as the methods of operation and functions of the related structural elements, and the combination of parts and economies of development and manufacture, will become more apparent upon consideration of the detailed description below with reference to the accompanying drawings, all of which form a part of this specification.

### BRIEF DESCRIPTION OF THE DRAWINGS

A further understanding of the present invention can be obtained by reference to a preferred embodiment set forth in the illustrations of the accompanying drawings. The drawings are not intended to limit the scope of this invention, which is set forth with particularity in the claims as appended or as subsequently amended, but merely to clarify and exemplify the invention. Accordingly, a more complete appreciation of the present invention and many of the attendant aspects thereof may be readily obtained as the same becomes better understood by reference to the following detailed description when considered with the accompanying drawings, where:

FIG. 1 is a diagram of an exemplary prescheduled dispatch system, including a computing system and one or more peripheral computing devices for use with various embodiments of the invention;

FIG. 2 is a diagram of an exemplary computing device and associated components in accordance with various embodiments of the invention;

FIG. 3 is a diagram depicting a dispatch operation incorporating an exemplary dispatch matrix in accordance with various embodiments of the invention;

FIG. 4A is a flowchart illustrating exemplary dispatch logic in accordance with various embodiments of the invention;

FIG. 4B is a flowchart illustrating an exemplary methodology incorporating patient or caregiver cancellation in accordance with various embodiments of the invention;

FIG. 5 is a flowchart illustrating an exemplary price negotiation between a caregiver and a patient with or without a coordinator web portal;

FIG. 6 is a flowchart illustrating an exemplary methodology for assigning a plurality of prescheduled service requests in accordance with various embodiments of the invention;

FIGS. 7A and 7B are flowcharts illustrating an exemplary methodology for assigning a plurality of prescheduled service requests based on a first algorithm in accordance with various embodiments of the invention;

FIG. 8 is a flowchart illustrating an exemplary methodology for assigning a plurality of prescheduled service requests based on a second algorithm in accordance with various embodiments of the invention;

FIG. 9 is a flowchart illustrating an exemplary methodology for prescheduling and rescheduling a plurality of service requests using replacement caregiver and on-demand methodologies in accordance with various embodiments of the invention;

FIG. 10 illustrates an exemplary electronic map interface showing unassigned service requests in different geographic regions in accordance with various embodiments of the invention;

FIG. 11A illustrates an exemplary electronic user interface for allowing a patient to add one or more caregivers to the patient's blacklist; and

FIG. 11B illustrates an exemplary electronic user interface for allowing a caregiver to add one or more patients to the caregiver's blacklist.

### DETAILED DESCRIPTION OF THE PREFERRED EMBODIMENT

The present disclosure is not intended to be limited to the specific terminology so selected, and it is to be understood that each specific element includes all technical equivalents which operate in a similar manner. Specific embodiments that may be practiced are shown by way of illustration and explanation. The embodiments are described in sufficient detail to enable those skilled in the art to practice the embodiments, and it is to be understood that the logical, mechanical, and other changes may be made without departing from the scope of the embodiments. The following detailed description is therefore not to be taken in a limiting sense. In describing exemplary embodiments of the present invention illustrated in the drawings, specific terminology is employed for sake of clarity.

Systems and methods for providing dispatch home healthcare services are described herein. It will be appreciated by one of ordinary skill in the art that one or more of such exemplary embodiments may be carried out using various programmatic engines, programmatic modules, or other hardware or software components comprising one or more routines or one or more subroutines, which may be used individually or in combination and carried out on any appropriate technological means.

It will also be appreciated that various modules of the systems and methods described herein may be implemented in part by using an interfacing mobile application (app) on an internet-enabled mobile device's operating system, such as, for example, Android, iOS, or Windows Phone OS, and in part by using a web portal interface, and that different types of users may utilize different functionalities of the system.

“Users” or “subscribers” as recited herein can include, for example, one or more caregivers or patients, third parties, third party entities, or other service providers acting on behalf of or for the caregiver or patient, or one or more individuals from such third-party entity or service provider who requests or orders services on behalf of a patient.

“Patients” as recited herein can include anyone, including, for example, one or more individuals, such as anyone who registers with the system in need of a home healthcare service, regardless of which type of services may be needed. As patients and caregivers alike can use the methods and systems described herein, both may generally be referred to as a user or users, and may additionally be referred to as having a specific user type corresponding to the role they play in the service request.

“Services” as recited herein can refer to three types of services: (i) at-home services for patients, which can comprise any number of duties, such as, but not limited to, cooking, cleaning, laundry, helping patients shower and dress, nursing work, counseling, physical therapy, or any other at-home service the patient needs, (ii) delivery service (e.g., grocery shopping), and (iii) transport service (e.g., if the patient desires to go grocery shopping but cannot do so on his/her own, or if the patient needs to be brought to an appointment, etc.). Such services can be prescheduled or requested on-demand.

“On-demand” and “prescheduled” as recited herein are meant to further define when services take place (i.e., in real-time, as requested, or prescheduled). On-demand is used in opposition to the notion of being prescheduled or occurring at a future fixed time, such as a couple hours or days later. On-demand services happen at the moment in real-time, whereas prescheduled services take place at a predetermined future time, date, or during a predetermined timeframe.

“Customized” as recited herein refers to modification of the nature of services provided, and refers to the preferences of a patient and/or the preferences or limitations of a caregiver involved in the services, on an individual basis. Regardless of the type of service, a “service provider” herein may be a single individual such as a caregiver, a group of people, or an affiliate of a private business entity such as a home healthcare service company who can be summoned to provide home health care services.

“Coordinator(s)” as used herein can include an individual or group of individuals who manage service request(s) and operate various scheduling and prescheduling functionalities. Vendors herein can refer to a broker or other business entity, government office, or individual who brokers a service on behalf of a patient. As described herein, each of these different roles may be referred to specifically or by the catch-all terms user or users that register in or are otherwise directly or indirectly associated with the system.

A “request” as recited herein for any type of service is generally referred to as a “service request” or “service(s)” throughout the disclosure. A service request is assigned to a caregiver based on a patient's and/or caregiver's preferences and/or limitations, and priority is given to certain caregivers for certain services with patients based on one or more attributes or prior services.

“Preference” as used herein refers to various factors about a particular service that a patient would like included in the overall service provided to him/her in terms of the particular caregiver, the particular vehicle, or form of transportation (i.e., public buses, trains, taxis, etc.) the caregiver uses, and any attributes related thereto. These preferences may represent ideal or favored conditions for the service request. Alternatively, such preferences may represent preconditions for service. A patient can set his/her preferences on a mobile device and update them at any point, allowing the patient to feel comfortable, and in the end, satisfied with the quality of the services received. A patient can have one or more such preferences.

In home healthcare agencies, employees provide services to patients who live at home. The primary employee groups may include Administrator, Coordinators, Human Resources (HR) (which can include intake and compliance officers), Caregivers, Billing (which can include pre-billing), and Training schools. One or two Administrators are generally used per agency and can be provided full access to any systems being utilized. An Administrator can work on-site at the agency, and is usually the person who reviews errors and in-application notifications or issues, creates employee groups, and manages audits. The Administrator may preset how much information can be shown to other groups of employees in accordance with HIPAA (Health Insurance Portability and Accountability Act), which governs how PHI (Protected Health Information) can be disclosed. For example, any information identifying a patient should only be given to the Caregiver and Coordinator involved in the care of that patient. Additionally, a Caregiver must be careful not to share patient identifying information with anyone. In large agencies, the Administrator may set up teams of Coordinators, usually alphanumerically. Each Coordinator team may have a team leader and a few Coordinators working under the team leader responsible for dispatching Caregivers in accordance with their preferences and location. Coordinators should not see the PHI of another Coordinator's patients despite working in the same team. The Administrator is also responsible for setting permission levels in terms of which PHI billing personnel can view the information. For example, billing personnel may need to see a patients' schedules in order to properly bill batches of services provided to the insurance company, but do not need to know all medical information involved with some patients.

Coordinators usually work in teams to dispatch all caregivers to their preferred patients or locations if possible. Coordinators review authorizations and how much service is to be provided for each patient, and look for Caregivers whose schedules fit into varying time frames. Coordinators often extensively use calendar functionality and employee verification to ensure caregivers are working the times that they were assigned, and not participating in fraudulent activities. If a Caregiver's time cannot be tracked, then that Caregiver might not be properly paid. Caregivers are the agency employees who visit patients' homes to provide care. Different caregivers have different areas of expertise, and levels of proficiency in each area varies. Generally, the higher the proficiency of a Caregiver in a particular field, the higher the pay. As Caregivers sometimes work for multiple agencies in a day or a week, tracking their working hours is imperative. Caregivers may have more than one patient in a day, and report back to Coordinators for any planning or wage discrepancies.

Billing personnel are responsible for batching and sending service claims to the insurance company for payment, a portion of which include Caregiver wages. Additionally, some agencies further split Billing personnel into Pre-Billing personnel, who look for any errors in the batches of service claims generated, go over the physical timesheets that the Caregivers hand in, and match them to times verified on an electronic calendar. Any issues are typically sent back to the Coordinators to resolve first. Patients are the individuals who receive care based on an authorization from a doctor or an insurance company. Patients generally stay at home and receive the Caregiver, who arrives at the Patient's home to provide services. Such services may include, for example, housework, resident nurse work (RN), laundry, cleaning, showering, cooking, etc. The type of Caregiver dispatched typically depends on the type(s) and amount of service(s) each patient requires. For example, if the patient requires physical assistance, the particular caregiver's size, strength, or other physical attributes may be considered when assigning the appropriate caregiver. The HR department handles internal conflicts within the agency, and employs a Compliance Officer to review the work of other employees to avoid fraud and mistakes. Various Training Schools for Caregivers may be provided on-site at the location of the agency or at a local area. The agency may employ people to provide lessons for the Caregivers to receive their certifications.

It will be appreciated that tracking and dynamically updating patient and caregiver profiles, preferences and limitations using the systems disclosed herein, and dispatching caregivers in accordance with the methods disclosed herein, can significantly reduce the dispatch, organizational, and legal demands of both Coordinators and Administrators, as well as the demands and computation required of a computer system when scheduling or prescheduling service request. By pre-loading such preferences and limitations and dynamically updating them in a computing system, the computing system can run algorithms (further described herein) based on readily accessible information without having to separately search for, analyze, and compute such underlying information for each service request. Caregivers can be matched to patients, and both Caregivers and Patients can preset their most favorite and least desired caregivers through respective favorite lists and blacklists. The parameters of service requests of Patients may include a number of hours or units of time needed, an authorization level, a certification level, one or more work items, a type of care, a visit start time, a visit location, and/or a visit end time. Caregivers and Patients may similarly be assigned to one another's favorite lists, preferred lists, or blacklists as described herein. In this manner, the system can determine whether, on a particular designated date, optionally preset patient preferences match optionally preset caregiver preferences, and matching can be used in accordance with customized algorithms as described herein to preschedule a caregiver for a patient or find a best match on-demand.

“Limitations” as used herein can mean any type of constraint that a caregiver may wish to place upon a service he/she provides. These limitations may include two broad categories: personal limitations regarding when, where, how, and to whom a caregiver provides services. Such personal limitations may include location limitations based on a caregiver's reluctance to provide services in certain geographic zones, and time limitations based on a caregiver's reluctance to provide one or more services within certain time periods. Alternatively, in certain preferred embodiments, a caregiver may also specify one or more preset location preferences based on a caregiver's desire to provide services to (e.g., preference to receive work within) certain geographic zones. In such embodiments, the caregiver may also specify location limitations within his/her location preferences based on the caregiver's reluctance or inability to provide services in certain geographic zones within the caregiver's preferred geographic zone of service. Service limitations may additionally or alternatively relate to times, time frames, days of the week or days of the month when the caregiver might not want to provide service to a patient. Such times can include hours of the day, days of the week, times of year, etc. Caregivers can also set limitations for location and time and day of week or day of month or day of year. By way of example, a caregiver may not want to provide service in a certain area past 10 PM, but may be willing to provide service in another area past that time. The caregiver may also specify time preferences indicative of a time period during which the caregiver prefers to receive service requests. The caregiver may also specify whether they want to prioritize servicing more patients per day or prioritize certain patients based on the price of a certain service request. A caregiver can have one or more such limitations and/or preferences, and the terms are not intended to limit a patient or caregiver to only having a preference or a limitation, but rather they are intended to draw an illustrative contrast.

Multiple terms are used herein to refer to a patient who has established a positive relationship with a caregiver (e.g., as desired by the caregiver) and who has also placed the caregiver on his/her favorite caregiver list. A favorite patient is one who is on a caregiver's favorite patient list. A caregiver who has established a positive relationship with a patient is referred to as a favorite caregiver (e.g., on a patient's favorite list).

“Favorite” as used herein is meant to be broadly construed, and refers to any patient or caregiver who is prioritized in an assignment of a service request (e.g., on a favorite list), or assigned a particular one of different types of services requested in a service request. It will be understood by one skilled in the art that the concept of a “favorite list” may alternatively be referenced as, for example, a friend, a top, a priority, or any other word used by caregivers or patients to define this concept of ranking patients and caregivers. Regardless of specific terminology, the intention is to facilitate a positive caregiver-patient relationship which encompasses matching between caregivers and patients. Preferably, to be classified as a favorite caregiver or patient, each of the patient and caregiver must list one another on their respective favorites' lists. If only one of either the patient or the caregiver lists the other on his/her favorites list, then the relationship becomes preferred. If neither list the other on their favorite's list, then the relationship becomes a regular relationship. Preferably, to be classified as a “favorite caregiver” or “favorite patient”, each of the patient and the caregiver must list the other on his/her respective favorites list. If only one of either the patient or the caregiver lists the other on his/her favorites list, then the relationship becomes preferred. If neither lists the other on his/her favorites list, then the relationship becomes regular relationship.

In contrast to favorite, a “caregiver's blacklist” or a “patient's blacklist” as used herein refers to lists which can prevent a match between a caregiver and a patient in the future (e.g., where both are precluded from being matched with one another during service request processing). It will be appreciated by one skilled in the art that other terms may be used to describe this concept, such as, for example, block list, ban list, dislike list, or the like. Regardless of specific terminology, the intention is to enable a caregiver to exclude patients to whom he/she does not want to provide service, or with whom he/she does not want to have contact, and a way for a patient to exclude the caregivers from whom he/she does not want service or contact.

A preferred caregiver may also be one whom the patient has directly requested be on his/her preferred list, or one whom the patient has requested be on his/her favorite's list, but whose request has not been agreed to by the caregiver. In either case, if the caregiver refuses a patient's request, then the caregiver will not be added to the patient's favorite or preferred list, and vice versa. The word preferred as used herein generally refers to a lower ranking than favorite for purposes of matching patients and caregivers when assigning prescheduled service requests. When assigning jobs to caregivers, a favorite relationship preferably takes precedent over a preferred relationship. Should the relationships be equal between a patient and two caregivers, the system may be configured to look to historical or other data to determine which caregiver has more services with the particular patient, or may automatically assign a caregiver in accordance with a customizable algorithm and preset preferences (further discussed below). For example, the caregiver having had more satisfactory services with a particular patient may be the preferred caregiver for the particular job. Alternatively, the system may be configured to review the specific service request and identify a caregiver who has the most experience or familiarity with the patient and his/her needs, requests, personality, etc. In certain scenarios, the caregiver having the greater familiarity with the specific patient may be given priority over a caregiver having more experience with a particular issue generally. Alternatively, the patient may be provided with the option to choose which of the two or more caregiver's he/she prefers for the specific service request. Caregivers and patients may further rank each other within these classifications.

Drop-off locations can be complicated, such as, for example, at hospitals or clinics which often have confusing or unclear layouts. At these locations, it can be difficult to locate the correct entrance. Thus, it may be preferred to dispatch a service request to a caregiver who is familiar with a particular destination location or route when this type of location is involved. Any caregiver who is not a favorite caregiver or a preferred caregiver of a patient is referred to herein as a “regular caregiver”. This term essentially means that the caregiver is not a preferred caregiver and has not been added to the favorite list. It will be understood that a particular status relating to a caregiver (i.e., caregiver type), be it favorite, preferred or regular, is always also relative. A caregiver who is preferred for one patient might be a favorite for another.

“System” as used herein is referred to the implementation through a combination of hardware and software that operates a portable computing device, which comprises various pre-programmed features combined and integrated with basic components, including but not limited to one or more servers, databases, mobile end applications, web portals, network settings, etc. With the support of these components, the system provides the services through user interfaces, such as a website or a mobile application. Various embodiments of systems described herein provide prescheduled or on-demand dispatching through a computing system. The computing systems described herein can include any combination of hardware and software, and can communicate with a plurality of portable computing devices through, for example, a wide-area, packet switched network, which can allow users to access various pre-programmed features combined and integrated with basic components. Such features and components can include but are not limited to one or more servers, databases, mobile end applications, web portals, network settings, etc. within a communications framework/network. With the support of these components, the systems described herein provide services through user interfaces, such as, for example, a website or a mobile application on a portable computing device. The systems may also include more than one server operatively disposed in a distributed structure with support from data centers that may be located anywhere around the world.

Certain embodiments of the systems described herein are not limited to applications involving conventional computer programs or programmable apparatuses that run them. It is contemplated, for example, that embodiments of the inventive disclosure could include an optical computer, a quantum computer, an analog computer, or the like.

Each element in the flowcharts herein depicts a step or a group of steps of a computer-implemented method, and each step may contain one or more sub-steps. For purposes of illustration, these steps (as well as any and all other steps identified and described) are presented in a certain logical order. However, it will be appreciated that any exemplary embodiment described herein can contain an alternate order of the steps adapted to a particular application of a technique disclosed herein, and that any variations and/or modifications are intended to fall within the scope of this inventive disclosure. The depiction and description of steps in any particular order is not intended to exclude embodiments having the steps in a different order, unless required by a particular application, explicitly stated, or otherwise clear from the context.

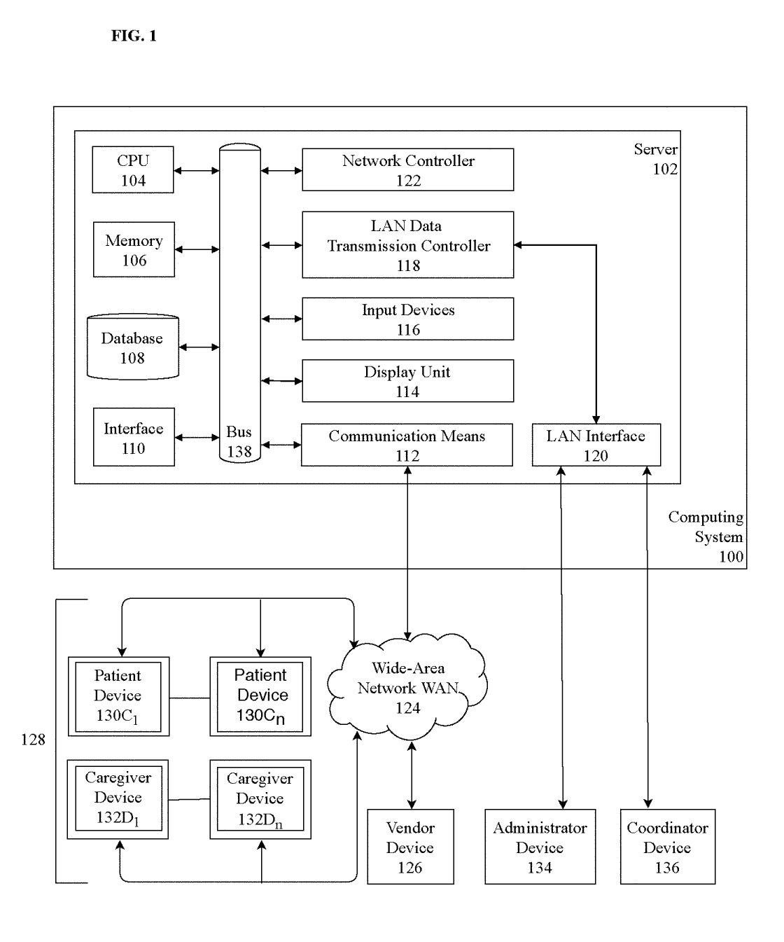

The systems and methods described herein are best understood with reference to the following drawings, which are described in detail below. Referring first to FIG. 1, illustrated is a diagram of an exemplary computing system 100 and a plurality of peripheral computing devices. A combination of hardware and software operates on a plurality of computing devices 128 and the computing system 100, generally with one or more connections to wired or wireless wide area network (WAN) 124 (e.g., the Internet), incorporated with local devices through a local area network (LAN) interface 120. Computing devices 128 can be a wireless mobile hardware device having software capable of communicating information to other mobile devices or computer systems, determining the location of that device with geographical position location capability (e.g., through triangulation of a cell system, GPS, specifically input by a user, etc.), and connecting to a private computer network or public network such as the Internet through a network.

Computing system 100 can include, for example, server 102 including central processing unit (CPU) 104, memory unit 106, database 108, interface 110, communication means 112, display unit 114, one or more input devices 116 (e.g., keyboard, mouse, etc.), LAN data transmission controller 118, LAN interface 120, network controller 122, and internal bus 138. As shown, the system may be connected to a data storage device, such as, for example, a hard disk disposing one or more databases 108 via a wired or wireless link. Central computing system 100 can include one or more servers configured the same or similar to server 102 shown in FIG. 1, or one or more servers configured in a different manner, which may dispose different hardware or software. For example, computing system 100 may comprise multiple servers hosted in multiple spaces such as data centers or server farms.

Computing system 100 may be configured to communicate with network service(s) coordinated through communication means 112, which may include any approach for communicating data over one or more networks or to one or more peripheral devices.

Communication means 112 may include, but is not limited to, circuitry and control systems for providing wireless connections, wired connections, cellular connections, data port connections, Bluetooth connections, or any combination thereof, and the means may include devices enabled to communicate using such communication approaches. One of ordinary skill in the art will appreciate that there are numerous approaches to communications that may be utilized.

Server 102 and computing system 100 may be communicatively linked, through communication means 112 and WAN 124, to peripheral devices such as computing devices 128, vendor device 126, administrator device 134, and coordinator device 136. Computing devices 128 may be configured as one or more patient computing devices 130C1-130Cn or caregiver computing devices 132D1-132Dn. Computing devices 128 may be devices (e.g., smartphone, smartwatch, etc.) which allow a user (e.g., patient, caregiver, etc.) to interact with the computing system 100. Any number (e.g., 1, 2, 3, . . . n) of caregiver devices 132D1 . . . 132Dn, or patient devices 130C1 . . . 130Cn may be used in conjunction with computing system 100.

Alternatively, the systems and methods for home healthcare services as disclosed herein may further include enabling a third-party agent or agency (e.g., an insurance company, etc.) to schedule home healthcare services on behalf of one or more patients directly with caregivers (i.e., independent caregivers, caregivers associated with a dispatch service, etc.) or indirectly with the caregivers through the dispatch service or system. In such an embodiment, a third-party agent may amass a plurality of service requests for a plurality of patients, and with permission of one or more dispatch companies, directly interact or otherwise communicate with such dispatch companies' caregivers to schedule caregivers to the service requests, either on-demand or prescheduled. The third-party agent may interact with a plurality of dispatching companies to increase the number of available caregivers to handle the amassed service requests. A plurality of patients may each initiate a service request with a third-party agent for assignment (i.e., either on-demand or prescheduled) to a plurality of caregivers for completion. Once the service requests are received, the third-party agent, using a computing system, may communicate directly with a plurality of caregivers to determine the best matching caregiver for the respective service requests based on predetermined preferences and/or limitations of the patient(s) and/or caregiver(s), including any preset calendar preferences (e.g., presets for particular days of the week, month or year, presets for days not working because of weekends, holidays, vacations, etc.).

It will also be appreciated that remote computing devices 128 may be non-smartphone (i.e., traditional cellphones, landline telephones, etc.) that may connect with computing system 100 through a network, which may be telephone communication network without the Internet. In such an embodiment, a patient's voice request for home healthcare service is transmitted to the central server where voice recognition is performed and the healthcare request is processed. The system then responds automatically with the caregiver details and estimated time of arrival information. The central server may preferably work in conjunction with a database to store previous service information, such that it may later utilize the data to match the patient's voice to a log of previous service requests by the patient. An automated response by an IVR system may inquire as to whether it is the same service request type that was most previously completed. The patient may be given a chance to choose “yes” or “no” or to select from a number of options, so that the request can be processed automatically. Alternatively, the telephone system may prompt the patient to verbally provide new service request details. Once this is processed, the system will automatically respond with the caregiver details, ETA information, etc.

The method and system may further provide a multi-level IVR system where callers are asked to choose from a series of audio prompts (e.g., “Press 1 for . . . ”), and then based on the caller's selection, he/she may be given a series of audio prompts to choose from, and so on. The caller may continue to be routed through the system until the necessary information is received and/or provided. Multi-level IVRs are customizable, and can handle many prompts and levels of the IVR the callers must go through until their inquiry or request is properly handled by the system or they are properly routed to the requested information.

Computing system 100 may have more than one server 102 in a distributed structure with support from data centers that may be located anywhere around the world. These implementations may be communicatively linked and cross-platformed, so that a user on a mobile device (e.g., smartphone, tablet, etc.) or stationary device (e.g., desktop computer, landline telephone, etc.), may be provided with information relevant to their service request (e.g., electronic map displays, indicators which display travel times, routes, pricing information, profile information, settings information, level of familiarity, etc.). The term indicator as used herein is a means to transmit or display information related to services to a patient or to a service provider or both in a simple, fast, and convenient way. Features of the systems described herein can be implemented through computing devices that allow for method steps to be processed and output by a processor. Server 102 preferably coordinates user interfaces and interacts with database 108. Each user, depending on that user's role and needs, may be provided a different functionality.

Server 102, through a server interface, may receive patient input information, location information, and service request information to configure content, as well as the information from the caregiver (e.g., location information, limitation information, historical information, etc.). As discussed above, server 102 can send information to one or more computing devices through server interfaces, and information can be output to a display of the computing devices. Such content can include features which are region-specific if particularly relevant regional information exists, especially with respect to service request mapping or routing.

The service request information received through the server interface may be stored by the computing system 100 in database 108, and may include, for example: the status of service requests; the status of service request acceptances by caregivers; the reasons from caregivers for cancelling service requests; the histories associated with assigned service requests; operation logs of coordinators; etc. The content/timestamps of notifications and confirmation statuses may also be recorded in system logs, and this information can be checked by the administrator of computing system 100. It will be appreciated that this is not an exhaustive list of the operational service request information that the system may record.

The data stored in one or more databases 108 of computing system 100 can be continually updated with all user information discussed herein, and analyzed in accordance with the various methodologies discussed herein to enable efficient booking and dispatch of prescheduled home healthcare services. Every time computing system 100 gets an input/request from a patient, a caregiver, a coordinator, or other user, computing system 100 can first open a safe access channel with database(s)/database center and then send out query sentences through the access channel to a database management module. If a relational database is utilized, then the data tables may have one kind of relationships, such as one to many relationships, many to many relationships, and one to one relationships with other data table(s). Based on the relationships between data tables, the database(s) management module can exactly follow the query sentences and find the specific data table(s) by using ID(s), table names and column names of the tables with/without joining two or more data tables together. If a non-relational database is utilized instead of data tables, with the data stored in key-value pairs, then the database management module can exactly follow the query sentences and find the specific data by using keys that query sentences provide.

Computing system 100 can access all information stored in the one or more databases 108. Database(s) 108 can store rules and procedures data, caregiver data, administrative data, group data, patient data, map component data, and any other data relevant to implementation of computing system 100. Rules and procedures data can include system price, promotion setting rules and procedures, as well as rules and procedures for indicators, referrals, payments, service requests, system management, system log, system analysis and optimization, etc. The map component data can store map data for service requests that are identified by the GPS and LBS. The GPS and LBS data can determine the location of the computing devices in different ways, such as, for example, through receiving location-based resources. Caregiver data can include caregivers' profiles, such as personal data including a photo of the caregiver and years of his/her driving experience (e.g., if the patient needs transportation services), gender, country of origin, and language abilities.

The comprehensive database 108 may also store the details of service requests for each particular caregiver for future reference. Database 108 can also include data in the caregiver's profile that may include such information as a caregiver's favorite list and blacklist, limitations related to zip codes, time, location, and price, as well as service data and records. Database 108 can further include administrative data comprising prices and rates, system data such as contact and FAQ information, and registration details regarding patients and caregivers, such as, for example, billing information or other relevant information relating to administering the prescheduling service application. By way of example, the registration details can include how long users have been registered with the system or how frequently they use the prescheduling or on-demand service application. Other stored information can include service rules, procedures, and prices, as well as procedures for caregivers' and patients' settings. For example, database 108 may store billing information or other information related to administering on-demand service applications for computing system 100. Group data can include base data, company data, group of individuals data, or data related to vendors. Patient data can comprise patients' profiles including personal data, patients' favorite caregiver lists, patients' caregiver blacklist, patients' preferences, service requests data, and records. One of ordinary skill in the art will appreciate that database 108 can sync dynamically so that whenever changes or updates in data blocks are made, server 102 and database 108 dynamically update the data accordingly to reflect the latest changes. Additionally, at least one backup database may be utilized to back up a primary one of databases 108 in case of data loss in the primary database 108. One of ordinary skill in the art will appreciate that database 108 components may vary from the ones depicted herein.

Alternatively, computing system 100 may use a set of databases or data storage mediums to provide and maintain a prescheduled service application in order to dispatch a compatible caregiver based on a patient's preferences and needs. Databases 108 may contain several data categories or groupings. Sections of database 108 may be independent or synchronized in order to retrieve information from both sections at the same time. Such data can include predetermined rules and procedures data, administrative data, patient data, caregiver data, group data comprising base data, company's data, or group of individuals' data, and other types of data such as data relating to user types. All historical information may be categorized and stored in and retrieved from database 108. Historical data can be tracked in part by assigning a tracking number, service ID number or service ID corresponding to each service request to help computing system 100 refer back to the service request. Information categorized with this identification may include the type of service request, who requested and carried it out, where it took place (zip code, borough, county, city, state, etc.), what the route was, the cost of the service request, the services provided, when and how payment for the service(s) took place, and whether either party was added to a favorite list or a blacklist. All information regarding a patient's or caregiver's preferences or limitations, pricing, and other customizable information can be stored within database 108.

Records of completed service requests can also be stored and maintained within database 108. Computing system 100 can automatically store records of historical data for any completed service requests in a comprehensive database. Database 108 may be dynamically updated as services are booked and completed. Database 108 may store an index of each service request that has been requested and completed, including the registration numbers or user identifications of patients and caregivers, which can be retrieved for reference if needed at any time.

The service request information stored in the database 108 may include, for example, a service request ID, relevant caregiver information, relevant patient information, requested visit location, actual visit location, requested drop-off location, actual drop-off location, visit start time, visit end time, distance, duration time, status, prices, insurance company, etc. Even if a patient does not have a smartphone or use the application which is in communication with the system, this will not adversely affect the functioning of the system as methods which circumvent a patient not having access to the system may be utilized. For example, a coordinator may update such patient on the status of his/her service request or on the location of the caregiver. The coordinator can provide the patient with the most current information, as the start button functionality discussed above allows a caregiver to instantly connect with the coordinator.

The relevant service information may include information such as first name, last name, username, email, password, phone number, date of birth, gender, country of origin, caregiver type (e.g., owner-operator), affiliated company name, affiliated base name, provide wheelchair, language, signature, and general profile. The relevant service information may also include insurance information such as liability status, insurance status, insurance provider, insurance start date, and insurance end date.

Any backup databases related to database 108 may also be updated accordingly to reflect the latest changes. Such information can be organized or structured in a manner allowing for effective sorting and retrieval. The information can be stored in a non-relational or unstructured manner. One of ordinary skill in the art will appreciate that numerous methods for providing, storing, and organizing data in database 108 or other data storage medium may be utilized in accordance with the exemplary embodiments of the present invention discussed herein. Additionally, it will be appreciated that at least one backup database may be utilized which backs up the primary database frequently in case any data is lost from the primary database.

Information stored in the database(s) can be used to generate various indicators (further discussed below) in the prescheduled service system 100. The indicator related icons, shapes, or other depictions can be stored in database 108. Based on the results of exploration and analysis of data in database 108, as well as icon, shape or other depiction assignment rules, the icons, shapes or other depictions can be displayed with other indicators to patients, caregivers, and coordinators accordingly. Using information maintained about the patients and the caregivers stored in database 108, the prescheduled service system 100 can provide relevant information to any prescheduled service application operatively associated therewith. Caregiver information may correspond to information about the available caregivers themselves, such as profile information about the caregivers, current location, or particular distances from the patient or estimated time periods before pick-up. The system may set a default location to be a caregiver's home address or the address identified on a caregiver's license or one preset by the caregiver. The caregivers are also able to input certain location and time preferences, where the time preferences may include times of day, days of week, weeks of year, etc. or any combination thereof. During prescheduling or at any time during each day, the system may utilize the time and location data, or may use geolocation technology to identify real-time location and time information of any caregiver in communication with the system. The caregivers may also be able to preset selected locations in which he/she prefers to begin his/her shift or day, the area in which he/she prefers to work for the day, and the location in which he/she prefers to end his/her day. For example, a caregiver who resides in Brooklyn but prefers to primarily work in Flushing, Queens, may wish for his/her first service request to be a service from Brooklyn to Queens. After working his/her shift in Flushing, Queens, the caregiver may additionally prefer to have his final service request be a service from Flushing, Queens back to Brooklyn.

Additionally, specific data such as times of the service requests and prices for the service requests may be used as a reference to find average prices for patients so that they may receive fair prices when booking services. Data which is entered and stored in database 108 can be subject to continual verification. Maintaining a comprehensive database 108 also allows for the accurate tracking of service requests that may be in dispute by a patient or a caregiver. Service request records may be retrieved in an efficient manner from database 108 in order for any claims to be resolved in a timely manner.

Additional data may be input into database 108, including, but not limited to, locations where patients traveled to, favorite lists or blacklists, locations, other transaction data and details, historical data, insurance policy expiration dates, inspection dates, caregivers' license expiration dates, or any combination thereof. This data may also include information relating to indicators and the display thereof. By way of example, the data may include the service requests that all patients or caregivers have completed in a certain area, such as one or more streets, zip codes, town, city, borough, county, state, or any other region defining feature, or how many times a patient and a caregiver have been paired by computing system 100.

It will be appreciated that computer program instructions used by the computing system 100 may include computer executable code. A variety of languages for expressing computer program instructions are possible, including without limitation, C, C++, Java, JavaScript, Python, assembly language, Lisp, and so on. Certain logical functions may need to be implemented to provide highly specific and tailored services for patients and caregivers for the technical challenges that need to be solved. These logical functions may be pre-programmed to accommodate various preferences and limitations, and may be exceedingly complex to carry out particular if-then scenarios. Rules may be established that identify certain parameters. In this manner, many complex conditions may be accounted for, and caregivers and patients can be filtered by chaining logical functions based on those filters. Various language features can be utilized, and programmed and implemented through programming languages such as Java. Such languages may include assembly languages, hardware description languages, database programming languages, functional programming languages, imperative programming languages, and so on. Computer program instructions can be stored, compiled, or interpreted to run on a computer, a programmable data processing apparatus, a heterogeneous combination of processor architectures, and so on.

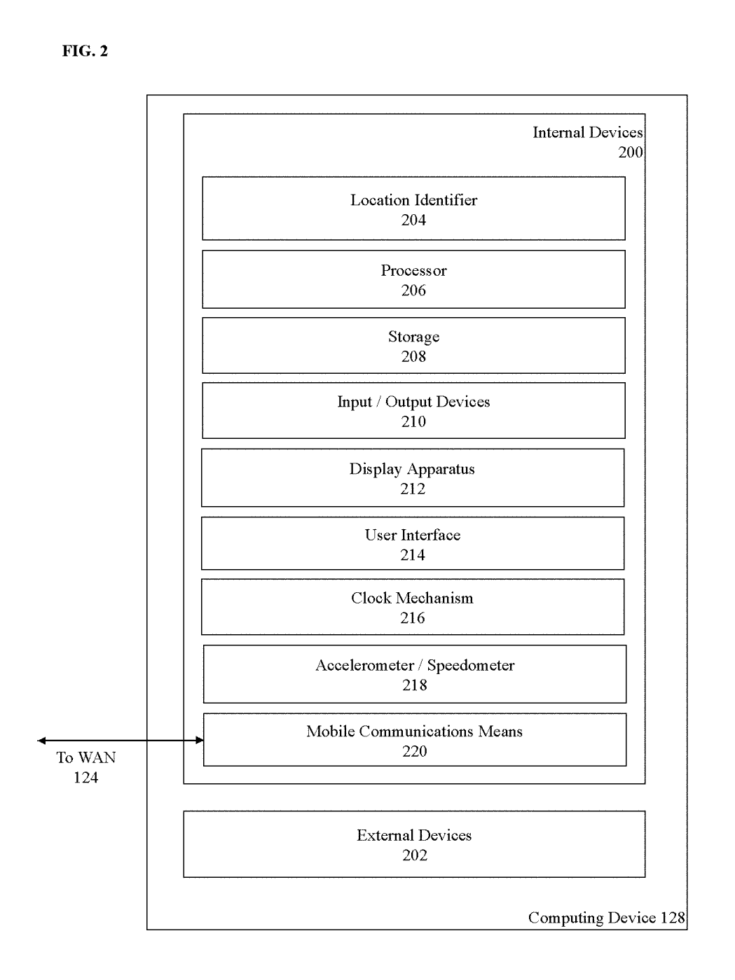

Turning now to FIG. 2, shown is a diagram illustrating various components of an exemplary embodiment of computing devices 128. As discussed above, computing devices 128 can be used by patients (e.g., via devices 130C1 . . . 130Cn) or caregivers (e.g., via devices 132D1 . . . 132Dn) or both, and may be in communication with various components, tangible or intangible, of computing system 100. Computing devices 128 may incorporate various internal devices 200 and external devices 202, and may utilize mobile communications devices 220 for receiving voice, text, and data for connecting to computing system 100 such as over a WAN 124, and location identifier 204 such as a Global Positioning System (GPS) receiver for identification of a present location. An application, a map component, map data, and location identifier 204, such as, for example, a GPS module or other circuitry for providing location-based services (LBS) data may be integrated into one or more of computing devices 128 for certain location identification functions. Location identifier 204 may identify a location of computing devices 128 in different ways, such as, for example, through receiving location-based resources. One of ordinary skill in the art will appreciate that there are numerous approaches for providing location identification and location based services. A GPS-enabled system or device allows tracking components to identify the location of computing devices 128. For example, location identifier 204 can be instantiated through processing received GPS data from location-based or geo-aware resources of computing devices 128. Additionally, location identifier 204 can receive GPS data from other applications or programs that operate on the computing device using one or more application program interfaces (APIs). The application can use location information to cause a user interface component to configure a user interface framework.

In preferred embodiments, a patient can access a patient module such as computing device 128 operatively associated with computing system 100 to input a service request which includes service details such as pick-up and drop-off locations as well as desired pick-up and visit end times. If, however, any entity requests a service which is deemed incompatible with the system (e.g., based on data received from location identifier 204 regarding the location of one or more caregivers and/or caregiver limitations in database 108), then a coordinator may be notified.

Computing system 100 may be configured to generate a notification to patient device 130 when a caregiver comes within a region that is a certain distance (e.g., one or two miles) of a patient visit location designated in a service request. Such location-based services, facilitated by location identifiers 204 in caregiver devices 132D1 . . . 132Dn, allow for efficient routing of caregivers based on point-to-point directions. Caregiver devices 132D1 . . . 132Dn may each include an interface component which provides location information gathered by location identifier 204, and passes such location information to an interface component on patient device 130C1 . . . 130Cn via WAN 124 and server 102. Caregiver devices 132D1 . . . 132Dn may also include radio-frequency identification (RFID) technology, or other similar identification device or means.

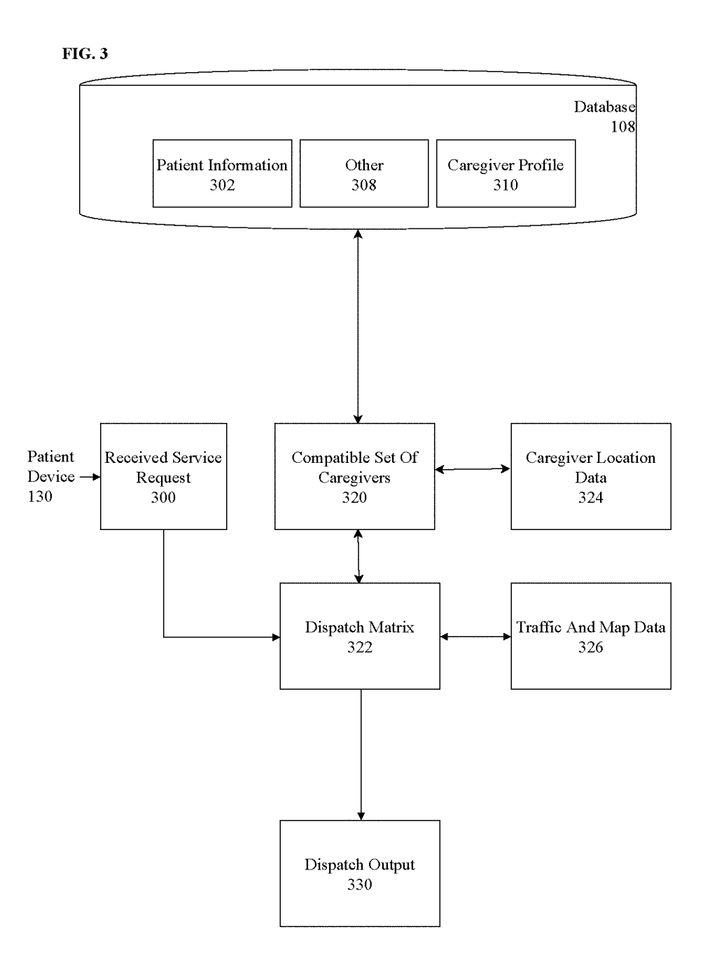

The location identifier 204 can include a GPS-enabled system or device whose tracking components identify the location of patients who are making service requests and caregivers who are looking to provide service. Computing system 100 may include an application manager which, based on a patient's current location or service location, may cause a region-specific patient interface feature to be output by a patient interface component on display apparatus 212. A region specific to a patient can include the patient's current location or a service location in which the patient wishes to preschedule service. The region may be identified by zip code, city name, metropolitan area name, etc., in which computing devices 128 are currently located, and may be an area having a certain distance or radius from the patient's current location (e.g., one mile, five miles, etc.), or may be an area specifically partitioned from other areas. Region-specific information about the prescheduled service may be provided in part by a system which supplies caregiver location data 324 (FIG. 3). It will be appreciated that use of location related preferences or limitations may depend in part on GPS-enabled devices. By cross-referencing data, the prescheduled service systems described herein can identify specific locations (e.g., stores, restaurants, apartment complexes, venues, street addresses, etc.) proximate to and/or located at an identified location, and provide this specific location information as location data (e.g., as part of the traffic and map data 326).

Processor 206 is preferably provided for executing software or a set of instructions on computing devices 128. Computing devices 128 may also contain storage 208 such as random-access memory (RAM) or flash storage. Input/output (I/O) devices 210 can be used to connect computing devices 128 to other system implements, depending on the available functionalities of computing devices 128. For example, a caregiver may use an in-vehicle navigation system, which might not have a camera, while a smartphone may have a built-in camera. In this situation, a camera may be included as an input for the in-vehicle navigation system. Other I/O devices 210 may include a scanner, a microphone, and/or a speaker. Computing device 128 may also include display apparatus 212 to receive and display to a user notifications and/or other data received from computing system 100. Display apparatus 212 may, for example, be an electronic touchscreen display configured to provide user interface 214 in accordance with various methodologies of the invention. Computing devices 128 may also utilize internal clock mechanism 216 to determine the present time. Accelerometer/speedometer 218 can also be provided as part of or in communication with computing devices 128 to measure speed, acceleration, directional changes, etc.

User interface 214 displays various content based on user selections and preferences. It will be appreciated that one or more components of computing devices 128 may be combined to provide user features specific to user selections and user locations. These selections can be displayed to the user, and the user can utilize user interface 214 to interact with displays of certain information. For instance, user interface 214 can correspond to a program downloaded onto a smartphone or other portable computer device such as a tablet computer or personal digital assistant (PDA). A user can download and install the application on one or more computing devices 128 and register with the system. Pre-programmed features of computing devices 128 may be utilized based on certain protocols or methods of integration of basic components, such as servers, databases 108, mobile end applications, web portals, network settings, etc.

All types of users can be registered and entered within the system for the purpose of activity tracking. Registration may be performed through means such as assigning a user ID to each user such that system functionalities can only be accessed when a user ID is entered. Additionally, the device the user employs to access the system may be tracked by IP address, and system activity may be tracked with timestamps or similar means and stored in database 108. In this manner, a system administrator can track not only who is accessing the system, but also from which device, the location of the device, and the time of such access. Such capabilities allow coordinators to track activity, and if an error occurs, such as entry of a wrong address on a service request, then the cause of the error can be easily diagnosed and addressed. It is currently a drawback in the industry that such errors cannot be detected and located, especially when a coordinator does not want to admit the error. It will be appreciated that such functionality also provides a means for added security. Any service request entered from an unauthorized computer can be ignored. Unless computing device 128 is given access permission, it cannot access certain functionalities restricted to registered users. Allowing a dispatch system to be run in part by coordinators allows for increased flexibility and executive function if necessary, as exceptional situations can arise which require human judgement.

User interface 214 can be, for example, a homepage, access to a dispatch portal (for caregivers), a service requesting module (for patients), an access interface to database 108, or a way for users to access one or a combination of any of the features described herein. The system can retrieve a user's information and other data that is stored in database 108, which may be provided locally and/or remotely. One of ordinary skill in the art will appreciate that numerous additional user interfaces are contemplated for use with or in place of user interface 214.

Patient devices 130C1 . . . 130Cn may each utilize a patient interface to display various indicators on maps showing geographic information. Each indicator can signify, for example, differing patient information or patient inputs received by computing system 100 from the patient, a vendor, or any application which takes a prescheduled service request from the patient. Patient devices 130C1 . . . 130Cn can also each contain application features adapted to dynamically synchronize content based on patient selections provided via a patient input. User interfaces 214 on patient computing devices 130C1 . . . 130Cn can include, but are not limited to, a home page patient interface, a service request panel used for patients to identify the details of service requests, preference details, etc., a summary patient interface, a location search patient interface, a confirmation page interface, or a combination of any of these features. By way of example, if a patient's current location is different from an originally requested visit (e.g., service) location, then the patient can manually preschedule a new visit location that is different from the current location stored in computing device 128 or computing system 100.

A start button functionality can be provided as part of computing device 128 on, for example, one or more of caregiver devices 132D1 . . . 132Dn. The display of one or more mobile caregiver device 132D1 . . . 132Dn may display relevant information to a caregiver about the service queue, starting with the next service in the queue, where the details of that service are shown, such as the visit location and visit start time along with the destination and the scheduled visit end time. The caregiver can then click ‘Start’ (e.g., a physical push button or via a touch screen interface on caregiver device 132D) in order to let a dispatch or administrator know that he/she has begun the service and is on the way. The display on the mobile device may also show a list of the remaining services in the queue with limited details which may be expanded or viewed later. This Start button functionality helps address current drawbacks in communication between various parties as coordinators can easily identify which service requests are underway. Additionally, it provides a means by which a patient who has scheduled a second leg can let a caregiver know the status of the related appointment. In a traditional dispatch system run by telephone, the current location of caregivers and the status of second leg patients may be unknown. As a result, coordinators have no choice but to operate by guesswork if they cannot easily reach a caregiver or patient while coordinating a service request. When a caregiver presses a start button at the beginning of each leg of a service request, the system can record the status in database 108. Such functionality will also make tracking easier if the duties of a service request are handled by more than one caregiver.

It will be appreciated that the systems and methodologies disclosed herein provide functionalities which allow for a much smoother and more efficient process overall than those of conventional dispatch service methods. Pressing the Start button may trigger a series of events which affect the dispatch backend system, and that series of events may be carried out with the help of various software and hardware implements. Pressing the Start button may, for example, cause the caregiver's location to be relayed to a third-party mapping and navigation service, which may configure various routing options and the ETA for the caregiver on each route based on the caregiver's current speed and the distance associated with each route. This information may be relayed to a dispatch, a patient, and back to the caregiver, and may be provided in real-time.

The user interface may also include a Start button which triggers a series of events in database 108 regarding data storage, where the geolocation of the caregiver is tracked by location identifier 204 as part of the records associated with a service request, the patient, the caregiver, etc. Without this Start button functionality, GPS devices may still be able to detect a caregiver's current location and heading. However, when providing prescheduled services, the caregivers will have a long list of scheduled services, and without a caregiver actually confirming that he/she is underway to pick-up a particular patient, there is no way to be sure that the location which the caregiver seems to be heading towards is the patient's visit location. As a result, the Start button is a powerful feature that can provide coordinators and other parties with an instant update on the caregiver's real-time status.

In the healthcare industry, a clinic operator such as an employee of the clinic may be able to manage (e.g., search/add/delete/modify) appointments affiliated with the clinic, and by using the functionality of a start button, notify other employees of the clinic and patients of any changes. For example, the clinic operator or a patient may be able to notify other users that an appointment has begun by pressing the start button on their end. If, for instance, the appointment began later than scheduled, the system may be able to make any necessary changes, such as shifting an appointment to a different time or notifying patients of how the changes will impact their appointments. Additionally, a caregiver who has been assigned to pick-up the patient at the end of the appointment may get a real-time notification about the status of the patient, such as “Checked In,” “Seeing Doctor,” “Almost Finished,” “Finished,” etc., which allows a caregiver to be ready for any changes in the scheduled visit start time.

Patient devices 130C1 . . . 130Cn may be configured to allow a patient to manually supply location inputs by entering an address (e.g., street number, street name, city, state, etc.) or by manipulating and moving a service location graphic/icon on an electronic map display on a portion of a patient interface. In response to such patient selection, the prescheduled service application running on one or more of patient devices 130C1 . . . 130Cn can provide the location data to computing system 100.

Once the start button is engaged, computing system 100 can calculate an estimated time of arrival (ETA). A caregiver may be provided with potential jobs through this interface, where queries can be displayed temporarily for the dispatch of a service. A caregiver module can facilitate enabling the interface on a mobile device which the caregiver has. In the event that a patient cannot sign for a service, computing system 100 may depend on the caregiver to collect a signature on his/her phone from a patient.

In preferred embodiments, system 100 dynamically updates and stores any changes to a service request before or during the start thereof, or any updates on the status of the service request, and displays these changes in real time, both in a web portal for the coordinator, and in a caregiver interface on caregiver device 132 associated with the caregiver assigned to the service request. For example, if a patient cancels a service request or needs to change the visit start time or location, the patient can enter this information into system 100 via patient device 130. The new information is stored in database 108. The web portal of the coordinator updates, and a notification of the change is immediately sent to the caregiver associated with the service request via caregiver device 132. The caregiver can then preferably access the same information about the service request displayed in the web portal of the coordinator. Additionally, in preferred embodiments, any new information about the patient (e.g., phone number, email address, a change to preferences, etc.) entered prior to the service request can be communicated to the web portal of the coordinator and the user interface of caregiver device 132 of the caregiver assigned to the service request. Preferably, only relevant caregiver devices are updated with new patient information (e.g., caregiver devices associated with caregivers involved with the patient's service requests).

When a caregiver presses the start button at the beginning of each service or leg of a service request, the service status can be updated in the web portal of the coordinator and the patient device 130 of the patient. It will be appreciated that a patient can thus view an estimate of the arrival time of his/her caregiver in advance thereof. Such functionality will diminish or eliminate the workload of a coordinator as he/she will not need to field phone calls regarding changes to service, and will not have to call caregivers. Those skilled in the art will appreciate that these functions are merely examples, and that other functionalities of the caregiver's interface may be utilized.

External devices 202 (i.e., additional mobile devices, tablets, laptops, or other computing devices) can connect to computing devices 128 through a wired or wireless connection. It will be appreciated that these external devices 202 may include any device that can provide additional or enhanced functionalities to computing devices 128, whether computing devices 128 is a mobile device such as a tablet or smartphone, an in-vehicle navigation system, or another type of computing device.

It will be appreciated that computing system 100 can integrate different functionalities specific to various industries, including the NEMT industry. It is a conventional practice in the NEMT industry to receive service requests from brokers who request that services be provided at very specific times between two locations for which addresses are specified. These specific stipulations are understandably meant to combat fraud and make sure that service requests are completed properly. However, for caregivers in this industry, adhering to the exact timing and addresses specified is not always easy. Unexpected traffic congestion or construction prevent caregivers from arriving on time to both the pick-up and drop-off locations. Dropping a patient off at the exact address as requested may not be possible because of parking regulations, pick-up and drop-off rules, standing prohibitions, or other applicable laws or fines. The caregiver may need to wait a certain amount of time or complete drop-offs down the block or around the corner where stopping and drop-off is appropriate and legal.

The systems and methods disclosed herein may also be used in conjunction with the systems and methods disclosed in U.S. patent application Ser. No. 15/474,685, filed Mar. 30, 2017, entitled “System and Method for Geo-Aware Transportation Billing Verification,” the entire disclosure of which is hereby incorporated by reference herein. The bona fide nature of these billing adjustments can be confirmed by tracking a caregiver's geolocation using location identifier 204 and assigning a timestamp at the time the caregiver arrives at the patient's home and begins service or picks up the patient or at the time the caregiver leaves the patient's home or drops off the patient to make sure that the timing and/or location based attributes of services provided are within a predetermined field of acceptability. If a service request falls outside of such predetermined field of acceptability, then an inquiry may be opened to investigate the reasons for such deviation. In this manner, billing clerks can save valuable time, caregivers do not have to risk being fined, and brokers know that the service requests they booked were completed in good faith.

Referring now to FIG. 3, it will be appreciated that dispatch systems are tasked with receiving, organizing, coordinating, and dispatching a large volume of service requests. Equitably and efficiently prescheduling each caregiver with a patient can be challenging. When services are prescheduled, patients can be demanding with regard to preferences when selecting a caregiver to dispatch. For example, a Spanish-speaking patient may not be happy with a caregiver who cannot speak Spanish. The present invention may sort service requests based on patient preferences, and give priority to certain caregivers based on a variety of factors.

FIG. 3 is a diagram illustrating an exemplary dispatch operation incorporating an exemplary dispatch matrix in accordance with various embodiments of the invention. Service request 300 is received by computing system 100 and a caregiver is dispatched based on dispatch matrix 322 to complete service request 300. Information from service request 300, which a patient can submit through one of patient devices 130C1 . . . 130Cn (or by a vendor on behalf of a patient through vendor device 126), is included in a generated dispatch matrix 322. It will be appreciated that a patient may also enter or submit service request 300 using a traditional telephone. Once a particular patient or vendor submits service request 300, processor 104 executes instructions for retrieving relevant data from database 108 relating to a particular patient's information 302. Data may preferably be stored categorically within database 108 and be updated dynamically to ensure the most current and up-to-date information is used in the prescheduling process.

Patient information 302 may include any information associated with the patient, including, for example, the patient's transportation needs, one or more patient preferences, a patient favorites list, a patient blacklist, patient contact information, patient billing information, etc. Patient information 302 can also include the patient's home address, the patient's place of business, the patient's preferences, historical information such as information about previous locations for which a patient requested service, recommended points of interest, etc. When a patient selects one of the entries of a recommended point of interest as a current location and/or visit location, the system may retrieve various types of historical and current caregiver location data 324. As shown, computing system 100 can additionally retrieve caregiver profile information 310 from database 108. Caregiver profile information 310 can include caregiver service limitations, caregiver history and/or service history, caregiver favorites list, caregiver blacklist, etc. It will be appreciated that database 108 can store patient information 302 and caregiver profile information 310 for a plurality of patients and caregivers, as well as any other data 308 desired.

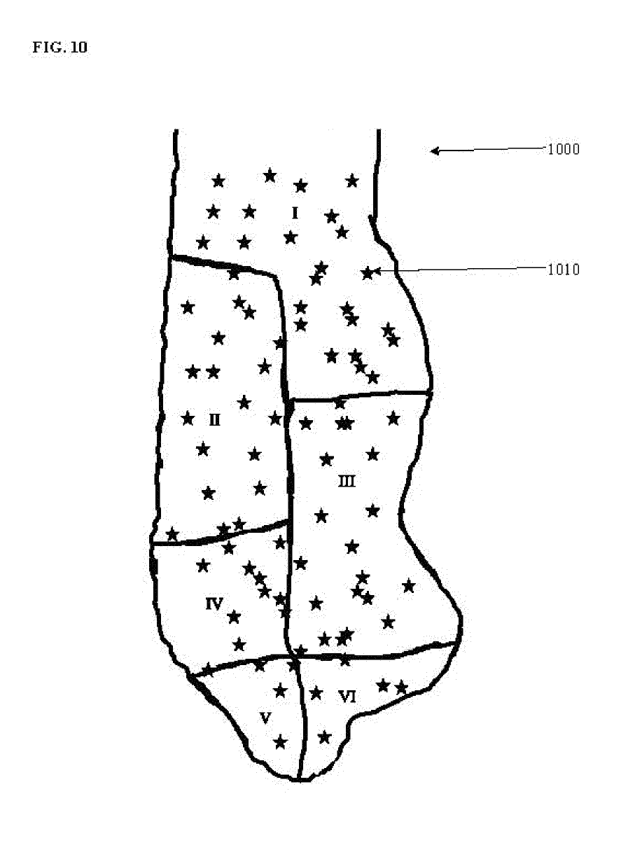

A caregiver may preset various limitations which are meant to limit the scope of service requests that the caregiver would like to provide, and which are taken into consideration when identifying matching caregivers for a patient's service request. For example, a caregiver's limitation that relates to distance from the caregiver's current location to the visit location may prevent computing system 100 from displaying a caregiver for a particular service request whose current distance from the requested visit location exceeds the distance that this caregiver has preset as location or distance limitation. A caregiver may prioritize the various preset limitations in order to resolve potential conflicts when they arise. Additionally, as further discussed below with respect to FIG. 10, the preset caregiver limitation on distance to a visit location can limit the unavailable service requests 1010 displayed on an electronic map display 1000 to the caregiver. Similarly, a caregiver may not be displayed to the patient as a potential option in the event that the prescheduling service is switched to an on-demand service.

The historical data retrieved by computing system 100 for each caregiver can include information on whether the caregiver has been previously assigned by the particular coordinator handling the present batch or auto-dispatched by computing system 100, whether the caregiver has reserved any particular zip codes where he/she prefers to provide service, the number of times the caregiver has previously handled service requests along the route of the duties of the service request (directly or indirectly), and the caregiver's rating (e.g., user rating, number of times he/she has cancelled services and/or been rejected or blacklisted by patients, etc.). Each of these factors can be assigned a particular weighting by computing system 100 in accordance with one or more predetermined rules. The historical data can also include a caregiver ID number or name, home address, a preferred service location or geographic region, preferred working hours, a replacement list, a favorites list, a blacklist, a scheduled service list, and an accepted service list. These other factors relating to the patient's preferences, the caregiver's limitations, and other information associated with the patients and caregivers may be utilized when assigning caregivers to service requests or duties thereof. Various algorithms and weighted factors may be utilized in making caregiver assignments in accordance with the systems and methodologies discussed herein. It will also be appreciated that if any caregivers not in an originally retrieved potential set of caregivers identified for a batch of service requests (further discussed below) are in close proximity to the visit location of a service request and available, then computing system 100 can assign one of such caregivers provided such caregiver is compatible with the patient (e.g., not on the patient's blacklist, not having the patient on his/her blacklist, and not precluded by any absolute mismatches between the patient's preferences and the caregiver's limitations).

Based on patient information 302 and caregiver profile information 310, dispatch matrix 322 can be implemented to preclude one or more caregivers who cannot provide service for service request 300 because of one or more aspects of service request 300 and/or who do not wish to provide service to this particular patient, based on a set of rules relating to compatibility. If, for example, a caregiver's limitations (included in caregiver profile 310) conflict in some manner with service request 300 (e.g., if service request 300 includes a service from Brooklyn to Manhattan, but caregiver profile information 310 indicates that the caregiver has opted not to provide service in Manhattan, or if service request 300 includes a pick-up at 7 AM, but caregiver profile information 310 indicates that the caregiver wants his/her earliest time for picking up patients to be 8 AM), then such caregivers can be precluded from inclusion within a compatible set of caregivers 320 generated by computing system 100.

Caregiver location data 324 and/or traffic and map data 326 can be retrieved by computing system 100 and factored into the system's determination of whether a particular caregiver is sufficiently compatible for inclusion in the compatible set of caregivers 320. Caregiver location data 324 can include a preset and/or relatively current location of the caregiver or the location where the caregiver is scheduled to be at a certain time. Traffic and/or map data 326 can include geographic, traffic, and travel time information associated with particular areas. It will be appreciated that database 108 can include caregiver location data 324 for a plurality of caregivers and traffic and map data 326 for a wide array of geographic areas.

Computing system 100 can generate a compatible set of caregivers 320 and dispatch matrix 322 from service request 300, patient information 302, caregiver profiles 310, and/or caregiver location data 324 by using a processing function in which different variables having different priorities are processed. For example, received service request 300 may include numerous details, such as timing information and locations to which one or more patients want to travel. Caregiver location data 324 and traffic/map data 326 (e.g., routes, traffic information including traffic patterns, congestion, etc.) can additionally or alternatively be factored into the calculations. In addition, a caregiver priority can be established which includes a certain weight assigned to a caregiver indicative of how well a particular caregiver matches service request 300, the patient's preferences, and the patient favorites list, and/or the feasibility of the caregiver being able to service the request given where he/she is expected to be at the time of pick-up (caregiver location data 324), and the expected traffic patterns (traffic and map data 326).

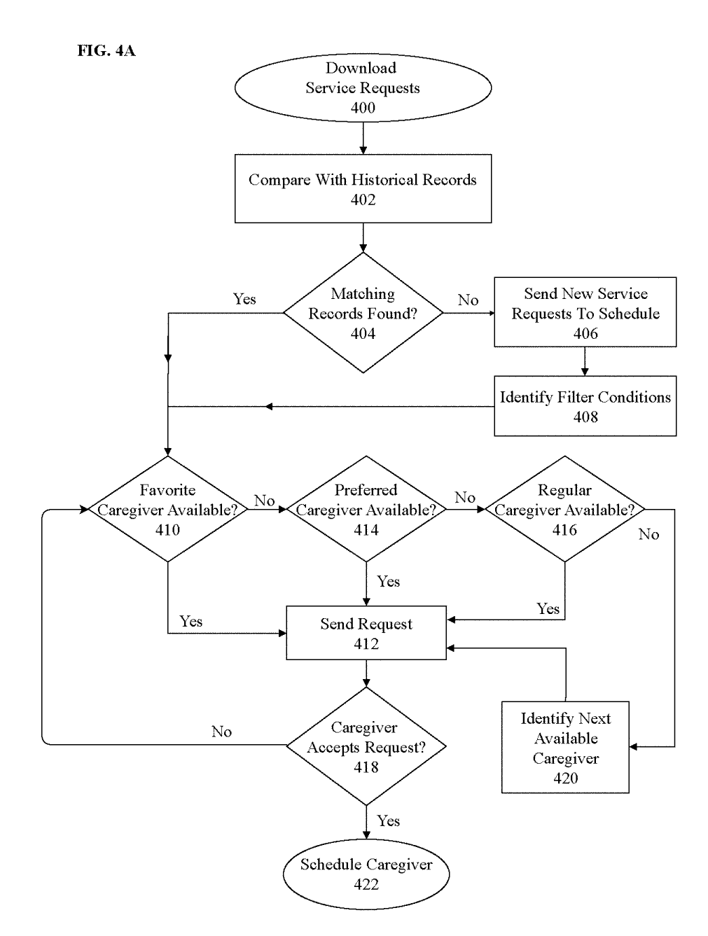

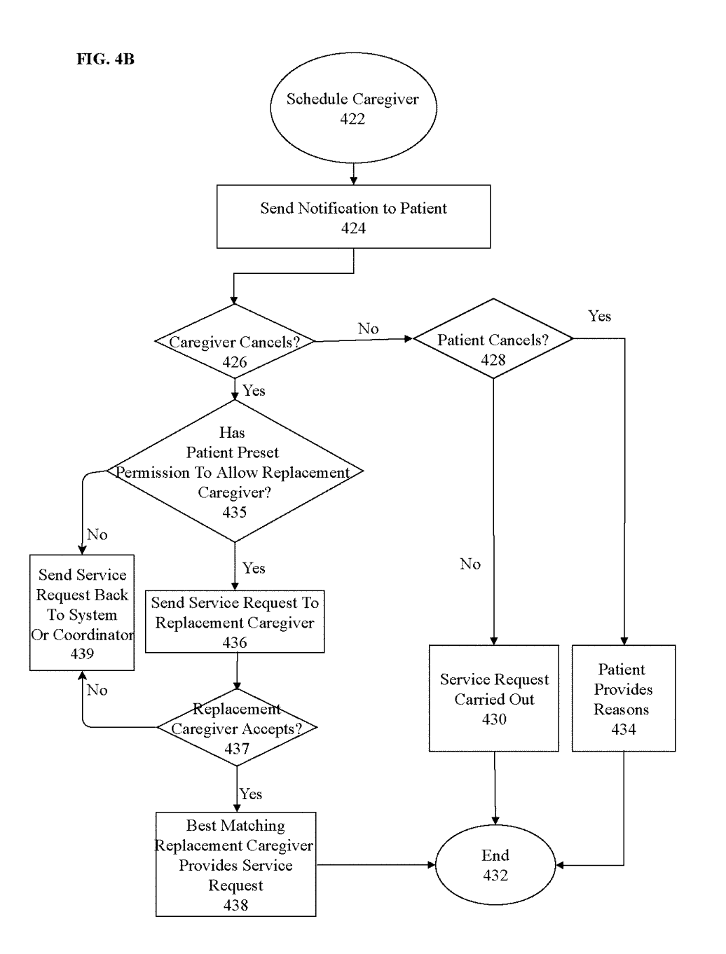

By way of example, two of the compatible caregivers 320 who are both familiar with a given patient, the route of service request 300 and who have no service limitations which interfere with service request 300 may be assigned the same weighted priority for these factors. However, if one of the two caregivers is on the patient's favorites list while the other is not, then the caregiver on the favorites list may be prioritized over the caregiver not on the favorites list, and thus assigned a greater caregiver priority. Based on the various caregiver priorities established in dispatch matrix 322 for the compatible set of caregivers 320, computing system 100 creates dispatch output 330. Dispatch output 330 can include a display of a selection of potential caregivers, and can be automatically sent to the patient by computing system 100, or reviewed by, for example, a third-party administrator 134 operating computing system 100, and then sent to the patient. Dispatch output 330 may be a subset of the compatible set of caregivers 320 or the entire compatible set of caregivers 320 depending on how many caregivers make the cut into the compatible set 320 and depending on how many options for caregivers the patient wants to view. The patient can then select a caregiver from the list of caregivers included in dispatch output 330. The caregiver selected by the patient may then be given the option to accept or may be automatically scheduled (further discussed below with respect to FIG. 4B).

If a caregiver accepts the service requests, then he/she may see the status of the accepted service requests on his/her service request list, marked “Accepted.” The dispatch system may forward a batch of service requests to a particular caregiver based on the preferences established by the caregiver and the patient for the service request, as well as any time, day, and location limitations established by the caregivers. Upon receipt of the batch or group of service requests sent to the caregiver by the system, each caregiver may accept or reject service requests at his/her discretion. However, a caregiver's preferences (i.e., favorite list, blacklist, location served, etc.) may be modified based on the caregiver's decisions in accepting or rejecting certain service requests. That is, in certain embodiments, if a caregiver rejects a service request from a favorite patient, then he/she may be removed from the favorite list of that patient. Similarly, if a caregiver rejects, over time, a number of service requests from a particular patient, then, in certain embodiments, he/she may be added to a blacklist of that patient. Once a service request is accepted by a caregiver, in the event that such caregiver is not going to be able to arrive for a selected service request within the predetermined acceptable time period, then the system enables the caregiver to transfer the service request to a replacement caregiver, or to notify the system that he/she is unable to complete the service request so that the system can reassign the service request or place the service request into the job pool.

The caregiver may then select one or more service requests. After the caregiver selects more than one service request and begins to act on the first service request, a list of all of the selected service requests may be presented to the caregiver via one of caregiver devices 132D1 . . . 132Dn for reference, and the caregiver may update the status of each service request. For example, each service request may have buttons for “Navigation”, “Arrived at Visit location”, “Begin Service”, “Arrived at Drop-off Location”, etc. If there is more than one service request that the caregiver is handling at the same time, then the caregiver may be able to decide pick-up and drop-off in sequence. A signature function may be added to the system for patients to e-sign before or after the service request has been finished.

Each of caregiver devices 132D1 . . . 132Dn may also include a lockout screen that blocks other functions such that the only item appearing is a notification which queries the caregiver whether or not to confirm a service. Such notification may show the details of the service, such as visit location, visit start time, destination, and visit end time, but without any other interactive feature other than a “Yes” or “No” query for service confirmation.

Prescheduled service requests may be shown on a list with each having the caption “Assigned” or “Unassigned” over a status column. Caregivers may be given two options: accept or reject. If a caregiver accepts the service request, then the service request may show “Accepted” in the status column of the service requests list. If a caregiver rejects the service request, then the service request may show “Rejected” in the status column, and the pre-scheduled caregiver's name may be shown for reference. Service requests which are deleted from the updated list or version based on records in database 108, or ones which are marked “Cancelled” in the updated version, may be stored as cancelled service requests in computing system 100. If certain cancelled service requests were originally assigned to caregivers and started, then the system can mark or store them as “Cancelled After Start,” and the name of the caregiver may additionally be recorded for further reference. If new service requests are received which did not appear in previous service request schedule updates, then such new service requests may be displayed to the coordinator, and may be assigned a high priority in order to remind the coordinators to find a caregiver and dispatch those service requests as soon as possible. If coordinators receive new service requests by, for example, phone, email, text message, etc., then these could be entered manually into the service requests list, and subsequently assigned to caregivers who can either accept or reject them.

The system can determine compatibility between a patient and a caregiver for a service request by comparing various factors relating to each user's limitations and preferences. For example, if a caregiver has not indicated Mandarin language abilities and a patient prefers a Mandarin-speaking caregiver for all of his/her potential caregivers, then all caregivers who do not speak Mandarin can be deemed incompatible by the system. With regard to time limitations, a caregiver's time limitations can be compared to the estimated travel time to complete the patient's service request, including the time for which the caregiver may be underway from traveling to the visit location to the drop-off location to the return location, if applicable. If the estimated travel time is beyond the caregiver's time limitations (e.g., the caregiver would be unable to get to where he/she needs to be by a certain time), then the caregiver may be deemed incompatible for that particular service request. The system can also compare the caregiver's location limitations with the route of the service request. If a caregiver has indicated that he/she will not go to certain areas, then in situations where the patient's pick-up or drop-off location involves at least one of those areas, the caregiver can be deemed incompatible. Each patient can customize which, if any, factors or attributes is/are relevant to his/her compatibility preferences for being matched with caregivers for prescheduled services, and the degree/extent to which each attribute is relevant. The system can be configured to make a weighted calculation based on the patient's preset priority levels for various preferences pertaining to the caregiver, the caregiver's vehicle if he/she uses one, the caregiver's capabilities, and/or whether or not a particular caregiver has or offers such preferences.

It will be appreciated that location limitations may greatly affect where a caregiver eventually provides services, and that the exemplary embodiments discussed herein allow for an efficient dispatch system that can use different defined geographic regions as a factor when scheduling service requests. These regions may be defined by country to smaller divisions such as state, county or borough, neighborhood, and even zip code. The system can be configured to identify where service requests take place, particularly the start locations and end locations, as these relate to the times at which the service requests are scheduled, and can group them by region. It will be appreciated that such groupings using the information in database 108 will provide improved efficiency over standard practices of dispatching randomly. For example, in a large city such as New York, a caregiver may be dispatched at random times to random locations. In the NEMT industry, a caregiver may, for example, drop-off a patient in the Bronx and immediately be dispatched to visit the home of and provide service to another patient in Far Rockaway, Queens. Although both of these locations are within the New York metro area, they are relatively far apart geographically. In addition, after factoring in traffic and navigation difficulties common to New York, the travel time between these two locations can easily be upward of an hour or more. By grouping service requests according to geographic area and tracking caregivers, dispatching can be performed more efficiently, enabling caregivers to complete more service requests in less time. It will also be appreciated that such groupings will make it easier for coordinators to preschedule service requests with much higher efficiency.

It will be appreciated that the systems and processes described herein will allow for prescheduling of service requests and caregiver assignments, and handling on-demand service requests which require immediate real-time service or which require last minute changes to existing service requests, whether such prescheduling is for same day service, for service just hours in advance, or for service days or weeks in advance. By storing patient information, caregiver profile information, caregiver location data, and traffic map data in the manner discussed above, computing system 100 is able to quickly eliminate a number of caregivers in database 108, and match a service request to a select list of caregivers who are deemed most compatible with the patient and his/her service request, and based on the caregiver's location or expected location. It will be appreciated that such processing allows computing system 100 to operate more efficiently because it can quickly focus in on more compatible and geographically close caregivers.

Turning now to FIG. 4A, shown is a flowchart illustrating an exemplary dispatch operation in accordance with an embodiment of the invention. The process starts with computing system 100 downloading or otherwise receiving service requests 300 (Step 400) which may have been uploaded by a vendor and/or submitted by a patient. Computing system 100 may download service requests 300 automatically based on programmatic steps, or a coordinator can manually download the service requests by, for example, clicking a download link in a web portal or refreshing a pending requests page. Computing system 100 may be configured to allow for several ways in which the service requests can be populated and updated to reflect any changes. For example, computing system 100 may be configured to access relevant vendor web portals or an application program interface (API) to download the list of service requests. In order to ensure that the service request list reflects the most recent changes, computing system 100 may perform such downloads repeatedly at certain intervals (e.g., every 5 minutes, every 15 minutes, every 30 minutes, etc.). Vendors may also supply and edit service requests through a vendor module (e.g., through vendor device 126). In either case, computing system 100 then processes the updated list of service requests, and compares them with past service requests based on historical records stored within database 108 to determine whether there are similarities (Step 402).

Computing system 100 then determines whether its details match a record of any previous service requests stored in database 108 (Step 404), such as, for example, a matching patient name, a visit location, and/or a drop-off location. If no match is found (No, Step 404), then the service request 300 is sent to dispatch for new processing (Step 406). Before service request 300 is dispatched, however, the system identifies any filter conditions (408) (e.g., level of familiarity with the patient and/or service needs of the patient, level of familiarity with the route to get to the patient or to take the patient from his/her home to another location, prior number of service requests performed for the patient, etc.) that service request 300 may have, and accommodates those filter conditions by, for example, comparing them to relevant portions of the caregiver's limitations in database 108, and precluding certain caregiver's from being selected to complete the service request (e.g., if the service request requires that the caregiver be fluent in a particular language, but the caregiver does not speak the particular language). If matching records are found (Yes, Step 404) or after new service request records have been processed (Steps 406/408), computing system 100 searches the records in database 108 to determine whether a favorite caregiver of a patient for a service request is available to carry out the service request 300 (Step 410).

If a favorite caregiver is determined to be available (Yes, Step 410), then computing system 100 sends service request 300 to the favorite caregiver for acceptance (Step 412). If no favorite caregiver is determined to be available (No, Step 410), then computing system 100 searches database 108 to determine if a preferred caregiver is available (Step 414). If a preferred caregiver is available (Yes, Step 414), then computing system 100 sends service request 300 to that preferred caregiver for acceptance (Step 412). If no favorite or preferred caregiver are found to be available (No, Step 414), then computing system 100 identifies whether a regular caregiver is available (Step 416). If a regular caregiver is available (Yes, Step 416), then computing system 100 sends the service request to the regular caregiver for acceptance (Step 412). If no regular caregiver is available (No, Step 416), then computing system 100 continues to monitor all caregivers for the next available caregiver to be identified and scheduled (Step 420). Once an available caregiver is identified, service request 300 can be sent to the identified available caregiver (Step 412).

If the first regular caregiver to whom service request 300 is sent at Step 412 does not accept the service request, then the system can repeat the process until another caregiver is identified (Step 420). When a caregiver receives service request 300 on his/her caregiver device 132D1 . . . 132Dn, that caregiver has an option to either accept or reject the request (Step 418). If the caregiver accepts service request 300 (Yes, Step 418), then that caregiver is scheduled to complete the service request (Step 422). If, however, the caregiver rejects service request 300 (No, Step 418), then computing system 100 cycles back to step 410 and checks again whether there are any favorite caregivers available. If no favorite, preferred, or regular caregivers are available, then after a certain amount of time has elapsed, the coordinator may need to be notified so he/she can go through the process of identifying another caregiver (Step 420).

A caregiver may make additions to the caregiver's favorite patient list and/or the caregiver's patient blacklist after completing a service request. For example, the caregiver may be queried as to whether he/she was satisfied with the patient. If the caregiver was not happy with the patient, then the caregiver may add the patient to the caregiver's patient blacklist, in which case the caregiver and the patient will not be matched together for future service requests. If the caregiver is neutral on how he/she feels about the patient, then the caregiver need not take any action, and the patient will not be added to the caregiver's favorite patient list or the caregiver's patient blacklist. If the caregiver is satisfied with the patient, then the caregiver can send the patient a request to authorize addition of the caregiver to the caregiver's favorite patient list. In such embodiments, the patient may decide whether or not to accept this request. If the patient denies the request, then the patient is not added to the caregiver's favorite patient list. If the patient accepts this request, then he/she is added to the caregiver's favorite patient list. When the caregiver provides service to the patient another time, the caregiver can again be queried as to whether or not he/she was satisfied with the patient. If so, then the caregiver can keep the patient on his/her favorite patient list until the caregiver provides service to the patient another time, in which case the query will come up again, and so on. In this manner, the caregiver can be queried about his/her satisfaction with a patient at the end of each service request. If the caregiver is not satisfied with the patient, then he/she may choose to remove the patient from the caregiver's favorite patient list.

A patient can similarly make an addition to his/her favorite caregiver list and/or his/her caregiver blacklist after completion of a service request. The patient may be queried as to whether he/she was satisfied with the caregiver. If the patient was not happy with the caregiver, then he/she may add the caregiver to the patient's caregiver blacklist, in which case the patient and caregiver will not be matched together in future service requests. If the patient is neutral on how he/she feels about the caregiver, then the patient need not take any action, and the caregiver will not be added to the patient's favorite caregiver list or the patient's caregiver blacklist. If the patient is satisfied with the caregiver, then the patient can send the caregiver a request to authorize addition to the patient's favorite caregiver list. In such embodiments, it can be up to the caregiver to decide whether or not to accept this request. If the caregiver denies this request, then the caregiver is not added to the patient's favorite caregiver list. If the caregiver accepts this authorization, then he/she is added to the patient's favorite caregiver list. When the caregiver provides service to the patient another time, the system can query the patient again whether or not he/she was satisfied with the caregiver. If so, then the patient can keep the caregiver on the favorite caregiver list until the caregiver provides service to the patient another time, in which case the query can come up again, and so on. The patient can be queried about his/her satisfaction with a caregiver at the end of each service request. If the patient is not satisfied with the caregiver, then the patient may choose to remove the caregiver from his/her favorite caregiver list. Computing system 100 may be configured to determine which caregiver finished a service request the most number of times, and attempt to add this caregiver to the patient's favorites list. If this caregiver is not available, then computing system 100 may be configured to assign another favorite caregiver who finished a service request for the patient the second most times, the third most times, and so on.

When a patient indicates that he/she wants to add a caregiver or several caregivers to his/her caregiver blacklist, the patient may be prompted to input reason(s). It will be appreciated that skipping over blacklisted caregivers automatically improves the efficiency of service for the patient and the processing speed of computing system 100 in creating dispatch matrix 322 and dispatch output 330. It will also be appreciated that the caregiver blacklists will function as incentive tools for caregivers to improve their overall service, and as incentive tools for patients to behave respectfully if they wish to maintain a particular caregiver. Similarly, the caregiver is incentivized to improve service to gain more business.

A caregiver may refer a patient from his/her favorite patient list to another caregiver. For example, two caregivers involved in such a process may be referred to as Caregiver 1 and Caregiver 2, with the patient referred to as Patient 1. Caregiver 1 may indicate that he wants to refer a favorite patient, Patient 1, to another caregiver, Caregiver 2. An authorization request is first sent to Patient 1 to give him/her the choice to authorize the referral or not. If Patient 1 does not authorize the referral, then no referral occurs. If Patient 1 does authorize the referral, then Caregiver 2 is notified and given the option to accept the referral or not. If Caregiver 2 does not accept the referral, then no referral occurs. If Caregiver 2 accepts the referral, then Caregiver 2 is provided with the service request to complete and Patient 1 may then be added to Caregiver 2's favorite patient list. Similarly, Caregiver 2 may be added to Patient 1's favorite caregiver list.

Users of computing system 100 may make referrals to other users of computing system 100 as long as both users are registered. For users receiving a referral of someone on a blacklist or a favorite list, information such as profile information about the individual being referred can be made available to the user receiving the referral. Computing system 100 can similarly provide information to the referred party about the user to whom they are being referred. For example, a second patient receiving information about a caregiver may see in the profile information of that caregiver that he/she has been found in the past to be a no-show, and the patient may choose not to add that caregiver to his/her favorite list.

Patients may also refer caregivers from their blacklist to other patients. Referring patients can refer individual caregivers, groups of caregivers, or entire lists of blacklisted caregivers to one or several other patients. Caregivers can refer patients from their favorite list to other caregivers. Referring caregivers can refer individual patients, groups of patients or entire favorite lists to one or several other caregivers. A caregiver may include one or more preset reasons or a note describing why he/she is referring the favorite patient or patients. Such a process may start, for example, with Patient 1 indicating that he wants to refer Caregiver 1, who is on Patient 1's favorite caregiver list, to another patient, Patient 2. Before the referral is executed, however, an authorization request is sent to Caregiver 1 who is given the choice to authorize the referral or not. If Caregiver 1 does not authorize the referral, then no referral occurs. If Caregiver 1 accepts the referral, then Patient 2 is given the option to accept the referral or not. If Patient 2 does not accept the referral, then no referral occurs. If Patient 2 accepts the referral, then Caregiver 1 is added to Patient 2's favorite Caregiver list. Similarly, Patient 2 may be added to Caregiver 1's favorite patient list.

Computing system 100 can provide information to a coordinator showing which service requests remain unassigned. Such information can be presented using a map display with labels which identify the service request or the patient number. On this same display, a coordinator might see caregivers near the locations of unassigned service requests. Various indicators may be utilized on the visual display to differentiate an available caregiver from an unavailable caregiver so that the coordinator can know immediately which caregiver(s) to whom he/she should assign to a service request. For example, an available caregiver may be marked by an indicator that shows a white car, whereas an unavailable caregiver may be marked by an indicator that shows a black car. It will be understood by one skilled in the art that the indicators described herein are not meant to be limiting, but rather are illustrative as exemplary embodiments of the present invention.

Accordingly, the system can provide sets of indicators to better assist users, including caregivers and patients, schedule service or get service prescheduled. Various sets of indicators may be provided to streamline the conveyance of information. Indicators can depict this information through one or more letters, numbers, icons, symbols or other graphic representations of the information, which can be displayed on a user's interface. For example, a set of indicators can be provided to convey an estimated travel time (ETT) from the patient's visit location to the patient's drop-off location specified in a service request. A service request may contain multiple pick-ups and drop-offs if the service request is pre-scheduled to have more than one leg. This set of indicators conveying ETT connects location information associated with the patient's visit location and the patient's drop-off location in a prescheduled service request. Preferably, such indicators are displayed to inform the patient about an estimated time in transit, or the caregiver about the estimated travel time to the patient's home, and the length of time requested for any services there (e.g., laundry, cooking, cleaning, etc.) to complete the service request. Such indicators may be based on the estimated time to get from the visit location to a desired drop-off location, which can be provided by a third-party mapping API such as Google® Maps. From the point of view of the caregiver, it may be the time from the current location to the visit location and from the visit location to the drop-off location, an additional step. This too may change if the patient's service request is prescheduled to have multiple duties. Such indicators may also include the time it would take the caregiver to return to the return location from the drop-off location, if an applicable return location limitation was set by the caregiver.

A set of indicators may also be provided to identify more specific availability of caregivers. Such indicators may be shown to both patients and coordinators on an electronic map, where a caregiver may only be shown on the electronic map when active. Such indicators may also include information about the caregiver's availability based on the days and/or times the caregiver has preset. For example, such indicators can show availability and how long the period of availability may last, or whether it is indefinite availability. Such indicators can be removed if the caregiver goes off-duty or is currently carrying out a service request. Alternatively, such indicators may be provided to identify whether a patient's service request has been assigned to a caregiver. Such indicators may be shown to coordinators on an electronic map display, thus providing a visual representation of which services are still waiting to be assigned, and they may be configured to automatically disappear from the coordinator's interface if a caregiver is dispatched for the service request (e.g., once the service request is no longer unassigned).

A set of indicators may also be provided to convey information about the current geolocation of a caregiver and the caregiver's return location, if the caregiver has preset a return location. Such indicators may be provided for viewing by a coordinator on his/her touch screen electronic map display, so that the coordinator is able to assign a service request to the most compatible caregiver. In another embodiment, a set of indicators may be provided to convey information about designated visit locations and drop-off locations, and locations from the first and second leg of a service request, as prescheduled by the patient. Such indicators may show service request related location information to a coordinator or a caregiver on a touch screen electronic map display. The touch screen display enables a patient to make selections regarding a service request, and can be equipped with sensors within the touch screen display that are clickable or otherwise react to touch. The signals that are received by the sensors within the touch screen display are what the processor may receive as input, and those signals may be turned into outputs which lead the system to carry out the selection a patient or a caregiver has made.

A set of indicators may also be provided to show to a patient the caregiver's estimated time of arrival (ETA) at the requested visit location. In order to generate a caregiver's ETA, the system, with the assistance of a third-party API such as Waze®, may identify a caregiver's current real-time traveling speed, such as a road speed along a potential route from the caregiver's current location to the prescheduled visit location. Alternatively, a set of indicators may be provided to show to a caregiver the patient's estimated time of readiness (ETR). Such indicators can reveal, for example, when the patient intends to be ready for pick-up if the patient has scheduled a second leg for a service. For example, a patient at a doctor's appointment can enter into one or more of patient devices 130C1 . . . 130Cn that he/she estimates that he/she will be ready for the return service in thirty (30) minutes. The functionality of an ETR indicator allows for improved efficiency in the provision of prescheduled services. It will be appreciated that such prescheduling and ETR estimates can help caregivers cut back on wasted time. With the assistance of the ETR indicators, a caregiver will be kept aware of the patient's current status, and if the ETR happens to be a long enough amount of time, then the caregiver may be able to complete another service request during that time.

This functionality is especially useful on multiple leg services. The first caregiver who provided a service for the first patient on the first leg may be able to decide, based on the ETR specified by the first patient, whether to wait for that first patient on the second leg or to take a second different service request. If the first patient's ETR shows that he/she might not be ready for a while, then the first caregiver might decide to take the second service request, and anticipate completing it by the time he/she is ready for the second leg. Even if the first caregiver is considered a preferred caregiver for that first patient, if the system detects, based on the ETA, that the first caregiver is not able to arrive for the second leg on time, then the system can notify the first patient. At this point, the first patient will have an option to either agree to wait until the preferred caregiver completes his/her second service request, or to request reassignment of the second leg to another caregiver. If the second leg is provided to another caregiver, that other caregiver is sent an estimate about when to be at the visit location for the second leg which may be conveyed by the ETR indicator.

The system provides multiple sets of indicators to better assist a patient select a best matching caregiver for the service request. Extensive customization may be allowed for both a patient and a caregiver. However, various information including but not limited to pricing, identification of the number of completed service requests, familiarity with a patient requesting service, familiarity with a route to a patient in a service request, etc. may be complicated for a user to navigate when making a deal for a service request. As a patient and caregiver need different information, the indicators will differ depending on the information needed. Caregivers and patients are able to customize their experience, expectations and preferences through the various sets of indicators. Exemplary embodiments of the present invention provide at least twenty-six (26) customizable sets of indicators to streamline this comprehensive information. The system will display unified indicators to avoid confusion, but users may also elect to change certain sets of indicators. For example, users may want to replace a default symbol or icon that represents an indicator with their own symbol, such as an emoticon or abbreviation for his/her own user ID. Furthermore, explanations about what each indicator means may also be provided, and both patients and caregivers have the option of turning these explanations on/off, or temporarily hiding them. If they turn them off, they may still able to turn any or all back on. For example, new users may prefer to have explanations on, while those who have used the services for a while and are familiar with them may not need explanations of the meaning of the various sets of indicators. Patients and caregivers may also change the order in which the indicators are displayed if they would like to prioritize one set of indicators over another.

The indicators are a means to transmit or display information related to services to a patient, a caregiver, or both in a simple, fast, and convenient way. Multiple sets of indicators may be used to assist with matching proper service between the two, as well as to allow for customization by both patients and caregivers. Patients and caregivers may have different indicators on their respective interfaces, but also be able to see one another's indicators. Additionally, a tier system may be utilized for some indicators. The tier system may include a range of numbers with a minimum amount and a maximum amount, and each tier representing a different meaning to different patients and caregivers. Below is a brief description of preferred sets of indicators. A more complete description of these indicators is provided in the '783 application, which is incorporated herein by reference in its entirety.

A first set of indicators may identify a user type for caregivers in three categories, including but not limited to, favorites, preferred, and regular caregivers. A second set may also identify a user type for patients in three categories, including but not limited to favorites, preferred, and regular patients. A third set may identify the availability of caregivers, which may connect the time for which the caregivers will remain available for service requests. A fourth set may identify whether a patient is currently requesting a service request, which may connect the time for which service requests from the patient will remain available. A fifth set may be based on the service request volume that a caregiver has carried out, where numbers regarding historical data are connected. That is, they may connect numbers to reflect the total number of service requests carried out by a caregiver within certain period of time, where the total number is further divided according to a certain period of time and is reflected by numbers, tiers, or combination thereof. A sixth set may show one or more or any combination of geographic zones based on one or more or any combination of corresponding search parameters preset by the patient. A seventh set may be based on the data and information provided by the sixth set of indicators to further compare the total number of potential available caregivers with the total number of potential available patients. An eighth set may help streamline pricing information regarding a caregiver's response to a price proposal from a patient by connecting numbers regarding pricing. A ninth set may display information regarding the negotiation of a price for a caregiver's services based on a price that has been initiated by a caregiver in the case a patient sends the service request without a quoted price, and the caregiver responds with a proposed price, which may be either negotiable or non-negotiable. A tenth set may connect historical data and geolocation data to reflect information regarding zone information based on the total number of service requests completed by a caregiver within the geographic zone in which a patient indicates the visit location the service request. An eleventh set may connect historical data and geolocation data to reflect information regarding zone information based on the total number of requests completed by a caregiver within the geographic zone in which a patient indicated the drop-off location in the service request.

A twelfth set may convey the level of familiarity of a caregiver with the patient providing the service request, represented at least through percentage or any other depiction such as tiers, wherein the familiarity is calculated by the overall level of familiarity a caregiver has with a given patient, based on the patient's history, medical needs, general preferences, etc. An administrator of computing system 100 such as a coordinator may utilize features unique to their job functions coordinating caregivers with various patients. Such coordination may be done automatically through software, manually by human coordinators making decisions about who is likely available to service a service request, or some combination thereof.

This set of indicators may be generated by matching a patient's needs and preferences with a caregiver's service records stored in a database. For a transport request, this information may include the caregiver's familiarity with the route indicated in the service request; for home care service, this information may include the caregiver's level of experience with certain needs of the patient, including the caregiver's past experiences with the patient; and for delivery service, this may be information such as the caregiver's experience or familiarity with the route. Regardless of the service request type, the indicator is adjusted to display information relevant to the situation. The indicator may be displayed by itself as a percentage, or it may be divided into tiers, for example, tier E standing in for familiarity between 0-19 percent, tier D for 20-39 percent, tier C for 40-59 percent, tier B for 60-79 percent and tier A for 80-100 percent. The representation of tier need not be limited to A through E or even letters at all, as tiers may be shown as any single one or combination of depictions that divides the familiarity percentages by tier. The tier and percentage may also be shown together. For a caregiver, this may be a useful indicator in evaluating where he/she has picked up many patients, or where he/she picked up only a few patients. This set of indicators may be used by caregivers to evaluate where they have the most experience or may be the most valuable, or it may be used by a patient in selecting a best matching caregiver based on the experience he/she may have with the service request.

A caregiver's familiarity is contextualized in two ways, by direct or indirect familiarity, and each type is distinct. Direct familiarity is a calculation of how familiar the caregiver is with a certain patient and his/her specific needs, desires and preferences. Indirect familiarity, in contrast, is a calculation of how familiar a caregiver is with the patient in general and the specific requested needs of the patient requesting service but not having specifically worked with the patient in the past (or only a limited number of times). For example, two caregivers may have 100 percent familiarity with a patient, with the first caregiver having provided the same kind of service for a patient previously. This is direct familiarity, and in addition to the 100 percent rating for matching, it may be indicated by number or by tier that this caregiver has provided service to the same patient. The second caregiver also has a 100 percent familiarity—however, part or all of the familiarity for the requested service was provided to a different patient in the past. Even though the caregiver has provided the requested service in its entirety before, it was not provided to the specific requesting patient. The familiarity the caregiver has with the service request is therefore indirect. The degree of familiarity with the service request, whether direct or indirect, will be calculated by comparing the service request with the details of service requests previously completed by the same caregiver by tracking who the services were provided to, and how common all or any parts of the service requests are to the previously completed service requests. The caregiver's familiarity with the service request and the patient making the request would especially matter at times when any part of the service request requires specific knowledge of the given patient's disposition, preferences, needs, etc.

A thirteenth set of indicators may be based on how many times a patient and a caregiver have been matched and have completed a transaction together. A fourteenth set may connect the location information of the caregiver with location information of the patient's visit location to display the time within which a caregiver can pick-up the patient, an estimated time of arrival (ETA). A fifteenth set may be based on the estimated travel time (ETT) from the patient's visit location to the patient's drop-off location indicated in a service request. A sixteenth set may connect geolocation data to reflect at least the patient's pick-up and drop-off locations indicated in the service request. A seventeenth set may convey information about total number of service requests requested and completed by a patient. An eighteenth set may identify one or more or any combination of geographic zones based on one or more or any combination of search parameters preset by the caregiver. A nineteenth set may be based on the eighteenth set of indicators to further display the number of potential available caregivers compared to the number of potential available patients. A twentieth set may streamline the display of the price proposals a patient initiates, where numbers regarding pricing information are connected and displayed. A twenty-first set may provide details about a patient's proposed price that comes as a response to a caregiver proposed price, where numbers regarding pricing information are connected and displayed by this set of indicators. A twenty-second set may connect historical data regarding a patient's service request history and geolocation data to identify a patient by how many service requests he/she has requested and completed based on visit location geographic zones. A twenty-third set may identify a patient by how many service requests he/she has requested and completed based on drop-off location geographic zones, where geolocation data and historical service request data are connected. A twenty-fourth set may connect historical service request data to display the number of times a caregiver and a patient have been matched with each other and completed a transaction together. A twenty-fifth set may identify the estimated travel time to complete a service request for a caregiver. And, a twenty-sixth set may connect geolocation data of a caregiver and the geolocation data of caregiver's preset return location after carrying out a service request, if the caregiver preset a return location.

In addition, any indicator that is based at least in part on countable numbers may also be reflected in terms of tiers. Tiers assigned by the system may be ranges of numbers with minimum and maximum amounts depending on whether it is applied to patients or to caregivers, and also depending on whether it is applied to transport service, delivery service or both transport and delivery service. They may be displayed as letters, shapes or colors or any other way that shows the difference between each tier. Some patients' and caregivers' indicators may have different meanings. The indicators indicate activity relative to their user type, category and subcategory. Since the system stores service request records in the database of the system, it is able to quantify when they were made or carried out by using timestamps and where they were carried out by request details. These records regarding the time or other numbers, however, may be scaled up or down, where a patient may divide the time frame of relevance, such as one or more days, one or more months or one or more years. All of these different adjustable search parameters regarding time or timeframe or zone or other numbers are designed as customizable so that a patient and a caregiver can individualize and prioritize the information they want to see.

The supply and demand relating to a caregiver can be displayed by search parameters preset by the patient based on a time within which the patient expects the pick-up to occur at the visit location, a distance from the visit location, and the desired number of caregivers with whom the patient wants to negotiate a price for the service request. The relevant information about a caregiver may be displayed to the patient via indicators such that the patient may view each potential caregiver and then enable an interactive price negotiation via the displayed indicators. The search parameter based on time may also create a search radius, and may include identifying all caregivers on all possible routes leading to the visit location, identifying real-time traveling speed along each of the possible routes, multiplying the traveling speed of each caregiver along the possible routes by a maximum time preset by the patient to calculate a maximum distance the caregivers can travel within the maximum time preset by the patient, identifying the visit location of the patient as a center point of the search radius, and identifying the extreme point for the search radius, which may be the furthest location of a caregiver within the search parameter or some other point that is at a distance preset by the patient. The supply and demand for the service request within the search parameters may be displayed for the patient as the number of potential available caregivers for supply compared to the number of potential available patients for demand.

Presetting the search parameters by the patient may include presetting and searching for the desired number of caregivers, and creating a search radius by identifying the visit location as the center point for the search radius, and identifying the extreme point for the search radius to be the furthest location from the center point of the caregivers. The search radius may be smaller or larger than a search radius which is based on time or distance, and an estimated time of arrival of the caregivers may be identified for the patient with assistance of the indicators. The number of caregivers identified within the search radius may be prioritized for price negotiation for the patient.

Patients and caregivers may have different indicators displayed on their own interfaces. Some of these indicators may be relevant to the type of user, whether providing or requesting service. Preferably, some indicators are more useful to patients, whereas some are more useful to caregivers. However, indicators are not intended to be displayed to only caregivers or to only patients exclusively, as indicators that are preferably displayed to one party may reflect information useful for the other party. If users choose, however, they can look at their own information through the system. If they are interested in seeing their own indicators or their service request history to make sure that the indicators other users see regarding their service request history are correct, they may do so through their profile information or any other means designated by the system, which shows all the indicators that apply to them. This service request history may include information that other parties can see, such as indicators regarding total number of service requests completed or requested. The service request history may also provide information that other parties cannot see, such as private information regarding how much money they have earned or spent. All such indicators are intended to provide patients and caregivers with customizable information that may help them choose a best matching caregiver or patient, respectively, to make a best deal for each other. It will be appreciated that additional indicators may be developed and/or utilized for deployment through one or more computing devices as a means for visually communicating a summary of other types of relevant information.

The first action a caregiver may take to accept a service request is pressing an ‘Accept’ button on caregiver device 132D1 . . . 132D. When the caregiver is ready to begin the service, he/she can press a ‘Start’ button to indicate that he/she is on the way to the visit location. The caregiver may also call the patient to give notice that he/she is on the way. If at any point after pressing the ‘Start’ button the caregiver needs to make a cancellation, he/she may do so, if, for example, he/she gets a flat tire, or if the caregiver's vehicle experiences a mechanical failure, or any other legitimate grounds for a cancellation. In such circumstances, the caregiver can provide the reasons for the cancellation, and cancel the service request, in which case the service request may be re-dispatched to another caregiver such as a replacement caregiver.

Caregivers may cancel the service requests any time they want. If caregivers cancel one day in advance, then they may not need to report any reason for the cancellation. In such cases, coordinators can reschedule and re-dispatch to a new caregiver. If caregivers cancel on the date that the service requests are to be completed, then caregivers may provide reasoning for the cancellation, choosing from potential default reasons or filling in the reasons by text entry. If no caregivers are readily available to accept a service request following the cancellation, then the service request can go back to the service requests list on a main web portal for coordinators to assign to other caregivers. After a coordinator reschedules and re-dispatches service requests, computing system 100 can be configured to send another notification to the patient, which can include the previous caregiver's name, the reason why the previous caregiver cannot provide service for the patient, the new caregiver's name, the new caregiver's phone number, the new caregiver's vehicle make, brand and color, the visit start time, etc. The patient may change or cancel a service request at any time. If upon entering the vehicle a patient wishes to change destinations, the caregiver may reroute to the intended destination. Once the caregiver and the patient arrive at the drop-off location, the caregiver can ask the patient to sign for the service request as confirmation that correct and/or satisfactory service has occurred. Upon completion of these steps, the service request comes to an end.

It will be appreciated that prescheduling the dispatch of caregivers gives individual caregivers discretion in choosing which service requests to accept or reject. Computing system 100 may provide a functionality for a caregiver to choose potential service requests with, for example, an electronic map showing available routes for that day, the next day, the next week, etc. A caregiver's time and location limitations are taken into account by utilizing current and/or future predicted traffic and other events potentially impacting travel time (e.g., construction, road closures, accidents, etc.) or location limitations on the caregiver. Such data may be stored in database 108 and supplied into dispatch matrix 322 as part of the traffic and map data 326 (see FIG. 3).

Turning next to FIG. 4B, shown is a flowchart illustrating an exemplary series of steps for a patient or caregiver to cancel a prescheduled service request following the exemplary dispatch operation of FIG. 4A, and a new process for replacing the canceling caregiver. A caregiver or a patient may cancel a pre-scheduled service request at any time. If a caregiver (or patient) cancels a prescheduled service request far enough in advance (e.g., one day, one week, etc.), then the caregiver (or patient) need not report the reasoning for the cancellation. In such circumstances, coordinators or computing system 100 simply cancels the service request if cancelled by the patient, or reschedules and re-dispatches the service request to a new caregiver. However, a different process may be utilized for replacing the caregiver who has canceled on short notice.

In particular, as shown in FIG. 4B, once service request 300 has been scheduled to a caregiver (Step 422), computing system 100 can send a notification to the patient (Step 424) indicating that service request 300 has been scheduled. If the caregiver does not cancel (No, Step 426) and the patient does not cancel (No, Step 428), then the service request is carried out (Step 430) as scheduled, and the process ends (Step 432). The caregiver may then begin the billing process. If, however, the patient cancels (Yes, Step 428) through, for example, one of patient devices 130C1 . . . 130Cn, then the patient may be asked to provide the reasons for cancelling (e.g., appointment canceled, driven by another person, service no longer needed, etc.) (Step 434), and the process ends (Step 432) with no billing required. The assigned caregiver may then be dispatched elsewhere. If the caregiver cancels (Yes, Step 426), then computing system 100 must dispatch a new caregiver. In one embodiment computer system 100 dispatches service request 300 based on one or more replacement caregivers identified by the cancelling caregiver if the patient has preset permission to allow replacement caregivers (Step 435). If so (Yes, Step 435), then a notification is sent to the replacement caregiver for acceptance (Step 436). If the patient has not preset permission to allow replacement caregivers, then the service request can be sent to dispatch or the Coordinator for processing (Step 439). The replacement caregiver allocations can be stored in database 108 for quick access by computing system 100. In this case, service request 300 may be sent to the replacement caregiver (Step 436) to complete service request 300. Optionally, service request 300 may be sent to more than one potential replacement caregiver and a list of these best matching replacement caregivers can be displayed for the patient to choose. If the replacement caregiver chosen by the patient accepts the service request (Step 437), then this replacement caregiver services the service request (Step 438).

The method utilized for selecting the replacement caregiver who is to provide service for the service request (or the list of replacement caregivers who might provide service for the service request) can be similar to the methodologies for generating the compatible set of caregivers and dispatch matrix discussed above with respect to FIG. 3. In other words, this may include assigning the potential replacement caregiver a certain caregiver priority, assessing availability, limitations, etc., and the patient may use these considerations to make a selection at his/her discretion. Alternatively, a single replacement caregiver may be associated with each caregiver such that the single replacement caregiver, if available and not on the patient blacklist, is automatically assigned to the service request at the moment the previously assigned caregiver cancels. Upon confirmation of the replacement caregiver as a substitute for the canceling caregiver, the replacement caregiver completes the service (Step 438) and the process ends (Step 432). The replacement caregiver can then initiate the billing process.

If the patient cancels service request 300 far enough in advance (e.g., a threshold of one half hour), then service request 300 can be marked “Cancelled,” and computing system 100 can notify the caregiver immediately. Once the caregiver is sent such cancellation notification, the system may be configured to require that the caregiver confirm receipt of the cancellation. Otherwise, if the caregiver does not confirm receipt of the cancellation, then the application running on the relevant one of caregiver devices 132D1 . . . 132n may be locked such that the caregiver cannot perform any kind of operation within the application until confirming receipt of the cancellation notification. If, however, the patient cancels the service request on short notice (e.g., less than one half hour before the visit start time), then the patient can be assigned a means for identifying that he/she cancelled a service request at the last minute, such as an indication stating “Canceled 5 Min. Before Pick-Up.” After a predetermined number of last minute and/or overall cancellations, the patient may be added to the system's blacklist such that computing system 100 will not preschedule any service requests for that patient for a certain length of time. Computing system 100 may be configured to send a notification to the patient as a warning that after another two cancellations within half an hour, he/she may be put into the system's blacklist. Computing system 100 may also provide contact information in case the patient has any questions.

If a patient's service request is cancelled last minute by a caregiver, then the service request is immediately re-dispatched. The first step of this process is to notify the patient about the cancellation, which may include details of the re-dispatch. In such scenarios, computing system 100 can first determine, for example, the compatibility of the service request with any caregiver on the patient's favorite list. If several are compatible, then the system can notify those caregivers of the available service request, and whichever one of the favorite caregivers accepts the service request first is assigned the service request.

Computing system 100 may be configured to give caregivers the option of adding patients to their preset blacklists. Once a caregiver has added a patient to the caregiver's patient blacklist, the caregiver is skipped over by computing system 100 when searching for matching caregivers for that same patient's service request. In order to avoid confrontation, the system can be configured such that when it receives a service request from a patient who was put on a caregiver's patient blacklist, computing system 100 will automatically send the request to another caregiver and skip any caregivers who added the patient to their respective blacklists.

When a caregiver blacklists a patient, this precludes the same blacklisted patient from being added to the caregiver's favorite patient list. In other words, a blacklisted patient cannot simultaneously be on a caregiver's blacklist and the same caregiver's favorites list. Coordinators may also be notified by computing system 100 when certain caregivers are blacklisted by certain patients, and vice versa, so that they know which caregivers not to dispatch for particular service requests if they want to override computing system 100 for any reason because they have a better or closer caregiver in mind. The blacklist is essentially for difficult patients or caregivers who do not perform well. However, it will be appreciated that a caregiver or a patient can be blacklisted for any reason. Blacklisted patients and caregivers are automatically precluded from being matched with certain corresponding caregivers or patients, or can be precluded from dispatch altogether.

Preset preferences for a patient stored and dynamically updated in database 108 can include, but are not limited to, preferences related to type of caregiver (i.e., favorite, preferred or blacklisted), care experience, physical attributes, ability to perform certain tasks, visit locations, drop-off locations, gender, language spoken, service accessibility, medical device availability, pet accommodation, and baby seat availability. Visit location preference allows a patient to identify his/her visit location. Drop-off location preference allows a patient to identify his/her drop-off location. Years of experience preference allows a patient to preset the number of years' experience he/she may prefer his/her caregiver to have. Gender preference allows a patient to choose a caregiver of a certain gender. Language spoken preference allows a patient to choose a caregiver speaking a certain language. Accessibility preference allows a patient to preset a preference for caregivers whose vehicle is equipped for special accessibility. Medical device availability preference enables a patient to make sure that a transport vehicle has certain equipment, such as oxygen tanks or other medical devices. Pet accommodation preference allows a patient to preset a preference for service requests who are able to accommodate pets. Baby seat availability preference allows a patient to request a caregiver who can provide a baby seat.

A patient might also have a preference for how familiar a caregiver is with the particular route of the service request. A caregiver's familiarity might be direct or indirect familiarity, and both may be conveyed to the patient through a patient interface on the relevant one of patient devices 130C1 . . . 130Cn. Direct familiarity might be, for example, a calculation of how familiar a caregiver is with a route from the visit location to the drop-off location. Indirect familiarity might be a calculation of how familiar a caregiver is with the route between the pick-up and drop-off locations, but through piecemeal experience with the route. For example, two caregivers may have one hundred percent (100%) familiarity with a given route, but through different experiences. The first caregiver may have provided service for the same route, from the same visit location to the same drop-off location. Such experience would constitute direct familiarity, and indicated by, in addition to the 100% rating for matching routes, a number or tier showing that this caregiver has provided service using the same route. The second caregiver may have serviced one part of the route for a different patient in the past, and another part of the route for another service request. Even though this second caregiver has traversed the length of the entire route of the service request, it was not from the same visit location to the same drop-off location. This second caregiver would thus have indirect familiarity with the route of the present service request.

The degree of familiarity with a requested route, whether direct or indirect, can be calculated by comparing the requested route with the routes of service requests previously completed by the same caregiver, and by tracking how common all or any parts of the requested route are to the routes previously completed by the caregiver. It will be appreciated that a caregiver's familiarity with a requested route matters particularly at times when any part of the requested route lies within an area known to have weak or no GPS signal, and/or when the service request takes place after dark, making it harder to follow navigation directions. It will also be appreciated that automated tracking of GPS data on each caregiver and continual storage and updating of such data in the database 108 may be used to verify the caregiver's level of familiarity with a given route.

The systems' customization is in part based on patients' preferences and caregivers' limitations. The concept of preferences discussed herein relates to giving patients options to put certain conditions on service requests. It will be understood by those skilled in the art that the term preference is not intended to restrict that concept in any way, and other terms such as condition, filter condition or qualification may be used. The term limitation is also not intended in any way to restrict the concept of enabling a caregiver to set limits on accepting service requests, and terms such as restriction or boundary may be used. Furthermore, the terms preference and limitation may be used in contrast to one another, and this contrast is intended to convey the difference in user types between patients and caregivers, not to place preferences and limitations as fundamentally opposed concepts. It will be appreciated that a patient who has preferences may also have limitations, or that a caregiver who has limitations may also have preferences.

Service limitations for a caregiver may include, but are not limited to, limitations related service time, return location and time, service area, accessibility, number of passengers, allergy, baby seat limitations, and any other relevant limitations. Service time limitations allow a caregiver to preset the time he/she is not available to provide service. Return location and time limitations allow a caregiver to preset certain locations where he/she wants to be at a certain time. Service area limitations allow a caregiver to preset one or more geographic zones where he/she is not willing to provide services, for example, based on a zip code, borough, city, state, etc. A caregiver may set limitations for geographic zones through an interactive map on a caregiver interface of the relevant one of caregiver devices 132D1 . . . 132Dn. A caregiver may click on a zip code on a map to determine a location where he/she does or does not want to provide service. Such caregiver limitations regarding selection of a geographic location may be required in embodiments where service requests are batched and distributed to caregivers in groups as discussed below.

For example, a caregiver in Brooklyn may see a map of the borough divided into subsections which reflect the zip codes within Brooklyn. The caregiver can click each to indicate the particular service areas. A clickable map interface (i.e. an interactive map interface) allows the caregiver to quickly and efficiently set his/her limitations on his/her own terms (e.g., enabling the caregiver to select the geographic region he/she wants to work in). The clickable map of one caregiver may additionally or alternatively be integrated with a clickable map of another caregiver, such as a replacement caregiver. By combining the service areas of a caregiver and a replacement caregiver, computing system 100 could potentially generate and display for a coordinator the scope of where a caregiver and the caregiver's replacement caregivers could provide service. In addition to personal location limitations, a caregiver may preset personal time limitations relating to his/her work shift. Computing system 100 may group zip codes into borough or county, town, city, or state into mapping data in database 108. Once computing system 100 identifies zip codes or other location limitations, it can skip a caregiver with relevant location limitations when generating dispatch matrix 322 and dispatch output 330 such that no service request is sent to that caregiver which involves areas within that caregiver's limitations. One of ordinary skill in the art will appreciate that the list of caregiver's service limitations described herein is not exclusive, and that a caregiver can have other limitations related to his/her service.

Limitations involving location may greatly affect where a caregiver eventually ends up providing services. It will be appreciated that exemplary embodiments of the invention allow for an efficient dispatch system utilizing different defined regions to schedule service requests. Computing system 100 can identify which service requests will take place where, especially the start and end locations, as they relate to the time they are scheduled, and may group them by region. In current industry practices, dispatching is executed almost randomly. In a large city such as New York, a caregiver may be dispatched at random times to random locations. By grouping service requests by geographic area and tracking caregivers, coordinators can preschedule service requests much more efficiently, and caregivers can complete service requests in less time.

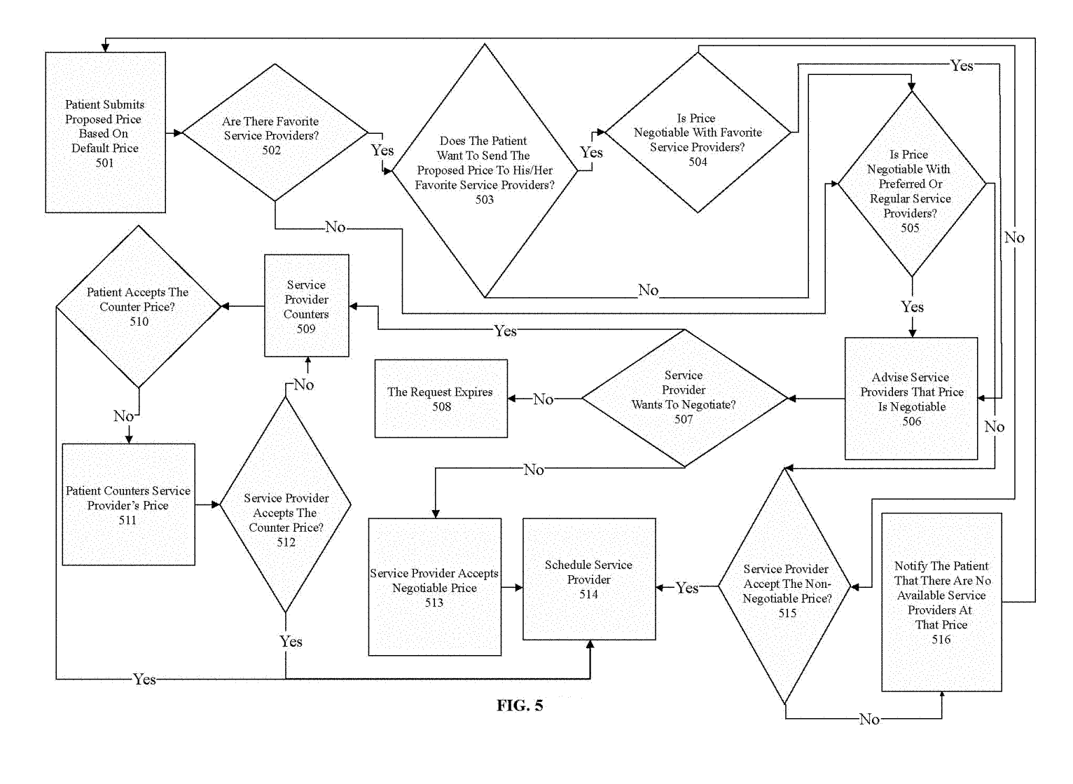

Referring now to FIG. 5, an exemplary workflow of a price negotiation between a service provider (e.g., a caregiver) and a patient is shown. A patient may submit his/her proposed price for a service request to the system (Step 501). The patient's proposed price may be based on a default price, and one or more processors may determine when the proposed price is higher, lower, or equal to the default price. The default price for a transport service may be different from that of a delivery service. When a patient requests a combination of services, the default price may be derived from the default price for each of the transport service and the delivery service. Alternatively, caregivers may upload proposed price offers to provide the services requested by patients, which may be displayed to the patient via indicators. One or more patients and/or caregivers may interactively communicate using the indicators to negotiate an agreed upon price.

For simplicity, FIG. 5 only depicts a scenario in which the patient proposes a price for home healthcare services. The system queries the patient regarding who the patient would like to send the proposed price to, depending on whether the patient has or does not have any favorite caregivers (or any favorite caregivers available) (Decision 502). If there are favorite caregivers available (Yes, Step 502), then the patient can decide whether he/she wants to send the price proposal to one or more of those available favorite caregivers (Decision 503). If the patient wants to send the price proposal to any of those favorite caregivers, he/she additionally decides whether his/her proposed price will be negotiable or non-negotiable with the favorite caregivers (Decision 504). If there are no favorite caregivers, no favorite caregivers available, or if the patient does not want to send out his/her proposed price to any of the favorite caregivers, then the patient can decide whether his/her proposed price will be negotiable or non-negotiable with any preferred or regular caregiver(s) (Decision 505). The process is the same regardless of whether the caregiver is a favorite caregiver or a regular caregiver once it has been established whether the patient will be negotiating or not negotiating with one or more caregivers.

When the patient sets a price he/she deems negotiable, then caregivers will be advised of that (Step 506) via a set of indicators. It is then the caregiver's decision whether to negotiate (Decision 507). The caregiver has three options on a negotiable price proposal. If the caregiver does not want to take the service request at all, he/she declines both negotiation and the request, and the request expires (Step 508). If the caregiver does not want to negotiate but still wants to provide the service request, he/she accepts the original negotiable price as is (Step 513) and is then dispatched (Step 514). The third option available to the caregiver is to negotiate the price. The caregiver makes a counter-offer (Step 509), which is sent to the patient and displayed via one or more sets of indicators. Then the patient may choose whether he/she accepts the counter-offer (Decision 510). If the patient accepts the counter-offer, the caregiver is dispatched by the system (Step 514). If the patient does not accept the counter-offer, then the patient counters the counter-offer (Step 511), and the negotiation cycles back to the caregiver. The caregiver is then presented with a decision of whether to accept the counter-offer price (Decision 512). If the caregiver accepts the counter-offer price, then the caregiver is dispatched (Step 514). If the caregiver does not accept the counter-offer price, then he/she counters the patient's counter-offer (Step 509). The process repeats until the caregiver and the patient both agree on the price, whereupon the caregiver is dispatched (Step 514).

When the patient sets a price he/she deems non-negotiable, the request is routed to the patient's favorite caregivers (or any other caregivers if the patient does not have available caregivers on his/her favorites list), who either accept or don't accept the non-negotiable price (Decision 515). If the caregiver accepts the non-negotiable price, then he/she is dispatched (Step 514). If the caregiver does not accept the request, then the patient is notified that the service request has not been accepted at the price he/she proposed (Step 516). The patient may then, upon notification, revise the original service request and resubmit a new price proposal (Step 501).

One of ordinary skill in the art will appreciate that FIG. 5 only depicts one of many possible scenarios for price negotiation between a caregiver and a patient, and that the system could receive an initial price proposal from either a caregiver or a patient. Accordingly, the initial price proposal could be countered by both a patient and a caregiver. In addition, one of ordinary skill in the art will appreciate that the process depicted herein is not exclusive and does not require the particular order shown to achieve desirable results.

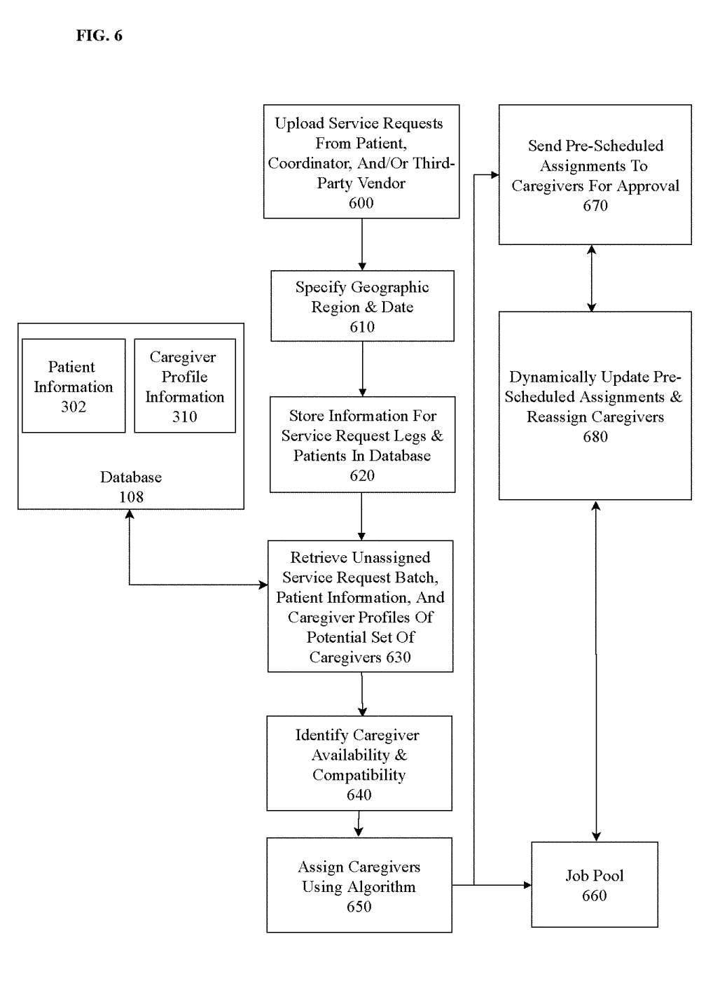

Turning now to FIG. 6, shown is a flowchart illustrating an exemplary methodology for prescheduling a batch (i.e., a plurality) of service requests. A plurality of service requests may be received by computing system 100 (e.g., uploaded by patients and/or by a third-party vendor such as an insurance company on behalf of its patients in need of services) and assigned respective identification numbers (IDs) (Step 600). Such uploads can be made by the patient using one or more of patient computing devices 130C1 . . . 130Cn, electronically by the patient or third-party vendor using vendor device 126 through a web interface, and/or manually by a coordinator using coordinator device 136 after receiving the service request(s) and associated schedules by phone, text message, email, or paper copy. Alternatively, the system may employ interactive voice recognition (IVR) technology and/or voice-to-text technology to automatically receive service requests via traditional telephone systems.

If appropriate, each service request 300 is broken down into duties corresponding to each portion of a service (e.g., visit location(s), drop-off location(s), etc.) (Step 620). A service request may consist of being picked up at location A and dropped off at location B, and at a later time, picked up at location B and dropped off at location A. However, it will be appreciated that a service request may alternatively contain three or more duties and/or different pick-up and drop-off locations, and may spread out over the course of the day. For example, service request 300 might include a patient's home (location A), a hospital (location B), and place of work (location C), and have three duties, A to B, B to C, and C to A. The three duties would take place chronologically, but with different time periods between them. The service request, the duties thereof, and all information associated therewith, including the zip codes corresponding to the pick-up and drop-off locations of each leg, and any required pick-up and/or visit end times, can be stored in database 108. Alternatively, the duties required by the service request may not include transportation services, but rather may include at-home services for patients, which can comprise any number of duties, such as, but not limited to, cooking, cleaning, laundry, helping patients shower and dress, nursing work, counseling, physical therapy, or any other at-home service the patient needs.

For prescheduled service requests, a coordinator can use coordinator device 136 to specify a particular geographic region (e.g., one of the five boroughs of New York City) and a particular date (e.g., the same day, the following day, several days from the present day, a week from the present day, etc.) on which he/she wishes to assign caregivers to service requests in computing system 100 (Step 610). Alternatively, computer system 100 may be preset to automatically batch and assign service requests a predetermined number of days before the requested date of service. For example, computer system 100 may be preset to perform the batched assignment process on August 17^{th} for service requests scheduled for August 18^{th}. The coordinator may view, via an interface on coordinator device 136, a list of the received service requests in computing system 100 chronologically or otherwise by their respective service request dates. The coordinator or computer system 100 might simply begin with the earliest future date for which service requests remain in computing system 100. Computing system 100 may also be configured to automatically select the particular date (e.g., the earliest date for which any service requests remain in the system) and the first geographic region to schedule (e.g., Manhattan, Queens, etc.) on that date.

Following receipt of the particular date and the particular geographic region, computing system 100 retrieves a batch of service requests from the stored plurality of service requests uploaded (Step 630). The retrieved batch of service requests preferably has duties with pick-up and drop-off locations falling within the particular geographic region specified (or with at least one of the respective pick-up and drop-off locations falling within the particular geographic region specified), and have requested pick-up and/or visit end times on the particular date (the designated date) (Step 620). Computing system 100 may also retrieve patient information corresponding to a plurality of patients associated with the batch of service requests, and caregiver profiles corresponding to a potential set of caregivers to assign to the batch based on the particular geographic region and the particular date specified. The patient information may include a patient ID number or name, service preferences, a favorites list, a preferred list, and a blacklist. The caregiver profiles can include caregiver information comprising one or more service limitations, a service history, a favorites list, a blacklist, and historical data.

Computing system 100 may identify the availability and compatibility of the potential set of caregivers for the service requests of the batch (Step 640), and assign caregivers to a plurality of the service requests of the batch (Step 650). It will be appreciated that the system can execute steps 640 and 650 prior to receipt of a dispatch date, whereby caregivers are preliminarily assigned to service requests and dynamically updated using the algorithm (step 650) prior to receiving a specific dispatch date from a coordinator, at which point the system prints a proposed schedule for the designated dispatch date. As shown, computing system 100 cycles between identifying available and compatible caregivers and assigning caregivers since each assignment may likely change the availability of a particular caregiver with respect to the next service request being prescheduled (Steps 640, 650). The availability of the caregivers can be assessed based on the caregiver's preset limitations, and an estimated location of the caregiver at the visit start time of the service request (e.g., the caregiver may already be assigned to an earlier service request leg stored in database 108) whose drop-off location and visit end time will make it feasible/unfeasible for the caregiver to be available to provide service for the service request.

For example, if the final leg of a prior assigned service request has a request for a drop-off at location X₁ at approximately time T₁, and the first leg has a request for pick-up at location X₂ at approximately time T₂, then if locations X₁ and X₂ are relatively close to one another (e.g., within the same zip code) and times T₁ and T₂ are relatively close in time such that a caregiver would have time to get from X₁ at time T₁ to X₂ at time T₂ (e.g., based on a travel time estimate using the stored traffic and map data 326), then this caregiver may be deemed available by computing system 100 with respect to the service request. In this manner, after accounting for patient and caregiver preferences/limitations, computing system 100 can efficiently assign service requests to caregivers.

The compatibility of the potential set of caregivers for the service requests of the batch is assessed in accordance with one or more algorithms or criteria based on, for example, presence of the caregivers on blacklists, favorite lists, or preferred lists of the patients as discussed herein. In preferred embodiments, available caregivers on the patient's favorite list are given the highest priority, followed by available caregivers on a patient's preferred list. Available caregivers on a patient's blacklist are excluded from being assigned to that patient's service request. All available caregivers on a patient's favorites list, and if necessary, preferred list, can be reviewed and considered for potential assignment by computing system 100.

Computing system 100 assigns caregivers according to a predetermined algorithm (Step 650). Computing system 100 can start with a first service request, check caregiver limitations regarding locations, times, conflicts, blacklists, service conflicts, etc., and rule out any caregivers who cannot service the service request due to unavailability, blacklists, expected geographic location at the visit start time, expected lunch breaks or stopping times, or any other reasons. Computing system 100 can next check to determine whether any caregivers not excluded (e.g., who are expected to be available and reasonably close to the visit location at the visit start time and date) are on the patient's favorites or preferred lists. If so, then such caregiver can be accorded high priority.

Optionally, the same caregiver may be assigned to all duties of a service request at Step 650 using compatibility as the leading criteria to maximize client satisfaction. However, it will be appreciated that a second leg (e.g., a return leg) of a service request could potentially be serviced by a different caregiver than the caregiver assigned to the first leg. Alternatively, computing system 100 may assign the first caregiver to the second leg of the first service request while simultaneously keeping the first caregiver's status for the hours in between the two duties as ‘available’, and subsequently assign the first caregiver to another leg of another service request during that available time period should one arise for which the first caregiver is compatible and matched as computing system 100 works through the service requests of the batch. Computing system 100 can be configured to book one or more caregivers to different duties of a service request while also adhering to patient and caregiver preferences and limitations.

It will be appreciated that the overall time during a shift that a caregiver works with a patient minimizes wait times and/or maximizes available work time for a caregiver. It will also be appreciated that dispatch and workflow of caregivers can be updated dynamically as situations arise due to cancellations, delays, or other problems. As service requests are received, grouped, and dispatched ahead of time, the caregivers' work schedules will fill up in advance, and computing system 100 can maximize efficiency, dynamically update the scheduling as caregiver availability changes (e.g., as caregivers call in sick, change their hours, etc.) while catering to patient and caregiver needs.

Computing system 100 may be configured to store any remaining service requests of the batch not assigned to a caregiver in accordance with the algorithms/criteria utilized at Step 650 in a job pool (Step 660) accessible by a plurality of available caregivers through remote caregiver computing devices 132D1 . . . 132Dn to enable the caregivers to select at least one of the remaining service requests. Computing system 100 can cap the number of service requests in the job pool that a single caregiver can select.

As illustrated in FIG. 10, the service requests of the job pool can be displayed via a clickable electronic map display 1000 accessible by the caregivers via caregiver devices 132D1 . . . 132Dn. Electronic map display 1000 may display different geographical regions as noted by the six partitions (i.e., I, II, III, IV, V, and VI) corresponding, for example, to a plurality of local geographic regions where the service request pick-up and drop-off locations are located. The service requests can be displayed as clickable indicators 1010. While ‘stars’ are shown, it will be appreciated that any type of shape, icon, character or image may be used, and that more than one type of indicator may be used to reference different things or features. Caregivers may select (e.g., click on), via the relevant one of caregiver devices 132D1 . . . 132Dn, one of the plurality of partitions I thru VI and/or to select one or more of clickable indicators 1010 to request an assignment area or the corresponding service request. A caregiver can also specify his/her initial limitations or other preset preferences for service using such a map display 1000. For example, a caregiver can designate, as a default setting or for one or more days, a preferred working area in computing system 100 by selecting one of the six partitions I thru VI. A caregiver's default preferred working area can be set at one corresponding to the caregiver's home address.

Referring again to FIG. 6, once a particular caregiver has been assigned to enough service requests and he/she has minimal availability remaining for the designated date, computing system 100 can send an overview of the caregiver's pre-scheduled services for review and approval (Step 670). The caregiver can accept or reject each service request in the schedule. While such notification to and approval by the caregiver can take place, for example, days or even weeks before the designated date when service requests are to be serviced, such notification may be provided close to or even at the beginning of the scheduled date.

A patient may also add a caregiver to his/her favorites list, preferred list, or blacklist at any time, and a caregiver may similarly add a patient to the caregiver's blacklist or favorites list (provided if the request is to add to favorites, the patient agrees to the request). Such additions may occur, for example, following completion of a pre-scheduled service request of the batch. Users may view the profiles of previous caregivers and patients, and to send requests to be on each other's favorites list. A search function can be provided to find the accounts of patients and caregivers. Addition of a patient on a caregiver's blacklist or addition of a caregiver on a patient's blacklist may or may not occur with mutual notification or approval. It will be appreciated that such one-way authorization for blacklists without notification to the other party will help avoid confrontations or awkwardness between patients and caregivers. A caregiver may add a replacement caregiver at any time if both caregivers approve.

When a caregiver rejects (or cancels) a prescheduled service request (Step 670), computing system 100 may be configured to engage a micro-dispatch system and/or process wherein the canceling caregiver dispatches the cancelled pre-scheduled service request to a replacement caregiver. If such replacement caregiver is available and accepts, then computing system 100 can assign the replacement caregiver provided the replacement caregiver is not on the patient's blacklist or vice versa. Such replacement caregivers may also be utilized if, for example, a caregiver accepts a service request but cannot make it to the visit location on time. The caregiver could send the service request leg to his replacement caregiver provided computing system 100 allows this based on replacement caregiver's compatibility with the patient. Each caregiver may have multiple replacement caregivers in the micro-dispatch system and process. The caregiver may be given the option to forward a prescheduled service request that he/she is cancelling to another, non-replacement caregiver, and computing system 100 can assign the cancelled prescheduled request to the other caregiver if the other caregiver accepts, is available, and is deemed compatible with the patient (e.g., not on the patient's blacklist).

If a caregiver rejects any prescheduled assignments (Step 670), then computing system 100 can switch both the caregiver's status during the cancelled time period and the service request's status from assigned to unassigned, identify and assign an alternative caregiver for the service request using the same criteria/algorithm discussed above (Step 680). The prescheduled caregiver assignments stored in the database 108 may be dynamically updated and caregivers reassigned (Step 680). If a caregiver rejects a particular service request on his/her schedule, then computing system 100 may add the rejected service request to the job pool (Step 660), or may automatically reassign the service request to a replacement caregiver, provided such replacement caregiver is available. The job pool may be dynamically updated continually as caregivers approve or reject the schedule of service requests sent to them, in whole or in part. Such updates can be stored in the database 108.

Computing system 100 may also be configured such that if a caregiver rejects any prescheduled service requests or duties thereof, then that caregiver may lose priority. It will be appreciated that based on patient preferences and caregiver limitations, computing system 100 can quickly identify compatible caregivers for an unassigned service request. For example, a caregiver with no limitations and a patient with no preferences can be identified as compatible, and therefore, as a match. Alternatively, if any settings (e.g., caregiver limitations, patient preferences, etc.) conflict with one another, then the caregiver and patient may be deemed incompatible, and be prevented from being matched. It will also be appreciated that comprehensive disclosure of service request details, a caregiver's limitations, and a patient's preferences will improve the experiences of both patients and caregivers.

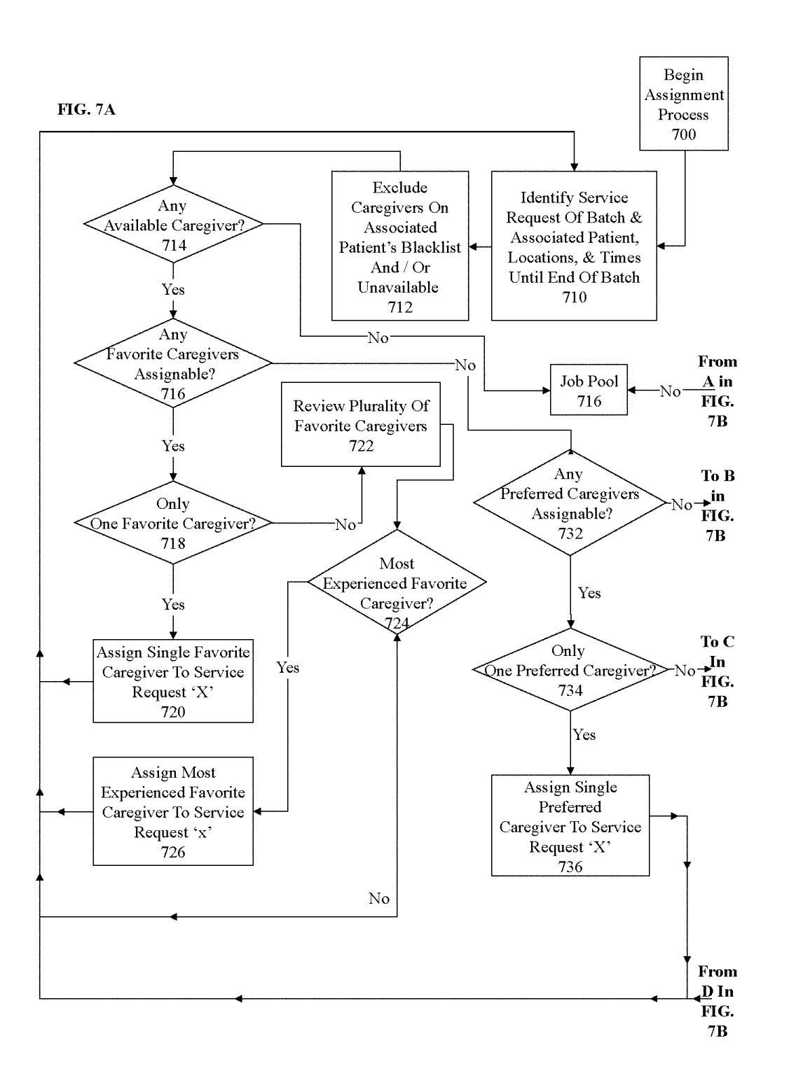

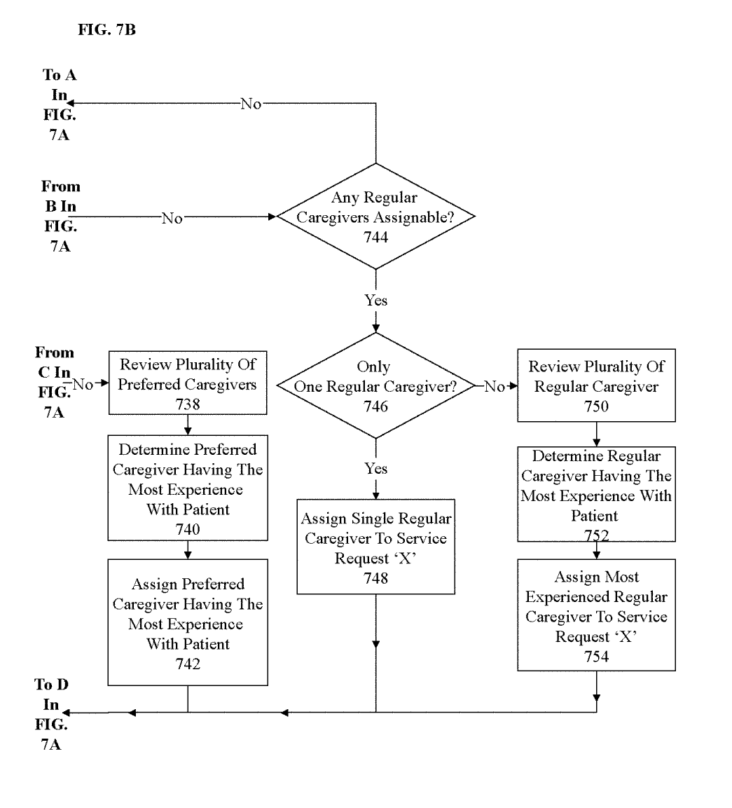

Referring now to FIGS. 7A-7B, shown is a flowchart illustrating a first exemplary algorithm for assigning a plurality (or a batch) of prescheduled service requests in accordance with an exemplary embodiment of the invention. Once a potential set of caregivers and a batch of service requests for a designated date and geographic location have been retrieved by computing system 100 (e.g., at Step 630 of FIG. 6), the process of cycling through the service requests in the batch, and assigning caregivers to as many of them as possible begins (Step 700). Preferably, caregivers in a potential set of caregivers being considered for the batched service requests must have a preset limitation for the geographical region in which he/she will accept jobs.

A first unassigned service request of the batch is identified, as well as the associated patient corresponding thereto, and any pick-up and/or drop-off locations and/or time requirements on the designated date (Step 710). The potential set of caregivers (initially retrieved based on the designated date and the geographic region specified by the coordinator or system) is reviewed and any of the potential set of caregivers who are on the patient's blacklist are excluded, and any who list the patient on his/her blacklist are excluded (Step 712). Whether any of the remaining potential set of caregivers are available to service the service request is then determined (Step 714). Availability can be assessed based on both the caregiver's preset limitations and the estimated location of where the caregiver will be prior to the visit start time of the service request as discussed above. For example, a caregiver who will need to get from location X₁ to location X₂ in time T may be able to do so based on historical traffic and map data 326 stored in database 108, but may have a preset limitation on how far he/she is willing to travel between different duties of respective service requests. If a caregiver is deemed unavailable, then his/her status is set to ‘unavailable’ and he/she is excluded from the potential set of caregivers for this particular service request. If none of the caregivers in the potential set are deemed available, then the service request is sent to a job pool 716 where it can be viewed and potentially selected by other available caregivers as described herein. The assignment process begins again (Step 700) and where it identifies a second unassigned service request of the batch, as well as the new patient, location, and time information associated with the second service request (Step 710).

The system preferably compares the time and location data for each potential caregiver retrieved based on the designated date and the caregiver's preset geographic region with the time and location data from each service request to identify the set of available caregivers. By comparing the time data of each service request and the time preferences or limitations of each potential caregiver, the system can determine which caregivers to preclude from the set of potential caregivers for a given service request. Similarly, the system may compare the pick-up and drop-off location data (and/or the visit start time and visit end time data) to further identify whether a particular caregiver is or will be available, and should be considered for a particular service request. If the service request is for a pick-up or drop-off location (or visit start time and visit end time) outside of a caregiver's preset location and time preferences/limitations, then the system may preclude that caregiver from the set of possible caregivers. Alternatively, the system may be configured to query the particular caregiver who may be identified as a favorite caregiver for a given patient when the service request is for a visit start time/location or visit end time/location that is outside of the caregiver's time/location preferences/limitations in order to give the caregiver the option to accept or reject the service request.

If computing system 100 determines that caregivers are available (Step 714), whether any of the available caregivers are on the particular patient's favorite list is determined (Step 716). Whether only a single favorite caregiver or a plurality of favorite caregivers are available is then determined (Step 718). If only one favorite caregiver is available, then the single favorite caregiver is assigned to the service request (Step 720). The next unassigned service request is then processed (Step 710). If a caregiver, while carrying out a previously assigned service request, receives a second service request assignment, the caregiver may be able to accept the second service request, depending on patient preferences. For example, a caregiver who has preset a return location might be willing to wait for the second service request since the drop-off location and the visit end time of that service request are compatible with the caregiver's return location and time. Preferably, computing system 100 may be configured to avoid empty duties when possible, and the ability to assign a caregiver whose preset return location and preset return time coordinate with the patient's service request is beneficial for the caregiver as he/she can profit from a leg of the service request that might otherwise be empty.

If a plurality of favorite caregivers is available, then computing system 100 can review the plurality of favorite caregivers (Step 722) to determine which of them is the most experienced (e.g., with this patient in terms of the total number of service requests previously performed for this patient, etc.) (Step 724) as indicated by the historical data retrieved at Step 630. It will be appreciated that such historical and other data stored in database 108 can be retrieved at various steps of the process disclosed herein. The most experienced favorite caregiver is assigned to the service request (Step 726). If a conflict arises between two (2) caregivers who have ranked a particular patient the same, then the ranking by the patient may take precedent. That is, if caregiver X and caregiver Y each assign favorite patient A with a ranking of 1 (i.e., a top priority ranking), but patient A ranks favorite caregiver X as a 2 (i.e., a secondary ranking) and favorite caregiver Y as a 1 (i.e., top priority ranking), then favorite caregiver Y may be assigned to favorite patient A. Alternatively, if favorite caregiver X assigns favorite patient A with a ranking of 1 (i.e., a top priority ranking), but favorite caregiver Y assigns favorite patient A with a ranking of 2 (i.e., a secondary ranking), and favorite patient A ranks favorite caregiver X as a 2 (i.e., a secondary ranking) and favorite caregiver Y as a 1 (i.e., top priority ranking), then favorite caregiver X may be assigned to favorite patient A because of favorite caregiver X's higher ranking of the patient. The system may additionally or alternatively be configured to look to the familiarity of each of caregiver X & Y with the patient or the patient's needs, or to the number of historical service requests handled by each of caregiver X & Y to determine the most preferred caregiver for completing the service request.

Alternatively, if a conflict arises between two (2) patients who have ranked a particular caregiver the same, then the ranking by the caregiver of the two patients will take precedent. That is, if each of patient A and patient B assign caregiver X with a ranking of 1 (i.e., a top priority ranking), but caregiver X ranks patient A as a 2 (i.e., a secondary ranking) and patient B as a 1 (i.e., top priority ranking), the caregiver X will be assigned to patient B. Alternatively, if patient A assigns caregiver X with a ranking of 1 (i.e., a top priority ranking), but patient B assigns caregiver X with a ranking of 2 (i.e., a secondary ranking), and caregiver X ranks patient A as a 2 (i.e., a secondary ranking) and patient B as a 1 (i.e., top priority ranking), then caregiver X will be assigned to patient B because of caregiver X's ranking of the patients. Preferably, rankings may comprise a range of numbers (i.e., from 1 to 5, from 1 to 10, etc., where 1 may be the highest priority). Alternatively, the rankings may be identified in the form of top/high, normal/medium, or bottom/low, and the like. Patients and caregivers may rank each of their favorite and preferred caregivers and patients. In preferred embodiments, any favorite caregiver will be considered higher ranked than any preferred caregiver for assignment purposes.

Even when a conflict exists between a caregiver and potential duties of one or more service requests, the system may still assign one or more service requests to a caregiver in the event the system determines the conflict to be a minor one which can be resolved by the caregiver, thus allowing flexibility in the system. The system may determine whether a conflict is minor or severe based on preset values by the caregiver or patient. For example, the caregiver or patient may establish predetermined parameters based on a time or distance from a designated time or designated location of the service request which are deemed to be acceptable. That is, a patient may be willing to accept a 30-minute delay from a favorite caregiver, or may be willing to have a favorite caregiver who is not the closest caregiver but is within, e.g., 5 miles from the patient's location, and still have that caregiver take the job despite a conflict. If, on the other hand, the caregiver is deemed to be outside the predetermined time period or distance, then the system may deem the conflict sufficiently severe to preclude the favorite caregiver and assign the service request to another caregiver.

If a plurality of most experienced favorite caregivers is determined (Step 724), then computing system 100 can determine which of these caregivers will be the closest based on the shortest estimated time period to get from an estimated location on the designated date at the visit start time to the designated visit location of the service request (e.g., as discussed in the example above with respect to locations X₁, X₂ and times T₁ and T₂). The closest experienced favorite caregiver is then assigned to the service request. Once a caregiver has been assigned to the request, (Step 726), the next service request is processed (Step 710).

If none of the remaining potential set of caregivers are on the patient's favorites list, then whether any of the remaining potential set of caregivers is/are on the patient's preferred list is determined (Step 732). If only one preferred caregiver is available (Step 734), then this single preferred caregiver is assigned to the service request (Step 736). If a plurality of preferred caregivers is available (Step 738)(FIG. 7B), then computing system 100 can determine a most compatible or most experienced preferred caregiver and select him/her based on the number of matches between the preset preferences of the patient and the various attributes or limitations of the caregiver (Step 740). This most compatible or most experienced preferred caregiver may be assigned to the service request (Step 742). Historical data may be used to determine which of these preferred caregivers is the most experienced for the patient based on the total number of service requests previously performed for the patient.

Matching attributes between the patient's preset preferences and the caregivers' preset preferences/limitations can include, for example, the following: nationality, accommodation for pets in the patient's home, caregiver's language abilities, caregiver's gender, caregiver's driving experience, caregiver's familiarity with the patient's errand routes, caregiver's experience handling certain types of medical equipment, caregiver's experience handling services such as cooking, cleaning, laundry, nursing, physical therapy, showering, etc., accommodation for other special needs established in the patient's plan of care, or any other attribute or preference. The number of these attributes matched can serve as the basis for assignment of a preferred caregiver to the service request rather than the number of previous services the preferred caregiver performed for the patient. It will also be appreciated that the criteria or algorithm used for selecting one of the plurality of available preferred caregivers may use a combination of these concepts. For example, the preferred caregiver with the most preference matches to the patient may be given priority, then the one with the most previous services for the patient, and then, if two or more preferred caregivers are otherwise deemed equally compatible based on those criteria, the one estimated to be closest based on location and time can be assigned.

When no favorite or preferred caregiver is available, the service request may be assigned to a caregiver categorized as a regular caregiver (Step 744). Computing system 100 first assesses whether only a single caregiver is available or if multiple caregivers are available (Step 746). If only one regular caregiver is available (Step 746), then this single regular caregiver is assigned to the service request (Step 748). If a plurality of preferred caregivers is available (Step 746), then computing system 100 may review the available regular caregivers (Step 750) and determine a most compatible or most experienced regular caregiver (Step 752) and select him/her based on the number of matches between the preset preferences of the patient and the various attributes or limitations of the caregiver. This most compatible or most experienced regular caregiver may be assigned to the service request (Step 754). Historical data may be used to determine which of these regular caregivers is the most experienced for the patient based on the total number of service requests previously performed for the patient or within the particular area. It will be appreciated that one of the caregiver's preset limitations (or the patient's preferences) may preclude assignment of a caregiver to a particular service request at any step. If for any reason, no regular caregivers are available (Step 744), then computing system 100 can put the service request in the job pool (Step 716).

If two or more of the remaining plurality of regular caregivers have previously performed an equal number of service requests for the patient, then computing system 100 can determine a closest caregiver based on the regular caregiver having a shortest estimated time to get from his/her estimated location to the visit location of the service request. Computing system 100 may then assign the closest and most experienced regular caregiver to the service request (Step 754). Once a caregiver has been assigned to the request, (Step 748 or 754), the next service request is processed (Step 710).

In a first exemplary embodiment of the invention, the patient can optionally preset not only his/her favorite caregivers, but also a specific ranking for each favorite caregiver if the patient has multiple favorite caregivers. For example, if a patient has five (5) favorite caregivers on his/her favorites list, then the patient can prioritize these caregivers with preset rankings such as 1^{st}, 2^{nd}, 3^{rd}, 4^{th}, 5^{th}, or 1, 2, 3, 4, 5, or the like, corresponding to each of his/her five favorite caregivers. In certain embodiments, such rankings can vary by day and/or time. It will be appreciated that a number of ranking scenarios are thus possible, as shown, for example, in Table 1 below. As shown, a series of rankings (e.g., from (1) to (5), with (0) meaning no ranking is assigned) are provided by patient Tom for each of five (5) different caregivers (e.g., Ann, Bob, Cathy, David, and Erin).

TABLE 1
Patient's Favorite Caregiver Schedule

| Monday | Tuesday | Wednesday | Thursday | Friday | Saturday | Sunday |
|---|---|---|---|---|---|---|
| Ann (1) | Ann (1) | Ann (1) | Ann (0) | Ann (0) | — | — |
| Bob (2) | Bob (2) | Bob (2) | Bob (0) | Bob (0) | — | — |
| Cathy (3) | Cathy (3) | Cathy (3) | Cathy (0) | Cathy (0) | — | — |
| David (4) | David (4) | David (4) | David (0) | David (0) | — | — |
| Erin (5) | Erin (4) | Erin (4) | Erin (0) | Erin (0) | — | — |

As demonstrated in Table 1 above, Monday shows a full ranking by patient Tom of favorite caregivers for Monday, a partial ranking (i.e., because David and Erin are ranked equally) for Tuesday and Wednesday, and no ranking (or equal ranking) (i.e., because all five (5) caregivers are ranked with a (0)) for Thursday and Friday. The system may use these full, partial and/or equal rankings when prescheduling a plurality of service requests to a plurality of caregivers.

In a second exemplary embodiment of the invention, the patient optionally presets his/her preferred caregivers and a specific ranking for each preferred caregiver if the patient has multiple preferred caregivers. For example, if a patient has five (5) preferred caregivers on his/her preferred list, then the patient can prioritize these caregivers with preset rankings such as 1^{st}, 2^{nd}, 3^{rd}, 4^{th}, 5^{th} or 1, 2, 3, 4, 5, or the like, corresponding to each of his/her five preferred caregivers. It will be appreciated that a number of ranking scenarios are thus possible, as shown, for example, in Table 2 below. As shown in Table 2, a series of rankings (e.g., from (1) to (5), with (0) meaning no ranking is assigned) may be provided by patient Tom for each of five (5) different caregivers (e.g., Frank, George, Harry, Irene, and Julie).

TABLE 2
Patient's Preferred Caregiver Schedule

| Monday | Tuesday | Wednesday | Thursday | Friday | Saturday | Sunday |
|---|---|---|---|---|---|---|
| Frank (1) | Frank (1) | Frank (1) | Frank (0) | Frank (0) | — | — |
| George (2) | George (2) | George (2) | George (0) | George (0) | — | — |
| Harry (3) | Harry (3) | Harry (3) | Harry (0) | Harry (0) | — | — |
| Irene (4) | Irene (4) | Irene (4) | Irene (0) | Irene (0) | — | — |
| Julie (5) | Julie (4) | Julie (4) | Julie (0) | Julie (0) | — | — |

Like Table 1 above, Table 2 shows a full ranking of preferred caregivers on Monday, a partial ranking for Tuesday and Wednesday, and no rankings (or equal rankings) for Thursday and Friday.

In a third exemplary embodiment of the invention, the patient optionally presets his/her favorite and preferred caregivers and a specific ranking for each favorite caregiver and each preferred caregiver if the patient has multiple favorite caregivers and/or multiple preferred caregivers. For example, if a patient has three (3) favorite caregivers on his/her favorites list, and two (2) preferred caregivers on his/her preferred list, then the patient can prioritize these caregivers with preset rankings such as 1^{st}, 2^{nd}, 3^{rd} or 1, 2, 3, or the like, corresponding to each of his/her five favorite or preferred caregivers. A patient can also preset two or more caregivers with the same favorite ranking or preferred ranking. It will be appreciated that a number of ranking scenarios are thus possible, as shown, for example, in Table 3 below. As shown, a series of rankings (e.g., from (1) to (5), with (0) meaning no ranking is assigned) are provided by patient Tom for each of five (5) different favorite caregivers (e.g., Ann, Bob, Cathy, David, and Erin), and for each of five (5) different preferred caregivers (e.g., Frank, George, Harry, Irene, and Julie).

TABLE 3
Patient's Combined Favorite/Preferred Caregiver Schedule

| Monday | Tuesday | Wednesday | Thursday | Friday | Saturday | Sunday |
|---|---|---|---|---|---|---|
| Favorites | Favorites | Favorites | Favorites | Favorites | Favorites | Favorites |
| Ann (1) | Ann (1) | Ann (1) | Ann (0) | Ann (0) | — | — |
| Bob (2) | Bob (2) | Bob (2) | Bob (0) | Bob (0) | — | — |
| Cathy (3) | Cathy (3) | Cathy (3) | Cathy (0) | Cathy (0) | — | — |
| David (4) | David (4) | David (4) | David (0) | David (0) | — | — |
| Erin (5) | Erin (4) | Erin (4) | Erin (0) | Erin (0) | — | — |
| Preferred | Preferred | Preferred | Preferred | Preferred | Preferred | Preferred |
| Frank (1) | Frank (1) | Frank (1) | Frank (0) | Frank (0) | — | — |
| George (2) | George (2) | George (2) | George (0) | George (0) | — | — |
| Harry (3) | Harry (3) | Harry (3) | Harry (0) | Harry (0) | — | — |
| Irene (4) | Irene (4) | Irene (4) | Irene (0) | Irene (0) | — | — |
| Julie (5) | Julie (4) | Julie (4) | Julie (0) | Julie (0) | — | — |

Like Table 1 above, Table 3 shows a full ranking of both favorite and preferred caregivers on Monday, a partial ranking for Tuesday and Wednesday, and no rankings (or equal rankings) for Thursday and Friday.

In a fourth exemplary embodiment of the invention, the caregiver optionally presets his/her favorite patients and a specific ranking for each favorite patient if the caregiver has multiple favorite patients. For example, if a caregiver has five (5) favorite patients on his/her favorites list, then the caregiver can prioritize these patients with preset rankings such as 1^{st}, 2^{nd}, 3^{rd}, 4^{th}, 5^{th} or 1, 2, 3, 4, 5, or the like, corresponding to each of his/her five favorite patients. It will be appreciated that a number of ranking scenarios are thus possible, as shown, for example, in Table 4 below. As shown, a series of rankings (e.g., from (1) to (5), with (0) meaning no ranking assigned) are provided by caregiver Terrance for each of five (5) different patients (e.g., Aaron, Bill, Chris, Drew, and Edward).

TABLE 4
Caregiver's Favorite Patient Schedule

| Monday | Tuesday | Wednesday | Thursday | Friday | Saturday | Sunday |
|---|---|---|---|---|---|---|
| Aaron (1) | Aaron (1) | Aaron (1) | Aaron (0) | Aaron (0) | — | — |
| Bill (2) | Bill (2) | Bill (2) | Bill (0) | Bill (0) | — | — |
| Chris (3) | Chris (3) | Chris (3) | Chris (0) | Chris (0) | — | — |
| Drew (4) | Drew (4) | Drew (4) | Drew (0) | Drew (0) | — | — |
| Edward (5) | Edward (4) | Edward (4) | Edward (0) | Edward (0) | — | — |

As demonstrated in Table 4 above, Monday shows a full ranking by caregiver Terrance of favorite patients is shown for Monday, a partial ranking (i.e., because Drew and Edward are ranked equally) is shown for Tuesday and Wednesday, and no ranking (or equal ranking) (i.e., because all five (5) patients are ranked with a (0)) is shown for Thursday and Friday. The system may use these full, partial and/or equal rankings when prescheduling a plurality of service requests to a plurality of caregivers.

In a fifth exemplary embodiment of the invention, the caregiver optionally presets his/her preferred patients and a specific ranking for each preferred patient if the caregiver has multiple preferred patients. For example, if a caregiver has five (5) preferred patients on his/her preferred list, then the caregiver can prioritize these patients with preset rankings such as 1^{st}, 2^{nd}, 3^{rd}, 4^{th}, 5^{th} or 1, 2, 3, 4, 5, or the like, corresponding to each of his/her five preferred patients. It will be appreciated that a number of ranking scenarios are thus possible, as shown, for example, in Table 5 below. As shown, a series of rankings (e.g., from (1) to (5), with (0) meaning no ranking is assigned) are provided by caregiver Terrance for each of five (5) different patients (e.g., Felice, Gary, Homer, Iris, and Jack).

TABLE 5
Caregiver's Preferred Patient Schedule

| Monday | Tuesday | Wednesday | Thursday | Friday | Saturday | Sunday |
|---|---|---|---|---|---|---|
| Felice (1) | Felice (1) | Felice (1) | Felice (0) | Felice (0) | — | — |
| Gary (2) | Gary (2) | Gary (2) | Gary (0) | Gary (0) | — | — |
| Homer (3) | Homer (3) | Homer (3) | Homer (0) | Homer (0) | — | — |
| Iris (4) | Iris (4) | Iris (4) | Iris (0) | Iris (0) | — | — |
| Jack (5) | Jack (4) | Jack (4) | Jack (0) | Jack (0) | — | — |

Like Table 4 above, Table 5 shows a full ranking of preferred patients on Monday, a partial ranking for Tuesday and Wednesday, and no rankings (or equal rankings) for Thursday and Friday.

In a sixth exemplary embodiment of the invention, the caregiver optionally presets his/her favorite and preferred patients and a specific ranking for each favorite patient and each preferred patient if the caregiver has multiple favorite patients and/or multiple preferred patients. For example, if a caregiver has three (3) favorite patients on his/her favorites list, and two (2) preferred caregivers on his/her preferred list, then the caregiver can prioritize these patients with preset rankings such as 1^{st}, 2^{nd}, 3^{rd}, or 1, 2, 3, or the like, corresponding to each of his/her five favorite or preferred patients. A caregiver can also preset two or more patients with the same favorite ranking or preferred ranking. It will be appreciated that a number of ranking scenarios are thus possible, as shown, for example, in Table 6 below. As shown, a series of rankings (e.g., from (1) to (5), with (0) meaning no ranking is assigned) are provided by caregiver Terrance for each of five (5) different favorite patients (e.g., Aaron, Bill, Chris, Drew, and Edward), and for each of five (5) different preferred patients (e.g., Felice, Gary, Homer, Iris, and Jack).

TABLE 6
Caregiver's Combined Favorite/Preferred Patient Schedule

| Monday | Tuesday | Wednesday | Thursday | Friday | Saturday | Sunday |
|---|---|---|---|---|---|---|
| Favorites | Favorites | Favorites | Favorites | Favorites | Favorites | Favorites |
| Aaron (1) | Aaron (1) | Aaron (1) | Aaron (0) | Aaron (0) | — | — |
| Bill (2) | Bill (2) | Bill (2) | Bill (0) | Bill (0) | — | — |
| Chris (3) | Chris (3) | Chris (3) | Chris (0) | Chris (0) | — | — |
| Drew (4) | Drew (4) | Drew (4) | Drew (0) | Drew (0) | — | — |
| Edward (5) | Edward (4) | Edward (4) | Edward (0) | Edward (0) | — | — |
| Preferred | Preferred | Preferred | Preferred | Preferred | Preferred | Preferred |
| Felice (1) | Felice (1) | Felice (1) | Felice (0) | Felice (0) | — | — |
| Gary (2) | Gary (2) | Gary (2) | Gary (0) | Gary (0) | — | — |
| Homer (3) | Homer (3) | Homer (3) | Homer (0) | Homer (0) | — | — |
| Iris (4) | Iris (4) | Iris (4) | Iris (0) | Iris (0) | — | — |
| Jack (5) | Jack (4) | Jack (4) | Jack (0) | Jack (0) | — | — |

Like Table 4 above, Table 6 shows a full ranking of both favorite and preferred patients on Monday, a partial ranking for Tuesday and Wednesday, and no rankings (or equal rankings) for Thursday and Friday.

As further depicted in the tables above, in a first scenario, classifications (favorite, preferred) may be preset without any rankings among the classified favorite and preferred caregivers/patients (i.e., no rankings or equal rankings). In a second scenario, classifications and rankings of caregivers/patients are both preset (i.e., full rankings). In a third scenario, classifications and rankings of caregivers/patients are both preset, and two or more caregivers/patients within a classification are given the same ranking (i.e., partial rankings). In a fourth scenario, a patient assigns classifications and rankings of caregivers, but one or more caregivers only assign classifications to patients without rankings, etc. If more than one favorite or preferred caregiver of the same ranking is available, then the system can randomly assign the service request to one of these caregivers having the same ranking, or use additional criteria as discussed in various embodiments herein, such as number of prior service requests performed for the patient, level of familiarity with the patient, level of familiarity with the route, etc. Thus, the system may be configured to first look at a patient's available favorite caregivers when assigning a service request, and then assign based on a favorite caregiver rank (i.e., if it identifies multiple favorite caregivers), without regard to which of the favorite caregivers is the most experienced or closest. If there are no available favorite caregivers, then the system can similarly assign based on a preferred caregiver rank. In this manner, a patient can leave little doubt as to the hierarchy of caregiver assignments he/she desires.

In preferred embodiments, the above described calendar variable can be preset by patients or caregivers for each and every preset preference or limitation. For example, when patients input their customizations for favorite caregivers, preferred caregivers, language fluency, type of vehicle, handicap accessible, experience level with cooking, cleaning, etc., such customizable parameters may each be preset in conjunction with the calendar (e.g., the patient may input whether he/she desires the customizable parameter all the time, during a certain time of day, during certain days of the week, and/or during certain months, etc.). Similarly, a caregiver who presets geographic location/region, favorite and preferred patients, days of the week and times he/she desires working or taking a lunch break, etc., each preset may be input in conjunction with the calendar (e.g., the caregiver may input whether he/she desires the customizable parameter all the time, during a certain time of day, during certain days of the week, and/or during certain months, etc.). Patients and/or caregivers can additionally preset holidays or other specific non-recurring days in the year during which (or during certain time periods during which) they do not want to work. In certain embodiments, caregivers can also preset their mode of transportation or preferred mode of transportation (car, bike, public transportation, or some combination thereof). It will also be appreciated that when prescheduling service requests and determining available caregivers, the system will estimate not only the time and distance from the caregiver's current location to the visit location (e.g., home) of the next patient, but also the estimated service time needed at the next patient's home.

As caregivers will generally want as much business as possible, it will be appreciated that the system may be configured to give the patient's preferences priority over the caregiver's preferences. Default settings may alternatively or additionally be utilized if the settings are not completed by a patient or caregiver.

Alternatively, as demonstrated by Tables 1-6 above and Table 7 below, the system may be configured such that the classifications and/or rankings within each classification may be established or customized based on any one or more of the time of day, the day of week, the day of the month, the day of the year, etc. That is, a particular caregiver's or patient's rankings for a given patient or caregiver, respectively, may differ on, for example, different days of the week. As shown in Tables 1-6 above, a given caregiver (e.g., Tom) may rank (i.e., the # in parentheses) each patient (e.g., Ann, Bob, Cathy, David, and Erin) differently depending on the day of the week. As depicted, a ranking of (1) is a top priority, with (2) being a secondary ranking, and so on. For example, as shown in Tables 1-6, caregiver Tom may assign a priority rank for patient Cathy as a (3) on Monday, Tuesday and Friday, but may be rank Cathy as a (1) on Wednesday and Thursday.

In the example(s) shown in Table 7 below, patient Terrance may rank caregiver Chris as a (3) on Monday, Tuesday and Friday, but may rank him as a (1) on Wednesday and Thursday. Similarly, as shown in Table 7, during the morning hours (e.g., from 7:00 am-9:00 am) and evening hours (e.g., from 5:00 pm-7:00 pm) on Mondays, Tuesdays and Fridays, Chris may have a top priority ranking (e.g., a (1)), but over-night (e.g., from 12:00 am-7:00 am and 7:00 pm-12:00 am) and during the daytime hours (e.g., from 9:00 am-5:00 pm) may be assigned a lower ranking (e.g., of (3)). Alternatively, the data shown in Table 7 could be viewed as representing a caregiver's rankings of five (5) separate patients, where the caregiver may rank each patient differently depending on the day of week, the time of day, etc.

TABLE 7
Patient's Favorite Caregiver Schedule (Based on Time of Day)

| | Monday | Tuesday | Wednesday | Thursday | Friday | Saturday | Sunday |
|---|---|---|---|---|---|---|---|
| 12:00am- | Aaron (1) | Aaron (1) | Aaron (1) | Chris (1) | Aaron (1) | — | — |
| 7:00am | Bill (2) | Bill (2) | Bill (1) | Aaron (1) | Bill (2) | | |
| | Chris (3) | Chris (3) | Chris (1) | Bill (2) | Chris (3) | | |
| | Drew (4) | Drew (3) | Drew (1) | Drew (3) | Drew (3) | | |
| | Edward (5) | Edward (3) | Edward (1) | Edward (4) | Edward (4) | | |
| 7:00am- | Chris (1) | Chris (1) | Aaron (1) | Chris (1) | Chris (1) | — | — |
| 9:00am | Aaron (2) | Aaron (1) | Bill (1) | Aaron (1) | Aaron (1) | | |
| | Bill (3) | Bill (2) | Chris (1) | Bill (2) | Bill (2) | | |
| | Drew (4) | Drew (3) | Drew (1) | Drew (3) | Drew (3) | | |
| | Edward (5) | Edward (3) | Edward (1) | Edward (4) | Edward (4) | | |
| 9:00am- | Aaron (1) | Aaron (1) | Aaron (1) | Aaron (1) | Aaron (1) | — | — |
| 5:00pm | Bill (2) | Bill (2) | Bill (1) | Bill (2) | Bill (2) | | |
| | Chris (3) | Chris (3) | Chris (1) | Chris (1) | Chris (3) | | |
| | Drew (4) | Drew (3) | Drew (1) | Drew (3) | Drew (3) | | |
| | Edward (5) | Edward (3) | Edward (1) | Edward (4) | Edward (4) | | |
| 5:00pm- | Chris (1) | Chris (1) | Aaron (1) | Chris (1) | Chris (1) | — | — |
| 7:00pm | Aaron (2) | Aaron (1) | Bill (1) | Aaron (1) | Aaron (1) | | |
| | Bill (3) | Bill (2) | Chris (1) | Bill (2) | Bill (2) | | |
| | Drew (4) | Drew (3) | Drew (1) | Drew (3) | Drew (3) | | |
| | Edward (5) | Edward (3) | Edward (1) | Edward (4) | Edward (4) | | |
| 7:00pm- | Aaron (1) | Aaron (1) | Aaron (1) | Chris (1) | Aaron (1) | — | — |
| 12:00am | Bill (2) | Bill (2) | Bill (1) | Aaron (1) | Bill (2) | | |
| | Chris (3) | Chris (3) | Chris (1) | Bill (2) | Chris (3) | | |
| | Drew (4) | Drew (3) | Drew (1) | Drew (3) | Drew (3) | | |
| | Edward (5) | Edward (3) | Edward (1) | Edward (4) | Edward (4) | | |

Similarly, a patient may rank his/her favorite or preferred caregivers differently by duty/service. For example, favorite caregivers X and Y may be ranked second and third, respectively, for cooking, but fifth and sixth, respectively, for laundry or some other service. In this manner, patients may customize their rankings of not only their favorite and preferred caregivers, but also tie such ratings to particular services of specific caregivers. Thus, if a caregiver provides multiple services, then a patient who uses two or more of the caregiver's services may preset different rankings for that caregiver for each service. As discussed above, such pre-settings can be updated by patients at any time, such as following completion of a service request.

Optionally preset parameters by either the caregivers or the patients may include, for example, a predetermined time, a predetermined distance, or a predetermined acceptable delay. The predetermined time may be established by either or both of the caregiver and/or patient, and represents an amount of time difference the caregiver/patient is willing to allow from the time set in the service request. The predetermined distance may also be established by either or both of the caregiver and/or patient, and represents a distance the caregiver/patient is willing to allow from the location set in the service request. The predetermined acceptable delay may also be established by either or both of the caregiver and/or patient, and may represent an amount of time the patient is delayed from being picked up, dropped off, or from receiving at-home service as set forth in the service request.

Patients can additionally preset whether they want to allow replacement caregivers to be assigned to their service request(s) if specific favorite or preferred caregivers are unavailable or cancel. If a patient does not want any replacement caregivers to handle his/her requests and presets this in the system, then the system can cycle through favorite and preferred caregivers using ranking as priority, and/or using the algorithms discussed herein with respect to FIGS. 7A-9. If a patient does want a replacement caregiver to handle his/her service requests in the event the assigned prescheduled caregiver (e.g., favorite or preferred) cancels or becomes unavailable, then the system can assign a replacement caregiver provided the replacement caregiver is not precluded from being assigned to the patient due to being on the patient's blacklist or failing to meet any other requirement of the patient's service request or preferences. The hierarchy by which replacement caregivers may be woven into assignments for prescheduled service requests (e.g., whether the system should look to the next highest ranking of favorite caregivers or to the replacement caregiver of the favorite caregiver of a specific ranking) may be preset by each patient. Default settings for such algorithms may be utilized if the patient does not preset certain criteria. Thus, the replacement caregiver methodologies disclosed herein may be utilized not only in prescheduling embodiments where a prescheduled assigned caregiver cancels, rejects, or becomes unavailable to service a service request or where a patient changes a service request, but also in the initial prescheduling of a single service request or a plurality of service requests in a given batch.

When more than two or more caregivers replacement with one another as described herein, such grouping creates a micro-dispatching system which mitigates or eliminates traditional manual dispatch. This micro-dispatch system dynamically connects with and updates the general dispatch system 100 through computing devices (e.g., caregiver devices 132 and coordinator device 136) over server 102 and network 124. When service requests are transferred between replacement caregivers, all patient and caregiver preferences and limitations still apply, whereby when service requests cannot transfer between replacement caregivers, the service requests can be routed to the job pool or handled in accordance with any other methodology disclosed herein.

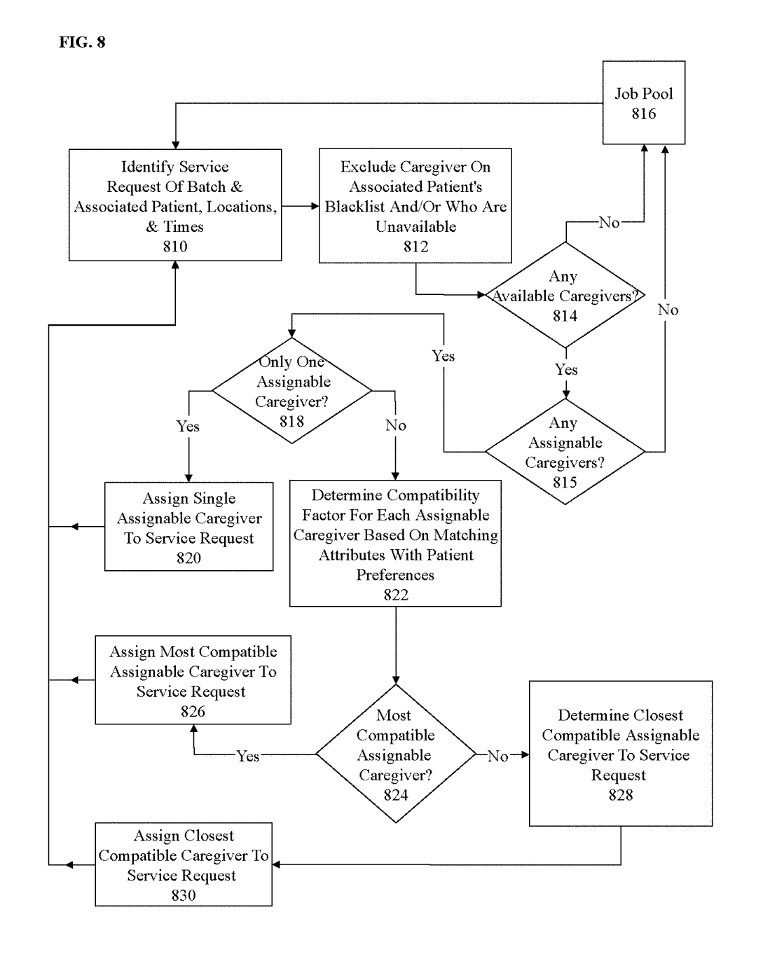

Referring to FIG. 8, a flowchart is shown which depicts a second algorithm for assigning a plurality of prescheduled service requests. As illustrated, an algorithm which prioritizes patient preference and caregiver limitation matching before patient favorite and preferred lists may be used. Once a potential set of caregivers and a batch of service requests for a designated date and geographic location have been retrieved (see FIG. 6, Step 630), computing system 100 is ready to begin cycling through the service requests in the batch to assign caregivers to as many of them as possible. It will be appreciated that favorite and preferred classifications may alternatively be given weighted rankings themselves in addition to other matching criteria utilized (e.g., rather than first looking at classification and rank, and then other matching attributes, the system can be preset to give matching classifications a weighted value in a matching criteria calculation to determine best matching caregivers.

Computing system 100 identifies a first unassigned service request of the batch, including any associated patient-related information, any pick-up/drop-off locations/times, etc. (Step 810). A first potential set of caregivers is reviewed based on the designated date and the geographic region specified by the caregiver's limitations such that certain caregivers may be excluded for being on a patient's blacklist or for having the patient on his/her blacklist (Step 812). Next, whether any of the remaining potential set of caregivers are available (e.g., based on time, location, etc. (see FIGS. 7A-7B)) to service the service request (Step 814). If a caregiver is deemed unavailable, then his/her status may be set to unavailable and he/she will be excluded from the first potential set of caregivers with respect to this particular service request. If none of the caregivers in the first potential set are deemed available, then the service request may be sent to a job pool (Step 816) where it may be viewed and selected by other available caregivers. A second service request of the batch is then identified, as well as the new patient, location, and time information associated with the second service request, and the process continues until all of the service requests of the batch are assigned, and any unassigned requests are sent to the job pool.

If it is determined that potential caregivers are available, then computing system 100 may inquire as to whether such caregivers are also assignable (Step 815) (e.g., whether any patient preferences or caregiver limitations preclude assignment of the service request to such caregivers, whether the caregiver is qualified and/or certified to provide the particular service needed, whether the caregiver has preset whether he/she is open to providing the type of service needed, etc.). If at least one caregiver is found to be assignable, computing system 100 inquires whether there is only one or a plurality of available and assignable caregivers (Step 818). If only one caregiver is available and assignable, then such caregiver is assigned to the service request (Step 820). The next service request is identified for processing.

If more than one available and assignable caregivers are determined, then computing system 100 can determine a compatibility factor for each such caregiver based on matching attributes with patient preferences (Step 822). In certain embodiments, such compatibility factors may take precedence over whether or not any caregivers are on the patient's favorites or preferred lists. Based on patient information and the caregiver profile information, system 100 may preclude one or more caregivers deemed not sufficiently compatible with a patient because of one or more aspects of the service request, patient preferences, and/or caregiver limitations. Such aspects or compatibility factors may be prioritized by the patient in the order in which the patient wants preference to be given. A processing function may be used in which different variables or attributes relating to such preferences, limitations, and/or service request requirements are assigned different priorities and processed. Such matching criteria can be used in accordance with the embodiments discussed with respect to any of the embodiments disclosed in U.S. patent application Ser. No. 15/239,783, entitled “Method and System for On-Demand Customized Services” (“the '783 application”), now published as U.S. Patent Publication No. 20170220966, which is incorporated by reference herein in its entirety.

Computing system 100 determines whether there is a most compatible caregiver (e.g., a caregiver with a highest compatibility factor) (Step 824), and assigns the service request to the most compatible assignable caregiver (Step 826). A new service request is then identified for processing (Step 810). If multiple compatible caregivers are determined to have the highest compatibility factors but are equal or substantially equal, then computing system 100 may estimate which of such caregivers will be the closest to the visit location of the service request based on location and time (Step 828). Computing system 100 then assigns the service request to the closest of the multiple compatible caregivers remaining with the highest compatibility factors (Step 830), and the next service request to process is identified (Step 810).

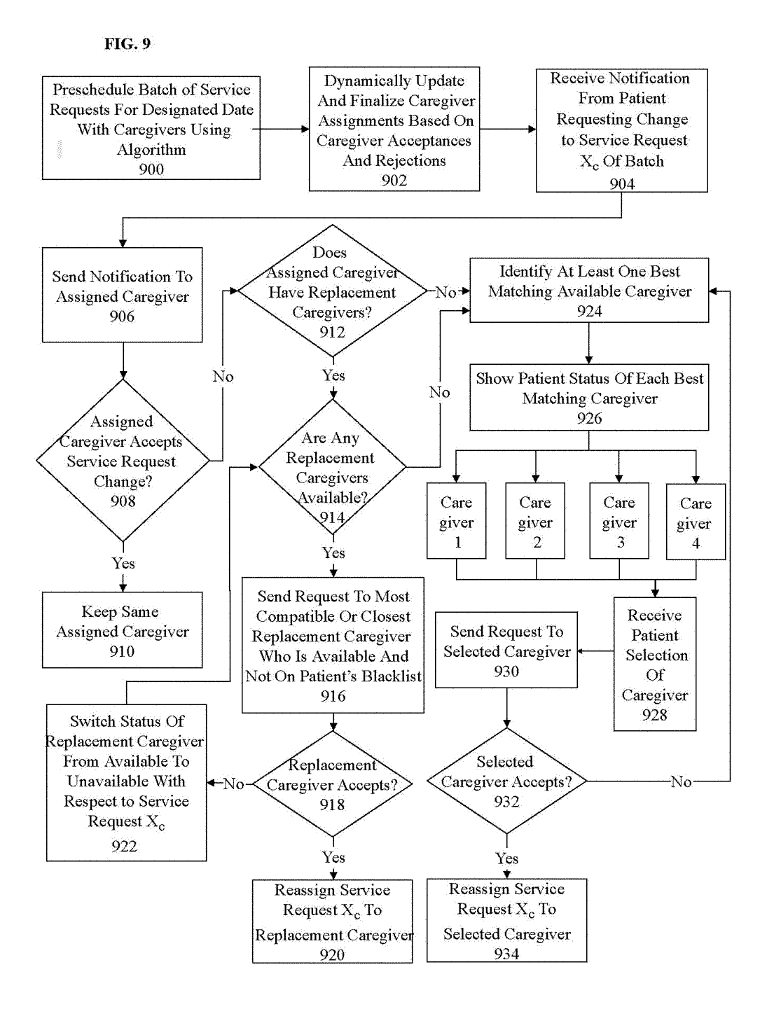

Turning now to FIG. 9, shown is a flowchart illustrating an exemplary methodology for prescheduling and rescheduling a plurality of service requests of a retrieved batch using replacement caregiver and on-demand methodologies in accordance with various embodiments of the present invention. Computing system 100 may preschedule a batch of service requests (Step 900), for example, on Aug. 15, 2017, for a particular designated date of, for example, Aug. 18, 2017, with caregivers utilizing one of the algorithms or criteria discussed herein (e.g., the algorithms shown and discussed regarding FIGS. 7A, 7B and 8). In this example, between Aug. 15, 2017 and Aug. 17, 2017, computing system 100 will preferably dynamically update and finalize caregiver assignments for the batch as caregivers accept/reject the service request assignments given to them, and computing system 100 will reassign the service requests in accordance with various embodiments discussed herein (Step 902). A notification is then received on the designated dispatch date (e.g., Aug. 18, 2017) from a patient corresponding to a particular service request, X_{C}, of the batch indicating that the patient needs to change the visit start time of the service request from 2:00 PM to 5:00 PM (Step 904). It will be appreciated that the patient may alternatively desire other changes, such as, for example, the location of a pick-up or any other attribute of the service request or patient preferences associated therewith.

Upon receipt of the requested change(s) from the patient, computing system 100 notifies the prescheduled caregiver for the service request, X_{C} (Step 906). In certain embodiments, the patient may be enabled to communicate directly with the assigned caregiver to request the change(s). Such communication may take place, for example, directly from the relevant patient device 130C1 . . . 130Cn to the caregiver device 132D1 . . . 132Dn, the vendor device 126, the administrator device 134, the coordinator device 136, or some combination thereof. The assigned caregiver is prompted to indicate whether he/she accepts the change(s) to the service request (Step 908). It will be appreciated that accepting a change to the service request may cause a conflict with another of the assigned caregiver's service requests, and that computing system 100 may be configured to allow the assigned caregiver to accept the change regardless of its effect on later service requests, or may be configured to prevent the caregiver from accepting the change, and to reassign a new caregiver to the service request such as a replacement caregiver. Alternatively, a caregiver may be allowed to accept the change if it is anticipated to only cause some disruption to subsequent service requests. If the assigned caregiver accepts the change (Step 908), then computing system 100 keeps the same assigned caregiver assigned to the service request (Step 910).

If the assigned caregiver does not accept the change (Step 908), then computing system 100 may identify whether the assigned caregiver has any replacement caregivers (Step 912). If so, computing system 100 may identify whether any of the replacement caregivers are or will be available to service the service request X_{C} based on their current or estimated locations, any potential conflicts such as blacklists, etc., or any conflicts between their limitations and the patient's preferences which would preclude an assignment (Step 914). When one or more replacement caregivers are available, then computing system 100 sends a request to the most compatible or closest replacement caregiver who is available (Step 916). Closeness may be established based on location and/or time estimates using both historical and up-to-date traffic and map data 326. Compatibility may be established based on favorites lists, preferred lists, or matching patient preferences and caregiver limitations. The replacement caregiver responds with his/her acceptance or rejection of the request (Step 918). If accepted, computing system 100 reassigns the service request X_{C} to the replacement caregiver (Step 920). If rejected, computing system 100 may automatically switch the status of the replacement caregiver (Step 922) from available to unavailable, and again check for any other available replacement caregivers (Step 914).

When no replacement caregivers are available or when the caregiver does not have any replacement caregivers (No, Steps 912 or No, Step 914), computing system 100 may identify at least one best matching available caregiver (Step 924) using the patient preferences, caregiver limitations, and other matching criteria. As the visit start time draws near for a prescheduled service request, if there are problems (e.g., last-minute caregiver cancellation or patient change, or the service request remains unassigned), computing system 100 can operate in accordance with the various on-demand embodiments described in the '783 application.

The best matching available caregivers can be identified based on matches between patient and caregiver preferences and/or limitations, as well as caregiver availability based on location, time, and preset limitations, if any. Computing system 100 may automatically and periodically track a time-varying geographic location of, for example, the patient devices 103C1 . . . 130Cn and caregiver devices 132D1 . . . 132Dn, and in conjunction with traffic and map data 326 and caregiver location data 324, be able to identify caregivers who may be available to complete the service request. The geographic location of the patient devices 103C1 . . . 130Cn, caregiver devices 132D1 . . . 132Dn, job pools 716/816, electronic map display 1000 may be dynamically updated in database 108, to indicate changes in a patient's and a caregiver's presets, settings, preferences, limitations, feedback or other information to provide quality services. During this on-demand process of identifying at least one best matching available caregiver, a service provider (e.g., caregiver) and a patient can be appropriately matched based on a matrix of patient preferences and caregiver limitations such that patients can be provided last minute assignments with efficiency while also continuing to cater to the patient's personal preferences. By taking into account both the patient's preferences and the caregiver's limitations, more customized experience can be offered that accommodates both parties when scheduling services in advance or on-demand.

Feedback may include positive, neutral, or negative feedback. Positive and negative feedback may be provided in the form of preset positive/negative reasons, respectively, based on evaluation of a provider's performance. The evaluation may be provided either by a patient or a best matching caregiver, who can further choose to add a corresponding party to a favorites list or blacklist. Neutral feedback or no feedback may not affect a match between patients and a caregiver in the assignment of future service requests. Caregivers and patients can mutually be on each other's favorites' lists or blacklists, but cannot be on each other's blacklist and favorites list simultaneously. A patient can add one or more entities to his/her blacklist, and all caregivers affiliated with such entities may also be added to the blacklist.

Alternatively, negative feedback may be provided by preset negative reasons to rate a patient anonymously by putting the patient on a caregiver's blacklist. Negative reasons may include uncleanliness, being a no-show, being too noisy, verbal or physical abuse, refusal of payment or other similar reasons. Similarly, negative feedback may be provided to rate a caregiver anonymously by putting the second caregiver on a patient's blacklist. Here, negative reasons may include rudeness, verbal or physical abuse, messy vehicle, poor driving skills, lack of familiarity with street conditions, inability to follow through with patient's instructions, improper handling of goods, broken or lost goods, late delivery or other similar reasons. Notifications may be provided a certain time after any negative feedback is provided to inform the patient/caregiver that they received negative feedback and why. Based on the negative feedback, a patient and/or caregiver may be suspended from the service.

Once identified, computing system 100 can show the patient a group of best matching caregivers (e.g., Caregiver 1, Caregiver 2, Caregiver 3, Caregiver 4, etc.) with details about each caregiver, expressed through indicators (Step 926). Such details may include distance or time from the patient, route information, route familiarity, etc. Sets of indicators may also be utilized to facilitate a patient in selecting a caregiver from a proposed group of best matching caregivers for his/her service request in a convenient and efficient way, including through negotiating the caregiver's price for the requested services. A patient selects one of the best matching caregivers via one or more inputs to an electronic user interface of patient device 130C (Step 928), and the selected caregiver is then notified with a request to provide service(s) (Step 930). The selected best matching caregiver is queried as to whether he/she would like to accept the service request (Step 932). If the selected caregiver does not accept the service request (No, Step 934), then computing system 100 identifies another best matching available caregiver (Step 924) using the patient preferences, caregiver limitations, and matching criteria in accordance with predetermined rules and algorithms disclosed herein. If the selected caregiver accepts the service request (Yes, Step 932), then computing system 100 reassigns the service request X_{C} to the selected caregiver (Step 934).

It will be appreciated that in certain preferred embodiments, the concepts introduced herein may be utilized in various combinations as desired in order to customize prescheduling services for patients and caregivers for a single prescheduled service request of a single patient, for multiple service requests of a single patient, or for a plurality of service requests for a plurality of patients assigned to one or more caregivers, in advance of or on a designated dispatch date, and that various matching algorithms may be utilized. It will also be appreciated that the prescheduling methods disclosed herein may be utilized in conjunction with on-demand methods, and that the system may switch between prescheduling and on-demand, or vice versa, depending on the situation. For example, a patient may input a single service request into the system for prescheduling and receive a list of best matching caregivers with a set of indicators corresponding to each best matching caregiver based on specific information about each caregiver the patient is interested in viewing (e.g., indicators representative of matching attributes of the caregiver, level of familiarity or experience with a particular patient or route of a service request, etc.), and select one or more of such best matching caregivers to perform the service request. Alternatively, the patient may be notified of a last-minute cancellation by a prescheduled caregiver, and given the option to select from a list of best matching caregivers. Similarly, the system may switch from an on-demand method to a prescheduling method if, for example, a patient who selected a caregiver for on-demand service suddenly learns that his/her doctor is running late, and that he/she now needs to cancel the on-demand service request and instead be picked up in several hours or on another day.

It will be appreciated that the more information patients and caregivers preset, the better the system can customize assignments, relieve or eliminate manual dispatch (e.g., provide automated customizable dispatching), and provide all users of the system increased information and options. The patient may optionally preset one or more aspects of the matching criteria utilized to assign a caregiver to his/her service request(s), or may simply not set any criteria at all other than the terms of the service request itself (e.g., visit start time, visit end time, day, and location).

It will also be appreciated that replacement caregivers may or may not have preferred geographic regions in the same regions as the caregivers from whom they are referred or sent requests, as the replacement caregiver system is meant to minimize dispatching. For example, if a caregiver cancels a service request, then he/she may press a button and see the current status of his/her replacement caregivers and their respective locations, send a recommendation or notification to one of these replacement caregivers. The replacement caregiver can then accept the request if the system allows it (e.g., if the system determines that the replacement caregiver and patient of the service request are not on one or the other's blacklist or precluded because of some other preset criteria or reason). Such functionality can enable caregivers to preserve their business and keep their patients happy if they have to cancel at the last minute.

While available and unavailable have been discussed herein with respect to whether a caregiver is working on a particular date and whether that caregiver will be able to reach a particular designated visit location at a particular designated visit start time based on (1) the expected location of that caregiver on that day and at that time, and (2) the anticipated time for the caregiver to reach the visit location given traffic and map data, it will be appreciated that the system may be configured to be flexible in determining whether a caregiver is available, as a caregiver may be able to make adjustments to service a service request, and may be able to work with a patient to arrange for a slightly different visit start time or location, based at least in part on the dynamically stored data in the database. Such flexibility may be preset in the system by the patient (e.g., a favorite caregiver who can reach the patient a half hour later than the requested visit start time may be acceptable).

In embodiments in which the system preschedules a plurality of service requests on a designated date, each caregiver's preferences as to the geographic region(s) to receive work and the time period(s) to receive work are preferably preset, along with any limitations the caregiver has with respect to geographic locations within the preferred geographic region(s), or limitations with respect to time periods within the caregiver's preferred time period(s). For example, if a caregiver presets specific geographic region(s) in the system (e.g., by clicking on and/or highlighting a specific geographic region of a map in a map interface), then he/she may receive a plurality of prescheduled service requests by the system, each having a visit start time and drop-off location within that geographic region. If there are specific zip codes or locations within that geographic region the caregiver does not wish to service, he/she can specify such limitations within the system, by manual entry or by clicking on a map display interface. If there are specific time periods during which a caregiver prefers to receive work within his/her preset geographic region, then the caregiver can similarly preset such specific time period preferences in the system. If there are specific time periods during which the caregiver does not wish to receive work during that preferred time period (e.g., during his/her lunch break or during a personal appointment during the day), then the caregiver can also preset such time period limitations in the system. In this manner, the system can preschedule a plurality of service requests accurately and efficiently. It will be appreciated that for single prescheduled service requests, patient and caregiver preference with regard to time and location are not required as there is more flexibility in scheduling—caregivers may be individually notified and/or selected by the patient. Thus, a caregiver's preferences for geographic region and time are optionally preset, but are required for being considered to be assigned a plurality of requests on a designated date. When the system contains preset data regarding which geographic regions each caregiver prefers, it can more efficiently assign a plurality of service requests since most service requests will fall within specific geographic regions designated by caregivers (e.g., within Manhattan, Queens, between 30^{th} St. and 90^{th} St., etc.) as opposed to between designated geographic regions.

The system may also recommend or notify various caregivers of service requests sent to or already within the job pool 660, 716 when the system deems such caregivers a good match based on preset data and algorithms the system is configured to execute. For example, the system may recommend a service request in the job pool to a caregiver who suddenly becomes available and/or enters a particular geographic region and/or meets the patient's matching criteria. The system can give the caregiver a predetermined time period to accept. If the caregiver does not accept, then the system may notify another caregiver of the available service request, etc. In other embodiments, the system may notify available caregivers of service requests about to be placed in the job pool (either because an assigned caregiver has cancelled or because the system was not able to preschedule the service request based on preset caregiver preferences), and give such caregivers the ability to select the service request before it is placed in the job pool and/or modify their preset preferences and limitations. Caregivers may be inclined to take such recommended service request as once it is placed in the job pool, a caregiver previously given exclusive rights to it may lose out if another caregiver selects it. The system may also be configured to execute a more inclusive or less restrictive algorithm (e.g., eliminating one or more criteria or reducing the weighting thereof) for service requests in the job pool in order to identify potential caregivers not previously identified using the more restrictive algorithm. The more inclusive algorithm may be less restrictive on, for example, any preset preferences related to availability. For example, caregivers familiar with a geographic region may be able to handle a service request in less time than the system calculates and/or be able to communicate with the patient and agree on a different visit start time. Such communications can help resolve dispatching issues and allow more service requests to be removed from the job pool.

It will be appreciated that new patients to the system, or existing patients who now want service in a new geographic region, may not have favorite or preferred caregivers in the new region(s). Thus, the system may recommend caregivers to the patient and vice versa based on, for example, the caregiver's level of familiarity with a given route and/or the number of service requests the caregiver typically does in the geographic area. All data can be retrieved from the database 108, and analyzed and dynamically updated through the system as patients and caregivers change their preset preferences, as well as following completion of service requests.

Preferably, the database 108 may be dynamically updated with new caregiver location data 324, traffic and map data 326, data from job pool 660, 716, data from map display 1000, and any user interfaces on the patient devices 130_{C1}-130_{CN} and caregiver devices 132_{D1}-132_{Dn} may be dynamically updated in real time when service requests are started, completed, modified, selected, cancelled, assigned, etc.

The best matching caregiver steps discussed herein may additionally or alternatively be utilized for prescheduling a single service request for a patient in combination with, for example, the price negotiation steps discussed with respect to FIG. 5. A patient can review a list of best matching caregivers with respective indicators reflecting a summary of information associated with the particular caregiver, and select one or more for a price negotiation.

It will be appreciated that the sets of indicators disclosed herein can help the patient evaluate supply and demand factors, which may be critical in setting prices for the negotiation as the patient may propose lower prices at times of lower demand or higher prices at times of higher demand. The number of available caregivers who can provide the service and the number of patients currently requesting service in a particular region may be dynamically updated in the database 108. Any caregiver who provides the same type of service as the type of service in the patient's service request can be included in a pool of potential available caregivers as long as the caregiver is within the geographic zone and available. The sets of indicators displayed to the patient may be turned on or off by the patient via the patient device 130. If the patient has turned a set of indicators off, then he/she will not see it on his/her display. However, the patient's service requests may still be counted in a census by the system of all available requests within a proper subcategory.

Various sets of indicators, as described herein, may be displayed to caregivers via caregiver device 132, and may be useful to caregivers for setting a price for the service request based on, for example, various types of patient information. The caregiver may want access to and be provided with the same search results or supply and demand information displayed to the patient. The system may allow the patient and the caregiver to exchange with one another, in full or in part, information displayed to them, which allows for a more transparent negotiation process. It will be appreciated that caregivers may be provided with indicators representative of patient attributes not only for price negotiation, but also for various other embodiments discussed herein. For example, when a caregiver is evaluating whether to select a job from the job pool, or whether to accept a new or available service request he/she is notified about by the system, the system may be configured to enable the caregiver to access patient information associated with the service request, and review indicators associated with the desired patient information. In this manner, the caregiver can make an informed decision as to whether to accept the prescheduled service request.

The system may be configured to utilize a set of indicators to help streamline pricing regarding one or more caregivers' response to a price proposal from a patient, whereby the patient may negotiate price simultaneously with several caregivers. When a patient sends a service request without a quoted price, and the caregiver responds with a proposed price, which may be either negotiable or non-negotiable, the system may display pricing numbers initiated by the caregiver(s) for the patient with a set of indicators. If the proposed prices is/are negotiable, then the negotiation may go back and forth until a final deal or no deal can be made.

Various sets of indicators may be utilized for different situations depending on which party initiates a price proposal. The indicators themselves may convey whether a price proposal is above, below, or equal to a default price provided by the system. For example, the system may display a green indicator or an upward arrow for an initiated price above the default price or a red indicator or a downward arrow for a price that lies below it. Prices that exactly match the default price can be displayed as yellow or as a dot. Those three different colors convey the difference in each of these three situations, and illustrate a clear difference that allows users to sort more quickly, based on an easy-to-see visual representation which would otherwise take longer. Included in the representation is, for example, the actual price proposed, along with information that compares the difference between the price proposal and the default price as a dollar amount or as a percentage. This set of indicators may be turned off, or may selectively have one or any parts displayed depending on the user's discretion. During such a prescheduled service request, the system may be configured to time out the negotiation if no final price is agreed upon, or may give the patient the option of continuing the negotiation up until the date and time of the service request. Users may also set limits on which price proposals are automatically rejected, by presetting a price threshold higher or lower than the default price. Preferably, these sets of indicators are displayed to both a patient and a caregiver. Various sets of indicators may be displayed to any user type in the system. A patient who requests price proposals from a caregiver may preset in the settings not to see any price proposal that is higher than or equal to 50 percent above the default price. Otherwise, the price proposals may be automatically rejected. A patient has discretion in choosing which price indicators to see depending on price difference.

An indicator may also be displayed for a patient reflecting information regarding zone information based on the total number of service requests completed by a caregiver within the geographic zone in which a patient indicates the visit location of the service request. Such indicator may be representative of historical data retrieved from database 108. It will be appreciated that this indicator can provide a patient with a visual representation which quickly conveys the caregiver's experience or familiarity at the visit location. Such information may help inform a patient about which caregiver may be better suited for their service request because of more experience in the area or with a particular type of service.

A set of indicators can display the number of potential available caregivers compared to the number of potential available patients within each geographic zone, the pool of potential available caregivers and the pool of potential available patients, and the geographic zones based on search parameters including number, time and distance or any combination thereof. This set of indicators can provide information about supply and demand within each geographic zone based on the search parameters, which may be factored into setting prices. Caregivers may also limit their displays to show only requests from favorite patients, preferred patients, or from favorite, preferred, regular, and new patients.

It will be appreciated that various embodiments of systems and methods disclosed herein substantially reduce or eliminate manual dispatching by providing automated customized dispatching for home healthcare through computing devices, allow for greater communication between patients, caregivers, and coordinators through dynamically deploying relevant information through the one or more remote computing devices, and enable full service customized automated dispatching, including prescheduling service requests, dispatching service requests, dynamically updating and facilitating changes to service requests, and providing real time information to all parties during transport of patients.

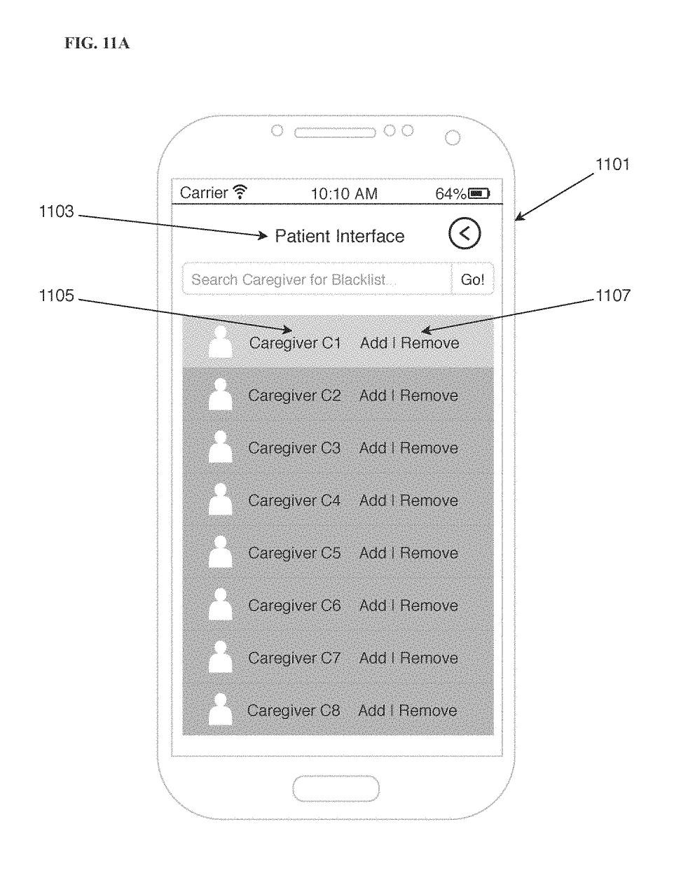

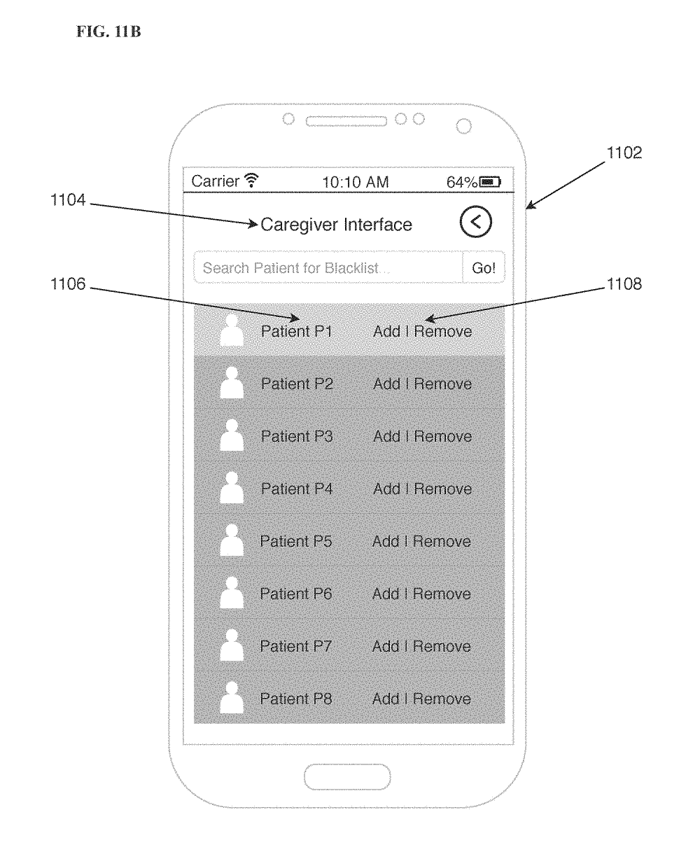

Referring to FIGS. 11A and 11B, exemplary electronic user interfaces 1103, 1104 are shown. Patient electronic user interface 1103 of patient computing device 1101 (e.g., 130C₁) allows a patient to add or remove 1107 one or more caregivers 1105 from the patient's blacklist. Similarly, caregiver exemplary electronic user interface 1104 of caregiver computing device 1102 (e.g., 132D₁) allows a caregiver to add or remove 1108 one or more patients 1106 from the caregiver's blacklist. As shown, the electronic user interfaces may allow patients to search for corresponding caregivers in the system, and vice versa. Such user interfaces may similarly be used to allow patients and caregivers to add and remove one another from their preferred lists or favorite lists. Additionally, the interfaces preferably highlight the current status of a patient or caregiver with respect to whether they are already on one of such lists.

In certain embodiments, the blacklist feature disclosed herein is done anonymously by the patient or the caregiver with respect to all other users of the system. By way of example, if a patient blacklists a caregiver through patient device 130C, then system 100 preferably stores this updated information in database 108, but does not allow the blacklisted caregiver, the coordinator, or any other user of system 100 access to this information. As no other user is made aware of the decision, conflicts or confrontations which would otherwise occur between the user doing the blacklisting and the user being blacklisted may be avoided. Instead, the blacklisted caregiver may notice he or she is no longer being scheduled for service to the patient, but will not know the reason (e.g., the blacklisted caregiver will not know whether the patient moved, switched insurance companies, switched preferred service days or times which are incompatible with the caregiver, chose another caregiver, or simply no longer wants service). Similarly, while a coordinator may notice that the caregiver is no longer listed for providing service, the coordinator will not know the reason either (e.g., the coordinator will not know whether the change was due to updated preferences or limitations input by the patient, the caregiver, or both, or whether the change resulted from one or both parties blacklisting one another. System 100 preferably automatically and continually stores and updates information in database 108 for each caregiver and patient. If a caregiver blacklists a patient, then similarly, system 100 preferably stores the updated information in database 108, but no other users of system 100 are made aware of the blacklist decision or can access this information. The patient will not know, for example, whether the caregiver updated his or her preferred location or times in a manner incompatible with the patient. In this embodiment, only the patient will have access to his or her blacklist and only the patient will know that he/she has blacklisted the caregiver. Use of the word “only” in this context is meant to mean during the normal course of operation of the system and methodology, but it will be appreciated that one or more administrators of the system may also be able to access the patient's blacklist information which “only” the patient can see as this may be necessary in the event of system problems or errors. In preferred embodiments, use of the word “only” here is meant to mean that none of the caregivers (including the blacklisted caregiver) and none of the coordinators can see that a particular patient has blacklisted a particular caregiver. Similarly, if a caregiver blacklists a patient, “only” that caregiver will know that he/she blacklisted the particular patient and will have access to his/her blacklist during the normal course of operation, but one or more system administrators may have access. It will be appreciated that in certain embodiments, system 100 may be configured to allow owners, managers, and/or supervisors to access blacklists for certain purposes such as quality checks or system accuracy. In other embodiments, as described above, the blacklisting is not hidden.

It will appreciated that the above functionality will serve as a face saving gesture with respect to the blacklisted party. A blacklisted caregiver is only effected, for example, with respect to the patient who blacklisted him/her. The decision has no impact with respect to any other patient for whom the caregiver provides service. Additionally, in preferred embodiments, when a patient blacklists a caregiver, this information does not affect any form of overall reputation score or other visible score for the caregiver. Moreover, since the coordinator will not know that the caregiver has been blacklisted, this anonymous functionality will reduce potential bias of the coordinator which might otherwise develop against the caregiver with respect to future assignments.

In certain embodiments, a caregiver may be prescheduled for more than one visit with a patient (e.g., a two month assignment which is three days per week at set times). In such embodiments, if the patient blacklists the caregiver, then system 100 can be configured to provide a notification to the caregiver that he/she is no longer scheduled to provide service to the patient so the caregiver will know not to show up for any more appointments with the patient. Such notification will not give the caregiver the reason for the change/cancellation. Similarly, if a caregiver who is prescheduled for more than one visit with a patient blacklists the patient, then system 100 may be configured to provide a notification to the patient that he/she will be receiving a new caregiver at the scheduled day(s) and time(s) without giving the reason. In certain embodiments, system 100 may additionally transmit a notification to the coordinator indicating that system 100 has automatically rescheduled the change in caregiver (without giving the reason). The notification to the coordinator may display new best matching caregivers on coordinator device 136, and indicate that the caregiver needs to select one. In this manner, the coordinator can verify that the patient receives service in accordance with his/her plan of care.

In certain embodiments, when a first user (e.g., a patient) blacklists a second user (e.g., a caregiver), system 100 may be configured to still allow the blacklisted second user to input a preference that the first user be placed on his/her favorites list or preferred list. The second user will thus not be able to figure out he/she has been blacklisted by the first user. Instead, the second user may merely notice that the first user is not placed on his/her favorites list (and thus infer that the first user has not selected him/her as a favorite), but the second user will not know the reason, and will not know that he/she has been blacklisted by the first user. In other words, in this scenario, the second user will believe that the first user is a preferred user, the first user (and only the first user in preferred embodiments) will know he/she has blacklisted the second user, and the information will be stored in the database but inaccessible by anyone other than the first user. The two users will never be connected, but the blacklisted user will not know he/she has been blacklisted. In yet other embodiments, system 100 may be configured to allow a blacklisted user to become aware of the fact that he or she has been blacklisted by another user, through a query to database 108 and/or a notification from system 100. In certain embodiments, when a caregiver or patient is added to a blacklist, the patient's or caregiver's internal ID is added into a blacklist database table in database 108. In this manner, the blacklist mapping relationships between caregivers and patients are saved. The same may be done for favorites and preferred relationships, and these relationships may be updated dynamically as new inputs are received.

In certain embodiments, if both the first and second users select one another as favorites, then preferably both the first and second users are notified that they have been added to one another's favorite list. “Favorites” requires mutual selection between users, and upon such mutual selection, may result in notifications sent to both parties. However, as described herein, “preferred” lists only require one user selection. By way of example, in a first scenario, if a first user attempts to place a second user on his preferred list and the second user attempts to place the first user on his favorites list, then the first user is placed on second user's preferred list (since the first user requested the second user as a preferred user, not a favorite user) and the second user is placed on the first user's preferred list (since the first user did not reciprocate with a favorite request). In a second scenario, if a first user attempts to place a second user on his preferred list and the second user does not respond, then the first user is not placed on any lists of the second user but the second user is placed on the preferred list of the first user (preferred only requires one direction).

In certain embodiments, a caregiver may work for multiple agencies which subscribe to system 100. System can be configured to identify, by the calendar and scheduling functionalities described herein, whether any conflicts arise or arose (e.g., whether the caregiver indicated he/she was on two different work assignments for two different agencies at the same time). Such functionality can help insurance companies avoid fraud and/or collusion among caregivers.

It will be appreciated by those skilled in the art that all embodiments disclosed herein may be utilized in the transportation industry, where a “driver” instead of a “caregiver” transports a “customer” or “client” instead of a “patient”, and a “dispatcher” instead of a “coordinator” assists with assignments of drivers to customers when needed. In particular, all embodiments disclosed herein may be used in conjunction with the embodiments set forth in U.S. patent application Ser. No. 15/715,012, filed on Sep. 25, 2017 and titled ‘System and Method For Customizable Prescheduled Dispatching For Transportation Services’, and U.S. patent application Ser. No. 15/239,783, filed on Aug. 17, 2016 and titled ‘Method and System for On-Demand Customized Services’, the disclosures of which are incorporated by reference herein in their entireties. Additionally, all embodiments disclosed herein may be used in conjunction with the embodiments set forth in U.S. patent application Ser. No. 15/934,677, filed Mar. 23, 2018 and titled System and Method For Healthcare Billing Verification, the disclosure of which is hereby incorporated by reference herein in its entirety.

It will be understood that the phrases or terminology employed herein are for purposes of description and not of limitation. While the present invention has been shown and described with reference to various preferred embodiments, those skilled in the art will readily appreciate that various changes and/or modifications may be made without departing from the spirit and scope of the invention as defined by the claims. Any exemplary embodiments described herein are merely illustrative, and many variations can be introduced without departing from the spirit of the disclosure or from the scope of the appended claims. For example, elements and/or features of different exemplary embodiments may be combined with each other and/or substituted for each other. The scope of the invention, therefore, shall be defined solely by the following claims, and it will be apparent to those of skill in the art that numerous changes may be made in such details without departing from the spirit and the principles of the invention.

What is claimed is:

1. A computer-implemented method for providing customizable automated prescheduling of home health care services, the method comprising:
receiving, by a server, a plurality of preset caregiver service zones, wherein each preset caregiver service zone is defined by one or more geographic regions;
receiving, by the server, a plurality of preset caregiver preferences or limitations of a plurality of caregivers;
receiving, by the server, a plurality of optionally preset patient preferences of a plurality of patients;
storing, in a database, the plurality of preset caregiver service zones, the plurality of preset caregiver preferences or limitations, and the plurality of optionally preset patient preferences;
receiving, by the server, a plurality of service requests for prescheduling, wherein each service request corresponds to one of the plurality of patients and contains time-location data associated with at least one service location;
parsing, by the server, the plurality of service requests into one or more batches, wherein each batch corresponds to a respective preset caregiver service zone and includes one or more of the plurality of service requests having service locations all within the respective preset caregiver service zone;
prescheduling the plurality of service requests by, for each of the one or more batches:
(i) retrieving, from the database, a set of the plurality of caregivers associated with the respective preset caregiver service zone corresponding to the batch;
(ii) automatically establishing, by the server, in accordance with the one or more predetermined rules, for each respective service request of the batch, a weighted priority for each caregiver of the set, wherein the weighted priority is based on assigned weights for at least one service relevant factor associated with the respective service request, and wherein the at least one service relevant factor includes at least one of the plurality of preset caregiver preferences or limitations or at least one of the optionally preset patient preferences of the patient corresponding to the service request; and
(iii) applying, by the server, the one or more predetermined rules to assign a caregiver from the set to the respective service request based on the weighted priority of the caregiver;
transmitting, by the server to a computing device of a particular caregiver, a dispatch notification which includes one or more assigned service requests; and
receiving, by the server from the computing device of the particular caregiver, an acceptance of at least one of the assigned service requests.

2. The method according to claim 1, wherein the optionally preset patient preferences comprise preferences related to one or more of the following: wheelchair familiarity, laundry, showering, dressing, cooking, cleaning, repair work, one or more health conditions, one or more diseases, paralysis, accommodation for pets, caregiver's language abilities, caregiver's gender, caregiver's physical abilities, caregiver's experience level, or experience handling certain types of conditions.

3. The method according to claim 1, wherein the optionally preset patient preferences include at least one of: the patient prioritizing at least one favorite caregiver on a favorites list of the patient, or the patient prioritizing at least one preferred caregiver on a preferred list of the patient, and
wherein the preset caregiver preferences include the caregiver prioritizing at least one favorite patient on a favorites list of the caregiver.

4. The method according to claim 1, further comprising:
transmitting a redispatch request, by an assigned caregiver, for a respective service request;
sending one or more redispatch notifications to a replacement caregiver associated with the assigned caregiver; and
receiving acceptance of the respective service request from the replacement caregiver.

5. The method according to claim 1, further comprising:
determining a level of familiarity of a respective caregiver with a respective patient based on historical service relevant data stored in the database, wherein the assigned caregiver for each service request is based at least in part on the level of familiarity of the caregiver.

6. The method according to claim 1, wherein the one or more geographic regions comprise at least one of: a country, a state, a city, a borough, a zip code, a neighborhood, a group of streets, a street, or any other region defining feature.

7. The method according to claim 1, further comprising:
enabling a respective caregiver to set at least one of a plurality of geographic service regions or a service time limitation using an interactive electronic interface of the caregiver computing device.

8. A computer-implemented method for providing customizable automated prescheduling for home health care services, the method comprising:
receiving, from a patient device associated with a patient:
a first input requesting that a particular caregiver be placed on a blacklist of the patient, and
a second input corresponding to at least one patient preset preference comprising: a favorite caregiver, a preferred caregiver, or a customizable caregiver attribute;
adding the particular caregiver to the blacklist of the patient;
receiving, from a plurality of caregiver computing devices associated with a plurality of caregivers: a plurality of caregiver preset preferences corresponding to the plurality of caregivers, wherein the plurality of caregiver preset preferences comprise at least one of: a favorite patient, a preferred patient, or a geographic region where a respective caregiver desires to provide services to patients;
receiving at least one preset calendar preference including one or more designated time periods during which the plurality of caregiver preset preferences are applicable, or one or more designated time periods during which the at least one patient preset preference is applicable;
storing the first input, the second input, the plurality of caregiver preset preferences, the blacklist, and the at least one preset calendar preference in a database;
receiving a service request associated with the patient for scheduling, the service request including at least one of: number of hours, authorization level, certification level, type of care, service start time, service location, or service end time; and
automatically scheduling a caregiver to service the service request based on the at least one patient preset preference, the at least one caregiver preset preference, and the at least one calendar preference,
wherein the particular caregiver is precluded from being scheduled to the service request based on the first input, and wherein the blacklist of the patient is accessible by the patient.

9. The method according to claim 8, further comprising:
determining whether any of the plurality of caregivers are on a favorites list of the patient; and
responsive to no caregivers being on the favorites list of the patient, identifying one or more compatible preferred caregivers for the patient based on one or more matching attributes, wherein the one or more matching attributes are determined based one or more of the optionally preset caregiver preferences matching with one or more of the optionally preset patient preferences.

10. The method according to claim 9, further comprising:
responsive to no caregivers being on the favorites list of the patient, assigning the service request to a compatible preferred caregiver having a highest number of matching attributes, or a most experienced preferred caregiver for the patient based on a total number of service requests previously performed for the patient.

11. The method according to claim 9, further comprising:
responsive to no available caregiver of plurality of caregivers being at least one of: a favorite caregiver or a preferred caregiver of the patient, determining a compatible regular caregiver for the patient based on one or more matching attributes between the optionally preset caregiver preferences and the optionally preset patient preferences; and
assigning the service request to the compatible regular caregiver.

12. The method according to claim 8, further comprising:
assigning at least one priority ranking to at least one of a patient classification or at least one of a caregiver classification, wherein the priority ranking is used to assign the service request to the caregiver.

13. A computer-implemented method for providing customizable automated prescheduling for home health care services, the method comprising:
receiving, from a caregiver device associated with a particular caregiver, a first input requesting that a particular patient be placed on a blacklist of the particular caregiver;
receiving, from a patient device of the particular patient, a second input corresponding to at least one patient preset preference comprising: a favorite caregiver, a preferred caregiver, or a customizable caregiver attribute;
adding the particular patient to the blacklist of the particular caregiver;
receiving, from a plurality of caregiver computing devices associated with a plurality of caregivers: a plurality of caregiver preset preferences corresponding to the plurality of caregivers, wherein the plurality of caregiver preset preferences comprise at least one of: a favorite patient, a preferred patient, or a geographic region where a respective caregiver desires to provide services to patients;
receiving at least one preset calendar preference including one or more designated time periods during which the plurality of caregiver preset preferences are applicable, or one or more designated time periods during which the at least one patient preset preference is applicable;
storing the first input, the second input, the plurality of caregiver preset preferences, the blacklist, and the at least one preset calendar preference in a database;
receiving a service request associated with the particular patient for scheduling, the service request including at least one of: number of hours, authorization level, certification level, type of care, service start time, service location, or service end time; and
automatically scheduling a caregiver to service the service request based on the at least one patient preset preference, the at least one caregiver preset preference, and the at least one calendar preference,
wherein the particular caregiver is precluded from being scheduled to the service request based on the first input, and wherein blacklisting of the particular patient by the particular caregiver is accessible by the particular caregiver.

14. The method according to claim 13, further comprising:
determining a dispatch matrix showing compatibility between the particular patient and one or more caregivers based on factors comprising at least one of: availability of each caregiver to accept the service request, the caregiver being on the favorites list of the particular patient, the caregiver being on the preferred list of the particular patient, the caregiver being on the blacklist of the particular patient, or the optionally preset caregiver preferences matching the optionally preset patient preferences.

15. The method according to claim 13, further comprising:
prioritizing one or more caregivers in accordance with one or more of the following: being on a favorites list of the particular patient and available to accept the particular patient's service request, having a highest number of completed service requests for the particular patient, having a lowest initiated price proposal, having a lowest price response to a price proposal initiated by the particular patient, being most familiar with a route between one or more service locations of the service request, having a largest number of service requests carried out within a geographic region of the one or more service locations, having a largest number of service requests carried out within a geographic region of the one or more service locations, having a largest number of service requests completed with the particular patient, having a shortest estimated time of arrival, having a current location closest to the one or more service locations, or having a preset return location compatible with a time and a location of the service request,
wherein the prioritizing is implemented based on the optionally preset patient preferences.

16. The method according to claim 13, the method further comprising:
displaying, on a caregiver computing device, an electronic map display divided into a plurality of clickable geographic regions;
enabling the plurality of caregivers to each select the geographic service region from the electronic map through a respective caregiver computing device; and
updating the database dynamically upon selection of one of the geographic service regions by the plurality of caregivers.

17. The method according to claim 13, further comprising:
determining, when at least one service request is not assigned to at least one caregiver;
storing the service request which is not assigned in a job pool, wherein the job pool is accessible by at least one or more available caregivers through the one or more computing devices; and
enabling the one or more available caregivers to select the service request in the job pool through the one or more computing devices.

18. The method according to claim 17, further comprising:
displaying each unassigned service request in the job pool as one or more clickable icons on an electronic map display; and
updating dynamically the job pool and the electronic map display upon selection of one of the service requests in the job pool by one of the one or more available caregivers, wherein the electronic map is divided into a plurality of geographic regions.

19. The method according to claim 13, wherein the optionally preset caregiver preferences comprise one or more personal limitations or one or more legal limitations;
wherein the one or more personal limitations comprise one or more location limitations based on the geographic service region and one or more other limitations related to one or more of the following: estimated time of arrival to a preset return location, estimated travel time, estimated travel distance, type of services needed, number of services needed, weight of patient, size of patient, one or more allergies, and accommodation for pets,
wherein the one or more legal limitations relate to one or more of the following: local, state, or federal regulations concerning legality or illegality of the one or more caregivers providing services requested for a particular service request in the geographic service region, one or more other limitations related to service time, number of patients, certification level, or authorization level, and
wherein the one or more legal limitations are implemented to preclude a respective caregiver from receiving the service request if the one or more legal limitations prevent the respective caregiver from carrying out the service request.

20. The method according to claim 13, further comprising:
determining, when more than one favorite caregiver is identified in the particular patient's preferences, a top priority favorite caregiver, and, responsive to the top priority favorite caregiver being available, assigning the top priority favorite caregiver to the service request; or
determining, responsive to the top priority favorite caregiver being unavailable, a second priority favorite caregiver, and, responsive to the second priority favorite caregiver being available, assigning the second priority favorite caregiver to the service request.
